(19) United States
(12) Patent Application Publication
(10) Pub. No.: US 2025/0281526 A1
(43) Pub. Date: Sep. 11, 2025

(54) Compositions Comprising Stable Free Available Chlorine Species and Peroxides, Methods of Making and Uses Thereof

(71) Applicant: Collidion, Inc., Petaluma, CA (US)

(72) Inventors: Hojabr Alimi, Petaluma, CA (US); Mohammad Esmaeili, Mashhad (IR)

(73) Assignee: Collidion, Inc., Petaluma, CA (US)

(21) Appl. No.: 19/199,977

(22) Filed: May 6, 2025

Related U.S. Application Data
(63) Continuation-in-part of application No. 18/042,970, filed on Feb. 24, 2023.
(63) Continuation of application No. PCT/US2023/079291, filed on Nov. 9, 2023.
(60) Provisional application No. 63/069,808, filed on Aug. 25, 2020.
(60) Provisional application No. 63/383,062, filed on Nov. 9, 2022.

(51) Int. Cl. A61K 33/20; A61K 33/40; A61P 11/00

(52) U.S. Cl. CPC A61K 33/20; A61K 33/40; A61P 11/00

(57) ABSTRACT

The present specification discloses a method of making a composition stably comprising one or more peroxides and one or more free available chlorine species, one or more peroxide for use in the manufacture of a composition stably comprising one or more peroxides and one or more free available chlorine species, as well as formulated compositions using a composition manufactured according to a method or use disclosed herein.

This is a continuation-in-part application which 1) claims the benefit of priority and is entitled to the filing date of U.S. patent application Ser. No. 18/042,970, filed on Feb. 24, 2023, a 35 U.S.C. § 371 U.S. national stage patent application which claims the benefit of priority and is entitled to the filing date of International Patent Application PCT/US2021/047627, filed Aug. 25, 2021, an international patent application which claims the benefit of priority and is entitled to the filing date pursuant to 35 U.S.C. § 119 (e) of U.S. Provisional Patent Application 63/069,808, filed Aug. 25, 2020, and 2) claims the benefit of priority and is entitled to the filing date of International Patent Application PCT/US2023/079291, filed Nov. 9, 2023, an international patent application which claims the benefit of priority and is entitled to the filing date pursuant to 35 U.S.C. § 119 (e) of U.S. Provisional Patent Application 63/383,062, filed Nov. 9, 2022, the content of each of which is hereby incorporated by reference in its entirety.

Hydrogen peroxide and hypochlorous acid may be used separately for the sterilization of various surfaces, including surgical tools, and may be deployed as a vapor (VHP) for room sterilization and may be used to treat topical infections as well as those infections developed within body cavities. Both compounds demonstrate broad-spectrum efficacy against viruses, bacteria, yeasts and fungi, and bacterial spores. However, since hydrogen peroxide is a strong oxidizer, free available chlorine, which may include hypochlorous acid amongst other forms of chlorine, remains highly unstable in the presence of hydrogen peroxide. Attempts to mix hydrogen peroxide and hypochlorous acid to date have failed to demonstrate a stable aqueous solution containing both compounds. Certain methods of production of hypochlorous acid, such as electrolysis, have cited the potential presence of theoretical peroxide in solution in insignificant and unstable amounts. The present specification disclosed herein pertains to a novel production of hydrogen peroxide which may include other reactive oxygen species in substantial amounts and the benefits of such discovery for producing a solution also comprising HOCl and/or one or more stable chlorine reactive species. As such, making a composition comprising both stable hydrogen peroxide, or other reactive oxygen species, and free available chlorine in the same solution has not been previously documented.

The present specification discloses compositions using added salts to improve the stability and efficacy off a composition comprising a stable free available chlorine alone or a stable free available chlorine that can coexist in the presence of hydrogen peroxide, molecular oxygen, or another reactive oxygen species (ROS). As shown herein, a composition comprising hydrogen peroxide and a stable free available chlorine exhibits significantly improved the cleaning, disinfecting, sanitizing, and/or sterilizing properties, physical properties, and product stability.

### SUMMARY

Aspects of the present specification disclose a composition comprising added metal salts to compositions comprising free available chlorine, or a composition comprising hydrogen peroxide and a stable free available chlorine. Metal salts disclosed herein may include compositions of one of more of sodium chloride, potassium chloride, calcium oxide, calcium chloride, and sodium phosphate.

Other aspects of the present specification disclose a composition comprising hydrogen peroxide and a stable free available chlorine. A composition disclosed herein can further comprise and one or more metallic particles, one or more metal salts and/or a phosphate buffer like calcium phosphate or sodium phosphate. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

Other aspects of the present specification disclose a composition comprising hydrogen peroxide, a stable free available chlorine, and one or more metallic particles. A composition disclosed herein can further comprise one or more metal salts and/or a phosphate buffer like calcium phosphate or sodium phosphate. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

Other aspects of the present specification disclose a composition comprising hydrogen peroxide, a stable free available chlorine, and one or more metal salts. A composition disclosed herein can further comprise one or more metallic particles and/or a phosphate buffer like calcium phosphate or sodium phosphate. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

Other aspects of the present specification disclose a method of making a composition comprising one or more peroxides and a stable free available chlorine. A disclosed method uses a peroxide as a reagent in the manufacture of a stable free available chlorine. One or more peroxides disclosed herein include hydrogen peroxide, sodium peroxide, calcium peroxide, lithium peroxide, potassium peroxide, other alkali metal peroxides, other alkali earth metal peroxides, or a combination thereof. Presence free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. Additionally, the disclosed method produces a composition further comprising one or more reactive oxygen species, including, without limitation, oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃), another reactive oxygen species (ROS), or a combination thereof.

Other aspects of the present specification disclose a method of making a composition comprising hydrogen peroxide and a stable free available chlorine. A disclosed method uses a peroxide disclosed herein as a reagent in the manufacture of a stable free available chlorine. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

Other aspects of the present specification disclose a kit comprising one or more containers including a composition disclosed herein and/or one or more containers including components of a composition disclosed herein. A disclosed kit further comprises one or more delivery or application systems, and/or instructions, and/or a container.

Other aspects of the present specification disclose a method to clean, disinfect, sanitize, and/or sterilize a device. The disclosed method comprises applying a composition disclosed herein to a device, wherein application of the composition cleans, disinfects, sanitizes, and/or sterilizes the device. The disclosed method may further comprise rinsing a cleaned, disinfected, sanitized, and/or sterilized device with a rinse solution disclosed herein. In other aspects of the present specification disclose a composition disclosed herein for use in cleaning, disinfecting, sanitizing, and/or sterilizing a device. In other aspects of the present specification disclose a use of a disclosed composition clean, disinfect, sanitize, and/or sterilize a device.

Other aspects of the present specification disclose a method to clean, disinfect, sanitize, and/or sterilize a surface area. The disclosed method comprises applying a composition disclosed herein to a surface area, wherein application of the composition cleans, disinfects, sanitizes, and/or sterilizes the surface area. The disclosed method may further comprise rinsing a cleaned, disinfected, sanitized, and/or sterilized surface area with a rinse solution disclosed herein. In other aspects of the present specification disclose a composition disclosed herein for use in cleaning, disinfecting, sanitizing, and/or sterilizing a surface area. In other aspects of the present specification disclose a use of a disclosed composition clean, disinfect, sanitize, and/or sterilize a surface area.

Other aspects of the present specification disclose a method to clean, disinfect, sanitize, treat, and/or sterilize a wound in an individual. The disclosed method comprises applying a composition disclosed herein to an individual, wherein application of the composition cleans, disinfects, sanitizes, treat, and/or sterilizes a wound. In other aspects of the present specification disclose a composition disclosed herein for use in cleaning, disinfecting, sanitizing, and/or sterilizing of a wound in an individual. In other aspects of the present specification disclose a use of a disclosed composition clean, disinfect, sanitize, treat, and/or sterilize of a wound in an individual. In other aspects of the present specification disclose a use of a disclosed composition in the manufacture of a medicament to clean, disinfect, sanitize, treat, and/or sterilize of a wound in an individual.

Other aspects of the present specification disclose a method to clean, disinfect, sanitize, treat, and/or sterilize a body tissue or body cavity in an individual. The disclosed method comprises applying a composition disclosed herein to an individual, wherein application of the composition cleans, disinfects, sanitizes, treats, and/or sterilizes a body tissue or body cavity. In other aspects of the present specification disclose a composition disclosed herein for use in cleaning, disinfecting, sanitizing, treating, and/or sterilizing of a body tissue or body cavity in an individual. In other aspects of the present specification disclose a use of a disclosed composition clean, disinfect, sanitize, treat, and/or sterilize of a body tissue or body cavity in an individual. In other aspects of the present specification disclose a use of a disclosed composition in the manufacture of a medicament to clean, disinfect, sanitize, treat, and/or sterilize of a body tissue or body cavity in an individual. A body cavity can be a space, compartment, or lumen, or potential space, compartment, or lumen in the body. A body cavity may include any space, compartment or lumen that contains organs, limbs, tissues, and surfaces within the body, or any space, compartment or lumen within organs, limbs, or tissues. A body cavity may include areas with the organs, for example an airway within the lungs, a nasal cavity, an oral cavity, a lumen of the gastrointestinal or excretory system including urethra and ureter, and spaces within the reproductive organs. A body cavity may be exposed to the outside of the body or may not be exposed to the outside space of the body. A body cavity any space created in the body. A body cavity may also include a space created in the body from injury or surgery or other artificial means.

Other aspects of the present specification disclose a method to clean, disinfect, sanitize, treat, and/or prevent an infection in an individual. The disclosed method comprises applying a composition disclosed herein to an individual, wherein application of the composition cleans, disinfects, sanitizes, treats, and/or prevent an infection. In other aspects of the present specification disclose a composition disclosed herein for use in cleaning, disinfecting, sanitizing, treat, and/or prevent of an infection in an individual. In other aspects of the present specification disclose a use of a disclosed composition clean, disinfect, sanitize, treat, and/or prevent an infection in an individual. In other aspects of the present specification disclose a use of a disclosed composition in the manufacture of a medicament to clean, disinfect, sanitize, treat, and/or prevent an infection in an individual. An infection can be a microbial infection.

Other aspects of the present specification disclose a method to treat or prevent a respiratory infection in an individual. The disclosed method comprises applying a composition disclosed herein to an individual, wherein application of the composition cleans, disinfects, sanitizes, treats, and/or prevents a respiratory infection. In other aspects of the present specification disclose a composition disclosed herein for use in cleaning, disinfecting, sanitizing, treating, and/or preventing a respiratory infection in an individual. In other aspects of the present specification disclose a use of a disclosed composition to clean, disinfect, sanitize, treat, and/or prevent a respiratory infection in an individual. In other aspects of the present specification disclose a use of a disclosed composition in the manufacture of a medicament to clean, disinfect, sanitize, treat, and/or prevent a respiratory infection in an individual. An infection can be a viral, bacterial, or fungal infection. A viral infection can be an infection from a coronoavirus (including SARS-COV-2, MERS, SARS), influenza, Respiratory syncytial virus (RSV), enterovirus, and other viruses.

Other aspects of the present specification disclose a method to treat a wound in an individual. The disclosed method comprising applying a composition disclosed herein to an individual, wherein application of the composition promotes healing of a wound. In other aspects of the present specification disclose a composition disclosed herein for use in treating a wound in an individual. In other aspects of the present specification disclose a use of a disclosed composition to treat a wound in an individual. In other aspects of the present specification disclose a use of a disclosed composition in the manufacture of a medicament to treat a wound in an individual.

Other aspects of the present specification disclose a method to clean, disinfect, sanitize, treat, prevent and/or provide relief an inflammatory response in the respiratory system, including the lung, of an individual. In aspects a disclosed method comprising applying a composition disclosed herein to an individual, wherein application of the composition promotes healing of a respiratory inflammation. In other aspects of the present specification disclose a composition disclosed herein for use in cleaning, disinfecting, sanitizing, treating, preventing and/or providing relief of a respiratory inflammation in an individual. In yet other aspects of the present specification disclose a use of a disclosed composition to treat and/or provide relief from a respiratory inflammation in an individual. In still other aspects of the present specification disclose a use of a disclosed composition in the manufacture of a medicament to clean, disinfect, sanitize, treat, prevent and/or provide relief from a respiratory inflammation in an individual.

### DETAILED DESCRIPTION

Aspects of the present specification disclose a method of combining stable free available chlorine with one or more peroxides in an aqueous solution. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. One or more peroxides disclosed herein include hydrogen peroxide, sodium peroxide, calcium peroxide, lithium peroxide, potassium peroxide, other alkali metal peroxides, other alkali earth metal peroxides, or a combination thereof. Additionally, the disclosed method produces a composition further comprising one or more reactive oxygen species, including, without limitation, oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃), another reactive oxygen species (ROS), or a combination thereof.

Standard understanding of chlorine chemistry has shown combinations of HOCl or FAC with hydrogen peroxide are unstable formulations. An example of such reaction is as follows:

Equation. HOCl (aq)+H₂O₂ (qa)→O₂ (g)+HCl (aq)+H₂O (I)

In this example, the HOCl and peroxide interact to generate oxygen gas (O₂) and an acidic solution. This redox reaction moves rapidly in a unidirectional manner to eliminate the peroxide and HOCl in an aqueous solution to produce water, hydrochloric acid, and oxygen gas.

The disclosed methods involve a novel chemistry that establishes a solution in equilibrium with the addition of sodium peroxide (NA₂O₂) and chlorine gas (Cl₂) in an aqueous solution. Without wishing to be limited to any theory, the proposed chemical equations are believed to be as follows:

$$N {A}_{2} {O}_{2} + 2 {H}_{2} O \to 2 NAOH + {H}_{2} {O}_{2} . \qquad Equation 1$$

$${Cl}_{2} + {H}_{2} O ⇌ HOCl + HCl . \qquad Equation 2$$

$$NaOH + HCl \to {H}_{2} O + NaCl . \qquad Equation 3$$

The importance of the new chemistry involves the improvement of the stability and efficacy of chemistry of hypochlorous acid and other free available chlorines. Salts, when added in the amounts to achieve neutral biological osmolality, improved the stability of free chlorine and pH of the composition. Over time, free chlorine composition decreases in amount of free chlorine and pH. The addition of salts to the composition helps to stabilize the solution and the free chlorine and pH measurements stay stable over time, as compared to similar compositions without the additional salts.

An aspect of the present specification discloses a method to add one or more salts during the manufacturing process. An aspect of the present specification discloses a method to add the salts after the manufacturing process. It is important to achieve free chlorine levels, PH levels, and osmolality levels of the final product specification at the time of product release.

An aspect of the present specification discloses a chemistry that balances this chemical equilibrium to allow for the simultaneous presence of hydrogen peroxide and hypochlorous acid. Notably, as the solution achieves saturation of dissolved chlorine to achieve balance with the dissolved peroxide, the solution exhibits a color change to become greyer in appearance and the formation and release of excess oxygen gas (O₂) occurs during the manufacturing process while significantly reducing the release of chlorine gas (odor) from the manufactured solution. Additionally, there is a dynamic interplay of various reactive ionic species of chlorine and oxygen, that is at least partially pH dependent for a subset of these species. Besides hypochlorous acid, hypochlorite (OCl⁻), chlorine gas (Cl₂), other stable free available chlorine species include, without limitation, superchloride (Cl₂⁻), chlorine oxide radical (ClO), peroxyhypochlorous acid (HOOCl), another reactive chlorine species (RCS), or a combination thereof. Reactive oxygen species produced by this dynamic equilibrium shift can include, without limitation, superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃), another reactive oxygen species (ROS), or a combination thereof.

In some embodiments, method of producing a solution comprising one or more stable free available chlorine species and one or more peroxides comprises first preparing a first solution comprising purified water and an appropriate trace amount of one or more peroxides. Chlorine gas is then introduced to the first solution for a first period of time to produce a first reaction solution. Without wishing to be bound by any theory, it is believed that this first reaction solution comprises approximately equimolar amounts of one or more free available chlorine species and one or more peroxide species and has a pH below 4. Once this first reaction step is completed, an appropriate trace amount of one or more peroxides is added to the first reaction solution. This peroxide addition has the effect of increasing the pH to above 9 and eliminating the detection of the one or more free available chlorine species previously produced. Chlorine gas is then again introduced to the first reaction solution for a second period of time to produce a second reaction solution. Without wishing to be bound by any theory, it is believed that this second reaction solution comprises approximately equimolar amounts of one or more free available chlorine species and one or more peroxide species and has a pH below 4. Surprisingly, repeating of the peroxide addition and chlorine gassing steps a second time resulted in significantly higher levels of one or more free available chlorine species and one or more peroxides in the resulting second reaction solution as compared to the first reaction solution, even though no free available chlorine species is detected after the second addition of the one or more peroxides. Subsequent repeats of the peroxide addition and chlorine gassing steps for a third time or more resulted in further increased levels of one or more free available chlorine species and one or more peroxides in the resulting solution. In some embodiments, the periods of time a solution is introduced with chlorine gas is successively shorter in duration. For example, in a method disclosed herein comprising two rounds of peroxide addition and chlorine gassing steps, the first period of time will be longer in duration than the second period of time. Similarly, in a method disclosed herein comprising three rounds of peroxide addition and chlorine gassing steps, the first period of time will be longer in duration than the second period of time and the second period of time will be longer in duration than the third period of time depending on the increased amount of sodium peroxide added and final desired free available chlorine in the final solution.

A method of producing a solution comprising one or more stable free available chlorine species and one or more peroxides comprises preparing a first solution comprising an appropriate amount of one or more peroxides. In some embodiments, an appropriate amount of one or more peroxides in a first solution can be, e.g., about 0.01% to about 0.2%, about 0.02% to about 0.19%, about 0.03% to about 0.18%, about 0.04% to about 0.17%, about 0.05% to about 0.16%, about 0.06% to about 0.15%, about 0.07% to about 0.14%, or about 0.08% to about 0.13% by weight of the first solution.

A method of producing a solution comprising one or more stable free available chlorine species and one or more peroxides comprises introducing a chlorine gas to a first solution for a first period of time. In some embodiments, a first period of time can be, e.g., about 30 minutes to about 40 minutes, about 30 minutes to about 45 minutes, about 30 minutes to about 50 minutes, about 30 minutes to about 55 minutes, or about 30 minutes to about 60 minutes.

A method of producing a solution comprising one or more stable free available chlorine species and one or more peroxides comprises preparing a first reaction solution comprising an appropriate amount of one or more peroxides. In some embodiments, an appropriate amount of one or more peroxides in a first reaction solution can be, e.g., about 0.001% to about 0.01%, about 0.001% to about 0.0125%, about 0.001% to about 0.015%, about 0.001% to about 0.0175%, about 0.001% to about 0.02%, about 0.001% to about 0.03%, about 0.001% to about 0.04%, about 0.0025% to about 0.01%, about 0.0025% to about 0.0125%, about 0.0025% to about 0.015%, about 0.0025% to about 0.0175%, about 0.0025% to about 0.02%, about 0.0025% to about 0.03%, about 0.0025% to about 0.04%, about 0.005% to about 0.01%, about 0.005% to about 0.0125%, about 0.005% to about 0.015%, about 0.005% to about 0.0175%, about 0.005% to about 0.02%, about 0.005% to about 0.03%, about 0.005% to about 0.04%, about 0.0075% to about 0.01%, about 0.0075% to about 0.0125%, about 0.0075% to about 0.015%, about 0.0075% to about 0.0175%, about 0.0075% to about 0.02%, about 0.0075% to about 0.03%, about 0.0075% to about 0.04%, about 0.01% to about 0.0125%, about 0.01% to about 0.015%, about 0.01% to about 0.0175%, about 0.01% to about 0.02%, about 0.01% to about 0.03%, or about 0.01% to about 0.04% by weight of the first reaction solution.

A method of producing a solution comprising one or more stable free available chlorine species and one or more peroxides comprises introducing a chlorine gas to a first reaction solution for a second period of time. In some embodiments, the second period of time of introducing a chlorine gas to a first reaction solution is of shorter duration than the first period of time of introducing a chlorine gas to a first solution. In some embodiments, a second period of time can be, e.g., about 5 minutes to about 10 minutes, about 5 minutes to about 15 minutes, about 5 minutes to about 20 minutes, about 5 minutes to about 30 minutes, about 10 minutes to about 15 minutes, about 10 minutes to about 20 minutes, about 10 minutes to about 30 minutes, about 15 minutes to about 20 minutes, about 15 minutes to about 30 minutes, or about 20 minutes to about 30 minutes.

A method of producing a solution comprising one or more stable free available chlorine species and one or more peroxides comprises preparing a second reaction solution comprising an appropriate amount of one or more peroxides. In some embodiments, an appropriate amount of one or more peroxides in a second reaction solution can be, e.g., about 0.001% to about 0.01%, about 0.001% to about 0.0125%, about 0.001% to about 0.015%, about 0.001% to about 0.0175%, about 0.001% to about 0.02%, about 0.001% to about 0.03%, about 0.001% to about 0.04%, about 0.0025% to about 0.01%, about 0.0025% to about 0.0125%, about 0.0025% to about 0.015%, about 0.0025% to about 0.0175%, about 0.0025% to about 0.02%, about 0.0025% to about 0.03%, about 0.0025% to about 0.04%, about 0.005% to about 0.01%, about 0.005% to about 0.0125%, about 0.005% to about 0.015%, about 0.005% to about 0.0175%, about 0.005% to about 0.02%, about 0.005% to about 0.03%, about 0.005% to about 0.04%, about 0.0075% to about 0.01%, about 0.0075% to about 0.0125%, about 0.0075% to about 0.015%, about 0.0075% to about 0.0175%, about 0.0075% to about 0.02%, about 0.0075% to about 0.03%, about 0.0075% to about 0.04%, about 0.01% to about 0.0125%, about 0.01% to about 0.015%, about 0.01% to about 0.0175%, about 0.01% to about 0.02%, about 0.01% to about 0.03%, or about 0.01% to about 0.04% by weight of the first reaction solution.

A method of producing a solution comprising one or more stable free available chlorine species and one or more peroxides comprises introducing a chlorine gas to a second reaction solution for a third period of time. In some embodiments, the third period of time of introducing a chlorine gas to a second reaction solution is of shorter duration than the second period of time of introducing a chlorine gas to a first reaction solution. In some embodiments, a first period of time can be, e.g., about 1 minute to about 3 minutes, about 1 minute to about 5 minutes, about 1 minute to about 7 minutes, about 1 minute to about 10 minutes, about 3 minutes to about 5 minutes, about 3 minutes to about 7 minutes, about 3 minutes to about 10 minutes, about 5 minutes to about 10 minutes, or about 7 minutes to about 10 minutes.

A method of producing a solution comprising one or more stable free available chlorine species and one or more peroxides comprising further peroxide addition and chlorine gassing steps, i.e., a fourth repeat, fifth repeat, sixth repeat, etc. will employ conditions similar to the third repeated cycle.

In some embodiments, a method disclosed herein uses a peroxide disclosed herein to stabilize free available chlorine. In some embodiments, the method includes adding a peroxide disclosed herein at the time of producing free available chlorine. In aspects of these embodiments, free available chlorine is produced using a hydrolysis method or an electrolysis method. A peroxide disclosed herein may be added as a starting ingredient to facilitate simultaneous production of free available chlorine and a peroxide disclosed herein in situ.

In some embodiments, a method disclosed herein uses sodium peroxide to stabilize free available chlorine. In some embodiments, the method includes adding sodium peroxide at the time of producing free available chlorine. In aspects of these embodiments, free available chlorine is produced using a hydrolysis method or an electrolysis method. Sodium peroxide may be added as a starting ingredient to facilitate simultaneous production of free available chlorine and hydrogen peroxide in situ.

Aspects of the present specification disclose a method of producing a composition comprising stable free available chlorine and one or more peroxides in an aqueous solution. An alternative method to achieve the above stable chemistry in equilibrium may be achieved by the combination of oxygen gas (O₂) with chlorine gas (Cl₂) in an aqueous solution with dissolved NaCl. Although not as efficient as the sodium peroxide method, the chemistry may be reproduced to equilibrium with such methods.

Aspects of the present specification disclose a method of combining stable free available chlorine with hydrogen peroxide in an aqueous solution. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. In some embodiments, a method disclosed herein uses sodium peroxide to stabilize free available chlorine. In some embodiments, the method includes adding sodium peroxide at the time of producing free available chlorine. In aspects of these embodiments, free available chlorine is produced using a hydrolysis method or an electrolysis method. Sodium peroxide may be added as a starting ingredient to facilitate simultaneous production of free available chlorine and hydrogen peroxide in situ.

Aspects of the present specification disclose a method of producing a composition comprising stable free available chlorine and one or more peroxides in an aqueous solution. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. A peroxide is a compound with the structure R—O—O—R. The O—O group in a peroxide is called the peroxide group or peroxo group. In contrast to oxide ions, the oxygen atoms in the peroxide ion have an oxidation state of −1. A peroxide includes metal peroxides where R is a metal atom. Metal peroxides may include alkali metal and alkaline earth metal atoms which form ion peroxides, zinc, cadmium, mercury atoms which form covalent peroxides; and magnesium atoms which form ion-covalent hybrid peroxides. One or more peroxides disclosed herein include hydrogen peroxide, sodium peroxide, calcium peroxide, lithium peroxide, potassium peroxide, other alkali metal peroxides, other alkali earth metal peroxides, or a combination thereof. In some embodiments, a method disclosed herein uses sodium peroxide to stabilize free available chlorine. In some embodiments, the method includes adding sodium peroxide at the time of producing free available chlorine. In aspects of these embodiments, free available chlorine is produced using a hydrolysis method or an electrolysis method. Sodium peroxide may be added as a starting ingredient to facilitate simultaneous production of free available chlorine and hydrogen peroxide in situ. Additionally, the disclosed method produces a composition further comprising one or more reactive oxygen species, including, without limitation, oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃), another reactive oxygen species (ROS), or a combination thereof.

Aspects of the present specification disclose a method of producing a composition comprising stable free available chlorine and hydrogen peroxide in an aqueous solution. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. In some embodiments, a method disclosed herein uses sodium peroxide to stabilize free available chlorine. In some embodiments, the method includes adding sodium peroxide at the time of producing free available chlorine. In aspects of these embodiments, free available chlorine is produced using a hydrolysis method or an electrolysis method. Sodium peroxide may be added as a starting ingredient to facilitate simultaneous production of free available chlorine and hydrogen peroxide in situ.

Aspects of the present specification disclose a peroxide for use in making a composition comprising one or more peroxides and stable free available chlorine. In other aspects, the present specification disclose use of a peroxide in making a composition comprising one or more peroxides and stable free available chlorine. In yet other aspects, the present specification discloses use of a peroxide in the manufacture of a composition comprising one or more peroxides and stable free available chlorine. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. One or more peroxides disclosed herein include hydrogen peroxide, sodium peroxide, calcium peroxide, lithium peroxide, potassium peroxide, other alkali metal peroxides, other alkali earth metal peroxides, or a combination thereof.

Aspects of the present specification disclose a peroxide for use in making a composition comprising hydrogen peroxide and stable free available chlorine. In other aspects, the present specification discloses use of a peroxide in making a composition comprising hydrogen peroxide and stable free available chlorine. In yet other aspects, the present specification discloses use of a peroxide in the manufacture of a composition comprising hydrogen peroxide and stable free available chlorine.

Aspects of the present specification disclose a sodium peroxide for use in making a composition comprising hydrogen peroxide and stable free available chlorine. In other aspects, the present specification disclose use of a sodium peroxide in making a composition comprising hydrogen peroxide and stable free available chlorine. In yet other aspects, the present specification discloses use of a sodium peroxide in the manufacture of a composition comprising hydrogen peroxide and stable free available chlorine.

Aspects of the present specification disclose a peroxide for use in making a composition comprising one or more peroxides and stable hypochlorous acid. In other aspects, the present specification discloses use of a peroxide in making a composition comprising one or more peroxides and stable hypochlorous acid. In yet other aspects, the present specification discloses use of a peroxide in the manufacture of a composition comprising one or more peroxides and stable hypochlorous acid. Stable free available chlorine disclosed herein may include compositions of hypochlorous acid, hypochlorite (OCl⁻), chlorine gas (Cl₂), superchloride (Cl₂⁻), chlorine oxide radical (ClO), another reactive chlorine species (RCS), or a combination thereof. One or more peroxides disclosed herein include hydrogen peroxide, sodium peroxide, calcium peroxide, lithium peroxide, potassium peroxide, other alkali metal peroxides, other alkali earth metal peroxides, or a combination thereof.

Aspects of the present specification disclose a peroxide for use in making a composition comprising hydrogen peroxide and stable hypochlorous acid. In other aspects, the present specification discloses use of a peroxide in making a composition comprising hydrogen peroxide and stable hypochlorous acid. In yet other aspects, the present specification discloses use of a peroxide in the manufacture of a composition comprising hydrogen peroxide and stable hypochlorous acid.

Aspects of the present specification disclose a sodium peroxide for use in making a composition comprising hydrogen peroxide and stable hypochlorous acid. In other aspects, the present specification discloses use of a sodium peroxide in making a composition comprising hydrogen peroxide and stable hypochlorous acid. In yet other aspects, the present specification disclose use of a sodium peroxide in the manufacture of a composition comprising hydrogen peroxide and stable hypochlorous acid.

The amount of a peroxide disclosed herein used in a method or use disclosed herein is an amount sufficient to stabilize free available chlorine or a component thereof. In aspects of these embodiments, the amount of a peroxide disclosed herein added to a method or use disclosed herein is to a final percent weight concentration of, e.g., about 0.001%, about 0.0025%, about 0.005%, about 0.0075%, about 0.01%, about 0.02%, about 0.03%, about 0.04%, about 0.05%, or about 0.1%. In other aspects of these embodiments, the amount of a peroxide disclosed herein added to a method or use disclosed herein is to a final percent weight concentration of, e.g., at least 0.001%, at least 0.0025%, at least 0.005%, at least 0.0075%, at least 0.01%, at least 0.02%, at least 0.03%, at least 0.04%, at least 0.05%, or at least 0.1%. In yet other aspects of these embodiments, the amount of a peroxide added to a method or use disclosed herein is to a final percent weight concentration of, e.g., at most 0.001%, at most 0.0025%, at most 0.005%, at most 0.7%, at most 0.01%, at most 0.02%, at most 0.03%, at most 0.04%, at most 0.05%, or as most 0.1%. In still other aspects of these embodiments, the amount of a peroxide disclosed herein added to a method or use disclosed herein is to a final percent weight concentration of, e.g., about 0.001% to about 0.002%, about 0.001% to about 0.005%, about 0.005% to about 0.01%, about 0.01% to about 0.02%, about 0.01% to about 0.03%, about 0.01% to about 0.04%, about 0.01% to about 0.05%, about 0.02% to about 0.03%, about 0.02% to about 0.04%, about 0.02% to about 0.05%, about 0.03% to about 0.04%, about 0.03% to about 0.05%, about 0.03% to about 0.05%, or about 0.05% to 0.1%.

In aspects of these embodiments, the amount of sodium peroxide added to a method or use disclosed herein is to a final percent weight concentration of, e.g., about 0.001%, about 0.0025%, about 0.005%, about 0.0075%, 0.01%, about 0.02%, about 0.03%, about 0.04%, about 0.05%, or about 0.1%. In other aspects of these embodiments, the amount of sodium peroxide added to a method or use disclosed herein is to a final percent weight concentration of, e.g., at least 0.001%, at least 0.0025%, at least 0.005%, at least 0.0075%, at least 0.01%, at least 0.02%, at least 0.03%, at least 0.04%, at least 0.05%, or at least 0.1%. In yet other aspects of these embodiments, the amount of sodium peroxide added to a method or use disclosed herein is to a final percent weight concentration of, e.g., at most 0.001%, at most 0.0025%, at most 0.005%, at most 0.7%, at most 0.01%, at most 0.02%, at most 0.03%, at most 0.04%, at most 0.05%, or as most 0.1%. In still other aspects of these embodiments, the amount of sodium peroxide added to a method or use disclosed herein is to a final percent weight concentration of, e.g., about 0.001% to about 0.002%, about 0.001% to about 0.005%, about 0.005% to about 0.01%, about 0.01% to about 0.02%, about 0.01% to about 0.03%, about 0.01% to about 0.04%, about 0.01% to about 0.05%, about 0.02% to about 0.03%, about 0.02% to about 0.04%, about 0.02% to about 0.05%, about 0.03% to about 0.04%, about 0.03% to about 0.05%, about 0.03% to about 0.05%, or about 0.05% to 0.1%.

In aspects of these embodiments, the amount of a peroxide disclosed herein added to a method or use disclosed herein is to a final percent weight concentration of, e.g., about 0.08%, about 0.09%, about 0.1%, about 011%, about 0.12%, about 0.13%, about 0.14%, about 0.15%, about 0.16%, or about 0.17%. In other aspects of these embodiments, the amount of a peroxide disclosed herein added to a method or use disclosed herein is to a final percent weight concentration of, e.g., at least 0.08%, at least 0.09%, at least 0.1%, at least 011%, at least 0.12%, at least 0.13%, at least 0.14%, at least 0.15%, at least 0.16%, or at least 0.17%. In yet other aspects of these embodiments, the amount of a peroxide added to a method or use disclosed herein is to a final percent weight concentration of, e.g., at most 0.08%, at most 0.09%, at most 0.1%, at most 011%, at most 0.12%, at most 0.13%, at most 0.14%, at most 0.15%, at most 0.16%, or at most 0.17%. In still other aspects of these embodiments, the amount of a peroxide disclosed herein added to a method or use disclosed herein is to a final percent weight concentration of, e.g., about 0.08% to about 0.09%, about 0.08% to about 0.1%, about 0.08% to about 0.11%, about 0.08% to about 0.12%, about 0.08% to about 0.13%, about 0.08% to about 0.14%, about 0.08% to about 0.15%, about 0.08% to about 0.16%, about 0.08% to about 0.17%, about 0.08% to about 0.18%, about 0.08% to about 0.19%, about 0.08% to about 0.20%, about 0.09% to about 0.1%, about 0.09% to about 0.11%, about 0.09% to about 0.12%, about 0.09% to about 0.13%, about 0.09% to about 0.14%, about 0.09% to about 0.15%, about 0.09% to about 0.16%, about 0.09% to about 0.17%, about 0.09% to about 0.18%, about 0.09% to about 0.19%, about 0.09% to about 0.20%, about 0.1% to about 0.11%, about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 0.20%, about 0.11% to about 0.12%, about 0.11% to about 0.13%, about 0.11% to about 0.14%, about 0.11% to about 0.15%, about 0.11% to about 0.16%, about 0.11% to about 0.17%, about 0.11% to about 0.18%, about 0.11% to about 0.19%, about 0.11% to about 0.20%, about 0.12% to about 0.13%, about 0.12% to about 0.14%, about 0.12% to about 0.15%, about 0.12% to about 0.16%, about 0.12% to about 0.17%, about 0.12% to about 0.18%, about 0.12% to about 0.19%, or about 0.12% to about 0.20%.

In aspects of these embodiments, the amount of a sodium peroxide disclosed herein added to a method or use disclosed herein is to a final percent weight concentration of, e.g., about 0.08%, about 0.09%, about 0.1%, about 011%, about 0.12%, about 0.13%, about 0.14%, about 0.15%, about 0.16%, or about 0.17%. In other aspects of these embodiments, the amount of a sodium peroxide disclosed herein added to a method or use disclosed herein is to a final percent weight concentration of, e.g., at least 0.08%, at least 0.09%, at least 0.1%, at least 011%, at least 0.12%, at least 0.13%, at least 0.14%, at least 0.15%, at least 0.16%, or at least 0.17%. In yet other aspects of these embodiments, the amount of sodium peroxide added to a method or use disclosed herein is to a final percent weight concentration of, e.g., at most 0.08%, at most 0.09%, at most 0.1%, at most 011%, at most 0.12%, at most 0.13%, at most 0.14%, at most 0.15%, at most 0.16%, or at most 0.17%. In still other aspects of these embodiments, the amount of a sodium peroxide disclosed herein added to a method or use disclosed herein is to a final percent weight concentration of, e.g., about 0.08% to about 0.09%, about 0.08% to about 0.1%, about 0.08% to about 0.11%, about 0.08% to about 0.12%, about 0.08% to about 0.13%, about 0.08% to about 0.14%, about 0.08% to about 0.15%, about 0.08% to about 0.16%, about 0.08% to about 0.17%, about 0.08% to about 0.18%, about 0.08% to about 0.19%, about 0.08% to about 0.20%, about 0.09% to about 0.1%, about 0.09% to about 0.11%, about 0.09% to about 0.12%, about 0.09% to about 0.13%, about 0.09% to about 0.14%, about 0.09% to about 0.15%, about 0.09% to about 0.16%, about 0.09% to about 0.17%, about 0.09% to about 0.18%, about 0.09% to about 0.19%, about 0.09% to about 0.20%, about 0.1% to about 0.11%, about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 0.20%, about 0.11% to about 0.12%, about 0.11% to about 0.13%, about 0.11% to about 0.14%, about 0.11% to about 0.15%, about 0.11% to about 0.16%, about 0.11% to about 0.17%, about 0.11% to about 0.18%, about 0.11% to about 0.19%, about 0.11% to about 0.20%, about 0.12% to about 0.13%, about 0.12% to about 0.14%, about 0.12% to about 0.15%, about 0.12% to about 0.16%, about 0.12% to about 0.17%, about 0.12% to about 0.18%, about 0.12% to about 0.19%, or about 0.12% to about 0.20%.

In aspects of these embodiments, the amount of a peroxide disclosed herein added to a method or use disclosed herein is to a final concentration of, e.g., 0.1 ppm, 0.5 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, or 1,000 ppm. In other aspects of these embodiments, the amount of a peroxide disclosed herein added to a method or use disclosed herein is to a final concentration of, e.g., at least 0.1 ppm, at least 0.5 ppm, at least 1 ppm, at least 5 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, or at least 1,000 ppm. In yet other aspects of these embodiments, the amount of a peroxide disclosed herein added to a method or use disclosed herein is to a final concentration of, e.g., at most 0.1 ppm, at most 0.5 ppm, at most 1 ppm, at most 5 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, or at most 1,000 ppm.

In still other aspects of these embodiments, the amount of a peroxide disclosed herein added to a method or use disclosed herein is to a final concentration of, e.g., about 0.1 ppm to about 25 ppm, about 0.1 ppm to about 50 ppm, about 0.1 ppm to about 75 ppm, about 0.1 ppm to about 100 ppm, about 0.1 ppm to about 125 ppm, about 0.1 ppm to about 150 ppm, about 0.1 ppm to about 175 ppm, about 0.1 ppm to about 200 ppm, about 0.1 ppm to about 225 ppm, about 0.1 ppm to about 250 ppm, about 0.1 ppm to about 275 ppm, about 0.1 ppm to about 300 ppm, about 0.1 ppm to about 325 ppm, about 0.1 ppm to about 350 ppm, about 0.1 ppm to about 375 ppm, about 0.1 ppm to about 400 ppm, about 0.1 ppm to about 425 ppm, about 0.1 ppm to about 450 ppm, about 0.1 ppm to about 475 ppm, about 0.1 ppm to about 500 ppm, about 0.5 ppm to about 25 ppm, about 0.5 ppm to about 50 ppm, about 0.5 ppm to about 75 ppm, about 0.5 ppm to about 100 ppm, about 0.5 ppm to about 125 ppm, about 0.5 ppm to about 150 ppm, about 0.5 ppm to about 175 ppm, about 0.5 ppm to about 200 ppm, about 0.5 ppm to about 225 ppm, about 0.5 ppm to about 250 ppm, about 0.5 ppm to about 275 ppm, about 0.5 ppm to about 300 ppm, about 0.5 ppm to about 325 ppm, about 0.5 ppm to about 350 ppm, about 0.5 ppm to about 375 ppm, about 0.5 ppm to about 400 ppm, about 0.5 ppm to about 425 ppm, about 0.5 ppm to about 450 ppm, about 0.5 ppm to about 475 ppm, about 0.5 ppm to about 500 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 5 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In still other aspects of these embodiments, the amount of a peroxide disclosed herein added to a method or use disclosed herein is to a final concentration of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, or about 1,000 ppm to about 2,000 ppm.

In aspects of these embodiments, the amount of sodium peroxide added to a method or use disclosed herein is to a final concentration of, e.g., 0.1 ppm, 0.5 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, or 1,000 ppm. In other aspects of these embodiments, the amount of sodium peroxide added to a method or use disclosed herein is to a final concentration of, e.g., at least 0.1 ppm, at least 0.5 ppm, at least 1 ppm, at least 5 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, or at least 1,000 ppm. In yet other aspects of these embodiments, the amount of sodium peroxide added to a method or use disclosed herein is to a final concentration of, e.g., at most 0.1 ppm, at most 0.5 ppm, at most 1 ppm, at most 5 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, or at most 1,000 ppm.

In still other aspects of these embodiments, the amount of sodium peroxide added to a method or use disclosed herein is to a final concentration of, e.g., about 0.1 ppm to about 25 ppm, about 0.1 ppm to about 50 ppm, about 0.1 ppm to about 75 ppm, about 0.1 ppm to about 100 ppm, about 0.1 ppm to about 125 ppm, about 0.1 ppm to about 150 ppm, about 0.1 ppm to about 175 ppm, about 0.1 ppm to about 200 ppm, about 0.1 ppm to about 225 ppm, about 0.1 ppm to about 250 ppm, about 0.1 ppm to about 275 ppm, about 0.1 ppm to about 300 ppm, about 0.1 ppm to about 325 ppm, about 0.1 ppm to about 350 ppm, about 0.1 ppm to about 375 ppm, about 0.1 ppm to about 400 ppm, about 0.1 ppm to about 425 ppm, about 0.1 ppm to about 450 ppm, about 0.1 ppm to about 475 ppm, about 0.1 ppm to about 500 ppm, about 0.5 ppm to about 25 ppm, about 0.5 ppm to about 50 ppm, about 0.5 ppm to about 75 ppm, about 0.5 ppm to about 100 ppm, about 0.5 ppm to about 125 ppm, about 0.5 ppm to about 150 ppm, about 0.5 ppm to about 175 ppm, about 0.5 ppm to about 200 ppm, about 0.5 ppm to about 225 ppm, about 0.5 ppm to about 250 ppm, about 0.5 ppm to about 275 ppm, about 0.5 ppm to about 300 ppm, about 0.5 ppm to about 325 ppm, about 0.5 ppm to about 350 ppm, about 0.5 ppm to about 375 ppm, about 0.5 ppm to about 400 ppm, about 0.5 ppm to about 425 ppm, about 0.5 ppm to about 450 ppm, about 0.5 ppm to about 475 ppm, about 0.5 ppm to about 500 ppm, about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 5 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In still other aspects of these embodiments, the amount of sodium peroxide added to a method or use disclosed herein is to a final concentration of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, or about 1,000 ppm to about 2,000 ppm.

A method of manufacture disclosed herein produces a composition comprising one or more peroxides and a stable free available chlorine. One or more peroxides disclosed herein include hydrogen peroxide, sodium peroxide, calcium peroxide, lithium peroxide, potassium peroxide, other alkali metal peroxides, other alkali earth metal peroxides, or a combination thereof. Stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof. Additionally, the disclosed method produces a composition further comprising one or more reactive oxygen species, including, without limitation, oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃⁻), another reactive oxygen species (ROS), or a combination thereof.

A composition manufactured according to a method or use disclosed herein may comprise one or more peroxides disclosed herein. In some embodiments, a composition manufactured according to a method or use disclosed herein comprises an amount of one or more peroxides that provides a desired beneficial effect to a composition manufactured according to a method or use disclosed herein. In some embodiments, when a composition manufactured according to a method or use disclosed herein comprising one or more peroxides is formulated to be used in an undiluted state, the amount of peroxide used is an amount effective to clean, disinfect, sanitize, and/or sterilize. In some embodiments, when a composition manufactured according to a method or use disclosed herein comprising one or more peroxides is formulated to be used in an undiluted state, the amount of peroxide used is a microbiostatic effective amount. A microbiostatic effective amount of one or more peroxides is an amount that restrains, prohibits, or otherwise prevents microbial population growth without eradicating the microbes. In an aspect of this embodiment, a microbiostatic effective amount of one or more peroxides is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising one or more peroxides will be diluted, the amount of peroxide used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises one or more peroxides disclosed herein each in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises one or more peroxides disclosed herein each in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises one or more peroxides disclosed herein each in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises one or more peroxides disclosed herein each in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises one or more peroxides disclosed herein each in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises one or more peroxides disclosed herein each in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of these embodiments, a composition manufactured according to a method or use disclosed herein comprises one or more peroxides disclosed herein each in an amount of, e.g., about 0.08%, about 0.09%, about 0.1%, about 011%, about 0.12%, about 0.13%, about 0.14%, about 0.15%, about 0.16%, about 0.17%, about 0.18%, about 0.19%, or about 0.20%. In other aspects of these embodiments, a composition manufactured according to a method or use disclosed herein comprises one or more peroxides disclosed herein each in an amount of, e.g., at least 0.08%, at least 0.09%, at least 0.1%, at least 011%, at least 0.12%, at least 0.13%, at least 0.14%, at least 0.15%, at least 0.16%, at least 0.17%, at least 0.18%, at least 0.19%, or at least 0.20%. In yet other aspects of these embodiments, a composition manufactured according to a method or use disclosed herein comprises one or more peroxides disclosed herein each in an amount of, e.g., at most 0.08%, at most 0.09%, at most 0.1%, at most 011%, at most 0.12%, at most 0.13%, at most 0.14%, at most 0.15%, at most 0.16%, at most 0.17%, at most 0.18%, at most 0.19%, or at most 0.20%. In still other aspects of these embodiments, a composition manufactured according to a method or use disclosed herein comprises one or more peroxides disclosed herein each in an amount of, e.g., about 0.08% to about 0.09%, about 0.08% to about 0.1%, about 0.08% to about 0.11%, about 0.08% to about 0.12%, about 0.08% to about 0.13%, about 0.08% to about 0.14%, about 0.08% to about 0.15%, about 0.08% to about 0.16%, about 0.08% to about 0.17%, about 0.08% to about 0.18%, about 0.08% to about 0.19%, about 0.08% to about 0.20%, about 0.09% to about 0.1%, about 0.09% to about 0.11%, about 0.09% to about 0.12%, about 0.09% to about 0.13%, about 0.09% to about 0.14%, about 0.09% to about 0.15%, about 0.09% to about 0.16%, about 0.09% to about 0.17%, about 0.09% to about 0.18%, about 0.09% to about 0.19%, about 0.09% to about 0.20%, about 0.1% to about 0.11%, about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 0.20%, about 0.11% to about 0.12%, about 0.11% to about 0.13%, about 0.11% to about 0.14%, about 0.11% to about 0.15%, about 0.11% to about 0.16%, about 0.11% to about 0.17%, about 0.11% to about 0.18%, about 0.11% to about 0.19%, about 0.11% to about 0.20%, about 0.12% to about 0.13%, about 0.12% to about 0.14%, about 0.12% to about 0.15%, about 0.12% to about 0.16%, about 0.12% to about 0.17%, about 0.12% to about 0.18%, about 0.12% to about 0.19%, or about 0.12% to about 0.20%.

A composition manufactured according to a method or use disclosed herein may comprise a hydrogen peroxide. The simplest peroxide, hydrogen peroxide, has the chemical formula H₂O₂, while its molecular formula is written as HOOH. As shown in Formula I, hydrogen peroxide is a simple, nonplanar molecule with a twisted C2 symmetry and has molar mass is 34.01 g/mol. Although the O—O bond is a single bond, the molecule has a relatively high rotational barrier of 2460 cm⁻¹ (29.45 KJ/mol).

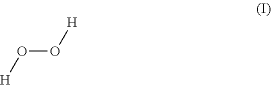

Hydrogen peroxide is a very pale blue liquid in its pure form, slightly more viscous than water. Hydrogen peroxide is manufactured almost exclusively by the anthraquinone process. It begins with the reduction of an anthraquinone (such as 2-ethylanthraquinone or the 2-amyl derivative) to the corresponding anthrahydroquinone, typically by hydrogenation on a palladium catalyst. In the presence of oxygen, the anthrahydroquinone then undergoes autoxidation: the labile hydrogen atoms of the hydroxy groups transfer to the oxygen molecule, to give hydrogen peroxide and regenerate the anthraquinone. Most commercial processes achieve oxidation by bubbling compressed air through a solution of the anthrahydroquinone, with the hydrogen peroxide then extracted from the solution and the anthraquinone recycled back for successive cycles of hydrogenation and oxidation. As an alternative, sodium peroxide (Na₂O₂) can undergo hydrolysis in the presence of water to yield hydrogen peroxide and sodium hydroxide (NaOH).

In some embodiments, a composition manufactured according to a method or use disclosed herein comprises an amount of hydrogen peroxide that provides a desired beneficial effect to a composition manufactured according to a method or use disclosed herein. In some embodiments, when a composition manufactured according to a method or use disclosed herein comprising hydrogen peroxide is formulated to be used in an undiluted state, the amount of hydrogen peroxide used is an amount effective to clean, disinfect, sanitize, and/or sterilize. In some embodiments, when a composition manufactured according to a method or use disclosed herein comprising hydrogen peroxide is formulated to be used in an undiluted state, the amount of hydrogen peroxide used is a microbiostatic effective amount. A microbiostatic effective amount of hydrogen peroxide is an amount that restrains, prohibits, or otherwise prevents microbial population growth without eradicating the microbes. In an aspect of this embodiment, a microbiostatic effective amount of hydrogen peroxide is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising hydrogen peroxide will be diluted, the amount of hydrogen peroxide used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises hydrogen peroxide in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises hydrogen peroxide in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises hydrogen peroxide in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises hydrogen peroxide in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises hydrogen peroxide in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises hydrogen peroxide in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

A composition manufactured according to a method or use disclosed herein may comprise a sodium peroxide. In some embodiments, a composition manufactured according to a method or use disclosed herein comprises an amount of sodium peroxide that provides a desired beneficial effect to a composition manufactured according to a method or use disclosed herein. In some embodiments, when a composition manufactured according to a method or use disclosed herein comprising sodium peroxide is formulated to be used in an undiluted state, the amount of sodium peroxide used is an amount effective to clean, disinfect, sanitize, and/or sterilize. In some embodiments, when a composition manufactured according to a method or use disclosed herein comprising sodium peroxide is formulated to be used in an undiluted state, the amount of sodium peroxide used is a microbiostatic effective amount. A microbiostatic effective amount of sodium peroxide is an amount that restrains, prohibits, or otherwise prevents microbial population growth without eradicating the microbes. In an aspect of this embodiment, a microbiostatic effective amount of sodium peroxide is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising sodium peroxide will be diluted, the amount of sodium peroxide used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises sodium peroxide in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises sodium peroxide in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises sodium peroxide in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises sodium peroxide in an amount of, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises sodium peroxide in an amount of, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises sodium peroxide in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of these embodiments, a composition manufactured according to a method or use disclosed herein comprises sodium peroxide in an amount of, e.g., about 0.08%, about 0.09%, about 0.1%, about 011%, about 0.12%, about 0.13%, about 0.14%, about 0.15%, about 0.16%, about 0.17%, about 0.18%, about 0.19%, or about 0.20%. In other aspects of these embodiments, a composition manufactured according to a method or use disclosed herein comprises sodium peroxide in an amount of, e.g., at least 0.08%, at least 0.09%, at least 0.1%, at least 011%, at least 0.12%, at least 0.13%, at least 0.14%, at least 0.15%, at least 0.16%, at least 0.17%, at least 0.18%, at least 0.19%, or at least 0.20%. In yet other aspects of these embodiments, a composition manufactured according to a method or use disclosed herein comprises sodium peroxide in an amount of, e.g., at most 0.08%, at most 0.09%, at most 0.1%, at most 011%, at most 0.12%, at most 0.13%, at most 0.14%, at most 0.15%, at most 0.16%, at most 0.17%, at most 0.18%, at most 0.19%, or at most 0.20%. In still other aspects of these embodiments, a composition manufactured according to a method or use disclosed herein comprises sodium peroxide in an amount of, e.g., about 0.08% to about 0.09%, about 0.08% to about 0.1%, about 0.08% to about 0.11%, about 0.08% to about 0.12%, about 0.08% to about 0.13%, about 0.08% to about 0.14%, about 0.08% to about 0.15%, about 0.08% to about 0.16%, about 0.08% to about 0.17%, about 0.08% to about 0.18%, about 0.08% to about 0.19%, about 0.08% to about 0.20%, about 0.09% to about 0.1%, about 0.09% to about 0.11%, about 0.09% to about 0.12%, about 0.09% to about 0.13%, about 0.09% to about 0.14%, about 0.09% to about 0.15%, about 0.09% to about 0.16%, about 0.09% to about 0.17%, about 0.09% to about 0.18%, about 0.09% to about 0.19%, about 0.09% to about 0.20%, about 0.1% to about 0.11%, about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 0.20%, about 0.11% to about 0.12%, about 0.11% to about 0.13%, about 0.11% to about 0.14%, about 0.11% to about 0.15%, about 0.11% to about 0.16%, about 0.11% to about 0.17%, about 0.11% to about 0.18%, about 0.11% to about 0.19%, about 0.11% to about 0.20%, about 0.12% to about 0.13%, about 0.12% to about 0.14%, about 0.12% to about 0.15%, about 0.12% to about 0.16%, about 0.12% to about 0.17%, about 0.12% to about 0.18%, about 0.12% to about 0.19%, or about 0.12% to about 0.20%.

A composition manufactured according to a method or use disclosed herein may comprise a stable free available chlorine. Stable free available chlorine can be produced using hydrolysis and electrolysis methods disclosed herein. Hydrolysis involved the dissolution of chlorine gas into an aqueous solution yielding H+, Cl—, and HOCl. Electrolysis involves exposure of a saline solution (H₂O and NaCl) to anode and cathode powered electrodes and results in a release of H₂ gas at the cathode, Cl₂ gas at the anode and production of aqueous NaOH. Cl₂ gas is free to hydrolyze to produce HOCl. Thus, stable free available chlorine disclosed herein may include compositions of hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

The chlorine amount may be measured by methods known in the art, such as a DPD colorimeter method (Lamotte Company, Chestertown, Md.), a UV spectrophotometry method, or other known methods established by the Environmental Protection Agency. In the DPD colorimeter method, a yellow color is formed by the reaction of free chlorine with N, N-diethyl-p-phenylenediamine (DPD) and the intensity is measured with a calibrated calorimeter that provides the output in parts per million. Further addition of potassium iodide turns the solution a pink color to provide the total chlorine value.

In some embodiments, a composition manufactured according to a method or use disclosed herein comprises an amount of stable free available chlorine that provides a desired beneficial effect to a composition disclosed herein. In some embodiments, when a composition manufactured according to a method or use disclosed herein comprising stable free available chlorine is formulated to be used in an undiluted state, the amount of stable free available chlorine used is an amount effective to clean, disinfect, sanitize, and/or sterilize. In some embodiments, when a composition manufactured according to a method or use disclosed herein comprising stable free available chlorine is formulated to be used in an undiluted state, the amount of stable free available chlorine used is a microbiostatic effective amount. A microbiostatic effective amount of stable free available chlorine is an amount that restrains, prohibits, or otherwise prevents microbial population growth without eradicating the microbes. In an aspect of this embodiment, a microbiostatic effective amount of stable free available chlorine is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising stable free available chlorine will be diluted, the amount of stable free available chlorine used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises stable free available chlorine in an amount of, e.g., 0.01 ppm, 0.025 ppm, 0.05 ppm, 0.075 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises stable free available chlorine in an amount of, e.g., at least 0.01 ppm, at least 0.025 ppm, at least 0.05 ppm, at least 0.075 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises stable free available chlorine in an amount of, e.g., at most 0.01 ppm, at most 0.025 ppm, at most 0.05 ppm, at most 0.075 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises stable free available chlorine in an amount of between, e.g., about 0.01 ppm to about 0.025 ppm, about 0.01 ppm to about 0.05 ppm, about 0.01 ppm to about 0.075 ppm, about 0.01 ppm to about 0.1 ppm, about 0.01 ppm to about 0.25 ppm, about 0.01 ppm to about 0.5 ppm, about 0.01 ppm to about 0.75 ppm, about 0.01 ppm to about 1 ppm, about 0.01 ppm to about 2 ppm, about 0.01 ppm to about 3 ppm, about 0.01 ppm to about 4 ppm, about 0.01 ppm to about 5 ppm, about 0.025 ppm to about 0.075 ppm, about 0.025 ppm to about 0.1 ppm, about 0.025 ppm to about 0.25 ppm, about 0.025 ppm to about 0.5 ppm, about 0.025 ppm to about 0.75 ppm, about 0.025 ppm to about 1 ppm, about 0.025 ppm to about 2 ppm, about 0.025 ppm to about 3 ppm, about 0.025 ppm to about 4 ppm, about 0.025 ppm to about 5 ppm, about 0.05 ppm to about 0.075 ppm, about 0.05 ppm to about 0.1 ppm, about 0.05 ppm to about 0.25 ppm, about 0.05 ppm to about 0.5 ppm, about 0.05 ppm to about 0.75 ppm, about 0.05 ppm to about 1 ppm, about 0.05 ppm to about 2 ppm, about 0.05 ppm to about 3 ppm, about 0.05 ppm to about 4 ppm, about 0.05 ppm to about 5 ppm, about 0.075 ppm to about 0.1 ppm, about 0.075 ppm to about 0.25 ppm, about 0.075 ppm to about 0.5 ppm, about 0.075 ppm to about 0.75 ppm, about 0.075 ppm to about 1 ppm, about 0.075 ppm to about 2 ppm, about 0.075 ppm to about 3 ppm, about 0.075 ppm to about 4 ppm, about 0.075 ppm to about 5 ppm, about 0.1 ppm to about 0.25 ppm, about 0.1 ppm to about 0.5 ppm, about 0.1 ppm to about 0.75 ppm, about 0.1 ppm to about 1 ppm, about 0.1 ppm to about 2 ppm, about 0.1 ppm to about 3 ppm, about 0.1 ppm to about 4 ppm, or about 0.1 ppm to about 5 ppm.

In still other aspects of this embodiment, a composition manufactured according to a method or use disclosed herein comprises stable free available chlorine in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises stable free available chlorine in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 125 ppm to about 175 ppm, about 125 ppm to about 200 ppm, about 125 ppm to about 225 ppm, about 125 ppm to about 250 ppm, about 125 ppm to about 275 ppm, about 125 ppm to about 300 ppm, about 125 ppm to about 325 ppm, about 125 ppm to about 350 ppm, about 125 ppm to about 375 ppm, about 125 ppm to about 400 ppm, about 125 ppm to about 425 ppm, about 125 ppm to about 450 ppm, about 125 ppm to about 475 ppm, about 125 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises stable free available chlorine in an amount of, e.g., about 10 ppm to about 400 ppm, about 10 ppm to about 500 ppm, about 10 ppm to about 600 ppm, about 10 ppm to about 700 ppm, about 10 ppm to about 800 ppm, about 10 ppm to about 900 ppm, about 10 ppm to about 1,000 ppm, about 25 ppm to about 400 ppm, about 25 ppm to about 500 ppm, about 25 ppm to about 600 ppm, about 25 ppm to about 700 ppm, about 25 ppm to about 800 ppm, about 25 ppm to about 900 ppm, about 25 ppm to about 1,000 ppm, about 50 ppm to about 400 ppm, about 50 ppm to about 500 ppm, about 50 ppm to about 600 ppm, about 50 ppm to about 700 ppm, about 50 ppm to about 800 ppm, about 50 ppm to about 900 ppm, about 50 ppm to about 1,000 ppm, about 75 ppm to about 400 ppm, about 75 ppm to about 500 ppm, about 75 ppm to about 600 ppm, about 75 ppm to about 700 ppm, about 75 ppm to about 800 ppm, about 75 ppm to about 900 ppm, about 75 ppm to about 1,000 ppm, about 100 ppm to about 400 ppm, about 100 ppm to about 500 ppm, about 100 ppm to about 600 ppm, about 100 ppm to about 700 ppm, about 100 ppm to about 800 ppm, about 100 ppm to about 900 ppm, about 100 ppm to about 1,000 ppm, about 125 ppm to about 400 ppm, about 125 ppm to about 500 ppm, about 125 ppm to about 600 ppm, about 125 ppm to about 700 ppm, about 125 ppm to about 800 ppm, about 125 ppm to about 900 ppm, or about 125 ppm to about 1,000 ppm.

In other aspects of this embodiment, a composition manufactured according to a method or use disclosed herein comprises stable free available chlorine in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition manufactured according to a method or use disclosed herein comprises stable free available chlorine in an amount of, e.g., about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition manufactured according to a method or use disclosed herein comprises stable free available chlorine in an amount of, e.g., at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition manufactured according to a method or use disclosed herein comprises stable free available chlorine in an amount of, e.g., at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition manufactured according to a method or use disclosed herein comprises stable free available chlorine in an amount of, e.g., about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

A composition manufactured according to a method or use disclosed herein may comprise a stable hypochlorous acid. A weak acid, the chemical formula of a stable hypochlorous acid is HOCl, while its molecular formula is written as HClO. As shown in Formula II, a stable hypochlorous acid is a simple molecule with the central oxygen connected to chlorine and hydrogen atoms through single bonds and has molar mass is 52.46 g/mol.

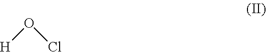

A stable hypochlorous acid is a colorless solution, and its exact physical properties are variable, depending on the concentration of hypochlorous in solution, pH, temperature, and other factors. A stable hypochlorous acid readily reacts with a variety of organic molecules, biomolecules, and other compounds.

A stable hypochlorous acid is in equilibrium with hypochlorite ions (OCl⁻) and dissolved chlorine gas (Cl₂). The extent of the equilibrium is determined predominately by the salt concentration and pH of the solution. Temperature also impacts the ratio of the free chlorine component. Therefore, both FAC and pH need to be known to understand the amount of chlorine present as hypochlorous acid. In general, when the pH range is about 4.0 to about 5.6, approximately 100% of the available chlorine is present as HOCl. As the pH is lowered below about 4, there is an increase in dissolved chlorine gas (Cl₂). Thus, at a pH of about 3, about 90% of the available chlorine is present as a stable hypochlorous acid, at a pH of about 2, about 75% of the available chlorine is present as a stable hypochlorous acid, at a pH of about 1.5, about 50% of the available chlorine is present as a stable hypochlorous acid, while at a pH of about 1, about 25% of the available chlorine is present as a stable hypochlorous acid. As the pH is increase above about 5.6, there is an increase in hypochlorite ions (OCl⁻). Thus, at a pH a pH of about 6.5, about 90% of the available chlorine is present as a stable hypochlorous acid, at a pH of about 7, about 75% of the available chlorine is present as a stable hypochlorous acid, at a pH of about 7.5, about 50% of the available chlorine is present as a stable hypochlorous acid, while at a pH of about 8, about 25% of the available chlorine is present as a stable hypochlorous acid.

In some embodiments, a composition manufactured according to a method or use disclosed herein comprises an amount of a stable hypochlorous acid that provides a desired beneficial effect to a composition manufactured according to a method or use disclosed herein. In some embodiments, when a composition manufactured according to a method or use disclosed herein comprising stable hypochlorous acid is formulated to be used in an undiluted state, the amount of stable hypochlorous acid used is an amount effective to clean, disinfect, sanitize, and/or sterilize. In some embodiments, when a composition manufactured according to a method or use disclosed herein comprising stable hypochlorous acid is formulated to be used in an undiluted state, the amount of stable hypochlorous acid used is a microbiostatic effective amount. A microbiostatic effective amount of stable hypochlorous acid is an amount that restrains, prohibits, or otherwise prevents microbial population growth without eradicating the microbes. In an aspect of this embodiment, a microbiostatic effective amount of stable hypochlorous acid is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising stable hypochlorous acid will be diluted, the amount of stable hypochlorous acid used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises a stable hypochlorous acid in an amount of, e.g., 0.01 ppm, 0.25 ppm, 0.05 ppm, 0.075, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises a stable hypochlorous acid in an amount of, e.g., at least 0.01 ppm, at least 0.25 ppm, at least 0.05 ppm, at least 0.075, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises a stable hypochlorous acid in an amount of, e.g., at most 0.01 ppm, at most 0.25 ppm, at most 0.05 ppm, at most 0.075, at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises stable hypochlorous acid in an amount of between, e.g., about 0.01 ppm to about 0.025 ppm, about 0.01 ppm to about 0.05 ppm, about 0.01 ppm to about 0.075 ppm, about 0.01 ppm to about 0.1 ppm, about 0.01 ppm to about 0.25 ppm, about 0.01 ppm to about 0.5 ppm, about 0.01 ppm to about 0.75 ppm, about 0.01 ppm to about 1 ppm, about 0.01 ppm to about 2 ppm, about 0.01 ppm to about 3 ppm, about 0.01 ppm to about 4 ppm, about 0.01 ppm to about 5 ppm, about 0.025 ppm to about 0.075 ppm, about 0.025 ppm to about 0.1 ppm, about 0.025 ppm to about 0.25 ppm, about 0.025 ppm to about 0.5 ppm, about 0.025 ppm to about 0.75 ppm, about 0.025 ppm to about 1 ppm, about 0.025 ppm to about 2 ppm, about 0.025 ppm to about 3 ppm, about 0.025 ppm to about 4 ppm, about 0.025 ppm to about 5 ppm, about 0.05 ppm to about 0.075 ppm, about 0.05 ppm to about 0.1 ppm, about 0.05 ppm to about 0.25 ppm, about 0.05 ppm to about 0.5 ppm, about 0.05 ppm to about 0.75 ppm, about 0.05 ppm to about 1 ppm, about 0.05 ppm to about 2 ppm, about 0.05 ppm to about 3 ppm, about 0.05 ppm to about 4 ppm, about 0.05 ppm to about 5 ppm, about 0.075 ppm to about 0.1 ppm, about 0.075 ppm to about 0.25 ppm, about 0.075 ppm to about 0.5 ppm, about 0.075 ppm to about 0.75 ppm, about 0.075 ppm to about 1 ppm, about 0.075 ppm to about 2 ppm, about 0.075 ppm to about 3 ppm, about 0.075 ppm to about 4 ppm, about 0.075 ppm to about 5 ppm, about 0.1 ppm to about 0.25 ppm, about 0.1 ppm to about 0.5 ppm, about 0.1 ppm to about 0.75 ppm, about 0.1 ppm to about 1 ppm, about 0.1 ppm to about 2 ppm, about 0.1 ppm to about 3 ppm, about 0.1 ppm to about 4 ppm, or about 0.1 ppm to about 5 ppm.

In still other aspects of this embodiment, a composition manufactured according to a method or use disclosed herein comprises a stable hypochlorous acid in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises a stable hypochlorous acid in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 125 ppm to about 175 ppm, about 125 ppm to about 200 ppm, about 125 ppm to about 225 ppm, about 125 ppm to about 250 ppm, about 125 ppm to about 275 ppm, about 125 ppm to about 300 ppm, about 125 ppm to about 325 ppm, about 125 ppm to about 350 ppm, about 125 ppm to about 375 ppm, about 125 ppm to about 400 ppm, about 125 ppm to about 425 ppm, about 125 ppm to about 450 ppm, about 125 ppm to about 475 ppm, about 125 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises stable hypochlorous acid in an amount of, e.g., about 10 ppm to about 400 ppm, about 10 ppm to about 500 ppm, about 10 ppm to about 600 ppm, about 10 ppm to about 700 ppm, about 10 ppm to about 800 ppm, about 10 ppm to about 900 ppm, about 10 ppm to about 1,000 ppm, about 25 ppm to about 400 ppm, about 25 ppm to about 500 ppm, about 25 ppm to about 600 ppm, about 25 ppm to about 700 ppm, about 25 ppm to about 800 ppm, about 25 ppm to about 900 ppm, about 25 ppm to about 1,000 ppm, about 50 ppm to about 400 ppm, about 50 ppm to about 500 ppm, about 50 ppm to about 600 ppm, about 50 ppm to about 700 ppm, about 50 ppm to about 800 ppm, about 50 ppm to about 900 ppm, about 50 ppm to about 1,000 ppm, about 75 ppm to about 400 ppm, about 75 ppm to about 500 ppm, about 75 ppm to about 600 ppm, about 75 ppm to about 700 ppm, about 75 ppm to about 800 ppm, about 75 ppm to about 900 ppm, about 75 ppm to about 1,000 ppm, about 100 ppm to about 400 ppm, about 100 ppm to about 500 ppm, about 100 ppm to about 600 ppm, about 100 ppm to about 700 ppm, about 100 ppm to about 800 ppm, about 100 ppm to about 900 ppm, about 100 ppm to about 1,000 ppm, about 125 ppm to about 400 ppm, about 125 ppm to about 500 ppm, about 125 ppm to about 600 ppm, about 125 ppm to about 700 ppm, about 125 ppm to about 800 ppm, about 125 ppm to about 900 ppm, or about 125 ppm to about 1,000 ppm.

In other aspects of this embodiment, a composition manufactured according to a method or use disclosed herein comprises a stable hypochlorous acid in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition manufactured according to a method or use disclosed herein comprises a stable hypochlorous acid in an amount of, e.g., about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition manufactured according to a method or use disclosed herein comprises a stable hypochlorous acid in an amount of, e.g., at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition manufactured according to a method or use disclosed herein comprises a stable hypochlorous acid in an amount of, e.g., at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition manufactured according to a method or use disclosed herein comprises a stable hypochlorous acid in an amount of, e.g., about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

A composition manufactured according to a method or use disclosed herein may comprise a stable hypochlorite (also referred to as stable hypochlorite ions). In some embodiments, a composition manufactured according to a method or use disclosed herein comprises an amount of a stable hypochlorite that provides a desired beneficial effect to a composition manufactured according to a method or use disclosed herein. In some embodiments, when a composition manufactured according to a method or use disclosed herein comprising stable hypochlorite is formulated to be used in an undiluted state, the amount of stable hypochlorite used is an amount effective to clean, disinfect, sanitize, and/or sterilize. In some embodiments, when a composition manufactured according to a method or use disclosed herein comprising stable hypochlorite is formulated to be used in an undiluted state, the amount of stable hypochlorite used is a microbiostatic effective amount. A microbiostatic effective amount of stable hypochlorite is an amount that restrains, prohibits, or otherwise prevents microbial population growth without eradicating the microbes. In an aspect of this embodiment, a microbiostatic effective amount of stable hypochlorite is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising stable hypochlorite will be diluted, the amount of stable hypochlorite used in such concentrated formulations can be any amount that does not cause precipitation of components in the composition disclosed herein or otherwise create unwanted effects that make the composition inoperable.

In aspects of this embodiment, a composition manufactured according to a method or use disclosed herein comprises a stable hypochlorite in an amount of, e.g., 0.01 ppm, 0.025 ppm, 0.05 ppm, 0.075 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises a stable hypochlorite in an amount of, e.g., at least 0.01 ppm, at least 0.025 ppm, at least 0.05 ppm, at least 0.075 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises a stable hypochlorite in an amount of, e.g., at most 0.01 ppm, at most 0.025 ppm, at most 0.05 ppm, at most 0.075 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises a stable hypochlorite in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises a stable hypochlorite in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises a stable hypochlorite in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition manufactured according to a method or use disclosed herein comprises a stable hypochlorite in an amount of, e.g., about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition manufactured according to a method or use disclosed herein comprises a stable hypochlorite in an amount of, e.g., at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition manufactured according to a method or use disclosed herein comprises a stable hypochlorite in an amount of, e.g., at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition manufactured according to a method or use disclosed herein comprises a stable hypochlorite in an amount of, e.g., about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In some embodiments, a method or use disclosed herein stabilizes free available chlorine in a manner that makes its detection and release delayed. In some embodiments, a method or use disclosed herein produces intermediary or other compounds that aid in the stabilization of free available chlorine.

In some embodiments, a method or use disclosed herein stabilizes hypochlorous acid in a manner that makes its detection and release delayed. In some embodiments, a method or use disclosed herein produces intermediary or other compounds that aid in the stabilization of hypochlorous acid.

In an embodiment, a method or use disclosed herein produces an odorless composition disclosed herein, one that masks, neutralizes, or otherwise eliminates the smell of chlorine in a solution containing stable free available chlorine.

A composition manufactured according to a method or use disclosed herein may comprise one or more reactive oxygen species. A reactive oxygen species, includes, without limitation, oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃), another reactive oxygen species (ROS), or a combination thereof.

In aspects of this embodiment, a composition manufactured according to a method or use disclosed herein comprises one or more reactive oxygen species each in an amount of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition manufactured according to a method or use disclosed herein comprises one or more reactive oxygen species each in an amount of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition manufactured according to a method or use disclosed herein comprises one or more reactive oxygen species each in an amount of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition manufactured according to a method or use disclosed herein comprises one or more reactive oxygen species each in an amount of between, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition manufactured according to a method or use disclosed herein comprises one or more reactive oxygen species each in an amount of between, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition manufactured according to a method or use disclosed herein comprises one or more reactive oxygen species each in an amount of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

A method of manufacture disclosed herein produces a composition comprising a peroxide and a stable free available chlorine disclosed herein. Such a composition is useful in the methods disclosed herein. In addition, a composition manufactured according to a method or use disclosed herein can be further modified by adding additional components. Such modified compositions are also useful in the methods disclosed herein.

In some embodiments, a method of manufacture disclosed herein comprises adding sodium peroxide to purified water contained in a vessel to prepare an aqueous solution and introducing chlorine gas to the aqueous solution under hydrolysis conditions for a first period of time to produce a first reaction solution comprising one or more stable free available chlorine species and one or more peroxides. A method of manufacture disclosed herein can further comprise degassing the first reaction solution for a degassing period of time. Optionally, sodium phosphate and/or sodium chloride can be added to an aqueous solution disclosed herein. Optionally, a method of manufacture disclosed herein further comprises adding sodium chloride to the first reaction solution to adjust the osmolality to a physiological compatible osmolality. Hydrolysis of the chlorine gas for a for a first period of time can be any time suitable to produce the desired amount of one or more stable free available chlorine species and one or more peroxides. The resulting first reaction solution produces a composition comprising one or more stable free available chlorine species and one or more peroxides that are stably maintained within the composition.

In some embodiments, a method of manufacture disclosed herein comprises adding about 0.02% (w/w) to about 0.24% (w/w) of sodium peroxide to purified water contained in a vessel to prepare an aqueous solution, and introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides. The resulting first reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w).

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides, and degassing the first reaction solution for 24 hours to 72 hours. The resulting degassed first reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w).

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides, degassing the first reaction solution for 24 hours to 72 hours, and adding about 0.5% to about 1.0% of sodium chloride to the degassed first reaction solution to adjust the osmolality to a range of about 275 mOsm/kg to about 295 mOsm/kg. The resulting osmolarity adjusted, degassed, first reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w). In aspects of these embodiments, the amount of sodium chloride added to the composition is about 0.6% to about 0.9%, or about 0.7% to about 0.8%.

In some embodiments, a method of manufacture disclosed herein comprises adding sodium peroxide to purified water contained in a sealed vessel to prepare an aqueous solution, introducing chlorine gas to the aqueous solution under hydrolysis conditions for a first period of time to produce a first reaction solution comprising one or more stable free available chlorine species and one or more peroxides, adding sodium peroxide to the first reaction solution, and introducing chlorine gas to the first reaction solution under hydrolysis conditions for a second period of time to produce a second reaction solution comprising one or more stable free available chlorine species and one or more peroxides. A method of manufacture disclosed herein can further comprise degassing the second reaction solution for a degassing period of time. Optionally, sodium phosphate and/or sodium chloride can be added to an aqueous solution disclosed herein. Optionally, a method of manufacture disclosed herein further comprises adding sodium chloride to the second reaction solution to adjust the osmolality to a physiological compatible osmolality. Hydrolysis of the chlorine gas for a for a first and/or second period of time can be any time suitable to produce the desired amount of one or more stable free available chlorine species and one or more peroxides.

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides, adding about 0.001% (w/w) to about 0.1% (w/w) of sodium peroxide to the first reaction solution, and introducing chlorine gas at a rate of 240 g/hour to 320 g/hour to the aqueous solution under hydrolysis conditions for about 5 minutes to 75 minutes to produce a second reaction solution comprising one or more stable free available chlorine species and one or more peroxides. The resulting second reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes, the first reaction solution undergoes hydrolysis conditions for about 15 minutes to 75 minutes, about 15 minutes to 60 minutes, or about 30 minutes to 60 minutes, or both the aqueous solution and the first reaction solution undergoes hydrolysis conditions for about 15 minutes to 75 minutes, about 15 minutes to 60 minutes, or about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), or both the amount of sodium peroxide added to the purified water and the first reaction solution is about 0.005% (w/w) to about 0.08% (w/w), about 0.008% (w/w) to about 0.06% (w/w), about 0.01% (w/w) to about 0.04% (w/w).

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides, adding about 0.001% (w/w) to about 0.1% (w/w) of sodium peroxide to the first reaction solution, and introducing chlorine gas at a rate of 240 g/hour to 320 g/hour to the aqueous solution under hydrolysis conditions for about 5 minutes to 75 minutes to produce a second reaction solution comprising one or more stable free available chlorine species and one or more peroxides, and degassing the second reaction solution for 24 hours to 72 hours. The resulting degassed, second reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes, the first reaction solution undergoes hydrolysis conditions for about 15 minutes to 75 minutes, about 15 minutes to 60 minutes, or about 30 minutes to 60 minutes, or both the aqueous solution and the first reaction solution undergoes hydrolysis conditions for about 15 minutes to 75 minutes, about 15 minutes to 60 minutes, or about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), or both the amount of sodium peroxide added to the purified water and the first reaction solution is about 0.005% (w/w) to about 0.08% (w/w), about 0.008% (w/w) to about 0.06% (w/w), about 0.01% (w/w) to about 0.04% (w/w).

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 5 minutes to 75 minutes to produce a first reaction solution comprising one or more stable free available chlorine species and one or more peroxides, adding about 0.001% (w/w) to about 0.1% (w/w) of sodium peroxide to the first reaction solution, and introducing chlorine gas at a rate of 240 g/hour to 320 g/hour to the aqueous solution under hydrolysis conditions for about 30 minutes to 60 minutes to produce a second reaction solution comprising one or more stable free available chlorine species and one or more peroxides, degassing the second reaction solution for 24 hours to 72 hours, and adding about 0.5% to about 1.0% of sodium chloride to the degassed second reaction solution to adjust the osmolality to a range of about 275 mOsm/kg to about 295 mOsm/kg. The resulting osmolarity adjusted, degassed, second reaction solution produces a composition comprising one or more stable free available chlorine species and one or more peroxides that are stably maintained within the composition. In aspects of these embodiments, the aqueous solution undergoes hydrolysis conditions for about 5 minutes to 75 minutes, the first reaction solution undergoes hydrolysis conditions for about 30 minutes to 60 minutes, or both the aqueous solution and the first reaction solution undergoes hydrolysis conditions for about 30 minutes to 60 minutes. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), or both the amount of sodium peroxide added to the purified water and the first reaction solution is about 0.005% (w/w) to about 0.08% (w/w), about 0.008% (w/w) to about 0.06% (w/w), about 0.01% (w/w) to about 0.04% (w/w). In aspects of these embodiments, the amount of sodium chloride added to the composition is about 0.6% to about 0.9%, or about 0.7% to about 0.8%.

In some embodiments, a method of manufacture disclosed herein comprises adding sodium peroxide to purified water contained in a sealed vessel to prepare an aqueous solution, introducing chlorine gas to the aqueous solution under hydrolysis conditions for a first period of time to produce a first reaction solution comprising one or more stable free available chlorine species and one or more peroxides, adding sodium peroxide to the first reaction solution, introducing chlorine gas to the first reaction solution under hydrolysis conditions for a second period of time to produce a second reaction solution comprising one or more stable free available chlorine species and one or more peroxides, adding sodium peroxide to the second reaction solution, introducing chlorine gas to the second reaction solution under hydrolysis conditions for a third period of time to produce a third reaction solution comprising one or more stable free available chlorine species and one or more peroxides. A method of manufacture disclosed herein can further comprise degassing the third reaction solution for a degassing period of time. Optionally, sodium phosphate and/or sodium chloride can be added to an aqueous solution disclosed herein. Optionally, a method of manufacture disclosed herein further comprises adding sodium chloride to the third reaction solution to adjust the osmolality to a physiological compatible osmolality. Hydrolysis of the chlorine gas for a for a first and/or second and/or third period of time can be any time suitable to produce the desired amount of one or more stable free available chlorine species and one or more peroxides.

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 30 minutes to 60 minutes to produce a first reaction solution comprising one or more stable free available chlorine species and one or more peroxides, adding about 0.001% (w/w) to about 0.1% (w/w) of sodium peroxide to the first reaction solution, introducing chlorine gas at a rate of 240 g/hour to 320 g/hour to the aqueous solution under hydrolysis conditions for about 30 minutes to 60 minutes to produce a second reaction solution comprising one or more stable free available chlorine species and one or more peroxides, adding about 0.001% (w/w) to about 0.1% (w/w) of sodium peroxide to the second reaction solution, and introducing chlorine gas at a rate of 240 g/hour to 320 g/hour to the aqueous solution under hydrolysis conditions for about 30 minutes to 60 minutes to produce a third reaction solution comprising one or more stable free available chlorine species and one or more peroxides. The resulting third reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes, the first reaction solution undergoes hydrolysis conditions for about 15 minutes to 75 minutes, about 15 minutes to 60 minutes, or about 30 minutes to 60 minutes, the second reaction solution undergoes hydrolysis conditions for about 5 minutes to 15 minutes, about 15 minutes to 75 minutes, about 15 minutes to 60 minutes, or about 30 minutes to 60 minutes, or any combination thereof. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), the amount of sodium peroxide added to the second reaction solution is about 0.005% (w/w) to about 0.08% (w/w), about 0.008% (w/w) to about 0.06% (w/w), about 0.01% (w/w) to about 0.04% (w/w), or any combination thereof.

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 30 minutes to 60 minutes to produce a first reaction solution comprising one or more stable free available chlorine species and one or more peroxides, adding about 0.001% (w/w) to about 0.1% (w/w) of sodium peroxide to the first reaction solution, introducing chlorine gas at a rate of 240 g/hour to 320 g/hour to the aqueous solution under hydrolysis conditions for about 30 minutes to 60 minutes to produce a second reaction solution comprising one or more stable free available chlorine species and one or more peroxides, adding about 0.001% (w/w) to about 0.1% (w/w) of sodium peroxide to the second reaction solution, introducing chlorine gas at a rate of 240 g/hour to 320 g/hour to the aqueous solution under hydrolysis conditions for about 30 minutes to 60 minutes to produce a third reaction solution comprising one or more stable free available chlorine species and one or more peroxides, and degassing the third reaction solution for 24 hours to 72 hours. The resulting degassed, third reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes, the first reaction solution undergoes hydrolysis conditions for about 15 minutes to 75 minutes, about 15 minutes to 60 minutes, or about 30 minutes to 60 minutes, the second reaction solution undergoes hydrolysis conditions for about 5 minutes to 15 minutes, about 15 minutes to 75 minutes, about 15 minutes to 60 minutes, or about 30 minutes to 60 minutes, or any combination thereof. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), the amount of sodium peroxide added to the second reaction solution is about 0.005% (w/w) to about 0.08% (w/w), about 0.008% (w/w) to about 0.06% (w/w), about 0.01% (w/w) to about 0.04% (w/w), or any combination thereof.

In some embodiments, a method of manufacture disclosed herein comprises adding about 0.02% (w/w) to about 0.24% (w/w) of sodium peroxide to purified water contained in a vessel to prepare an aqueous solution, introducing chlorine gas at a rate of 240 g/hour to 320 g/hour to the aqueous solution under hydrolysis conditions for about 30 minutes to 60 minutes to produce a first reaction solution comprising one or more stable free available chlorine species and one or more peroxides, adding about 0.001% (w/w) to about 0.1% (w/w) of sodium peroxide to the first reaction solution, introducing chlorine gas at a rate of 240 g/hour to 320 g/hour to the aqueous solution under hydrolysis conditions for about 30 minutes to 60 minutes to produce a second reaction solution comprising one or more stable free available chlorine species and one or more peroxides, adding about 0.001% (w/w) to about 0.1% (w/w) of sodium peroxide to the second reaction solution, introducing chlorine gas at a rate of 240 g/hour to 320 g/hour to the aqueous solution under hydrolysis conditions for about 30 minutes to 60 minutes to produce a third reaction solution comprising one or more stable free available chlorine species and one or more peroxides, degassing the third reaction solution for 24 hours to 72 hours, and adding about 0.5% to about 1.0% of sodium chloride to the degassed third reaction solution to adjust the osmolality to a range of about 275 mOsm/kg to about 295 mOsm/kg. The resulting osmolarity adjusted, degassed, third reaction solution produces a composition comprising one or more stable free available chlorine species and one or more peroxides that are stably maintained within the composition. In aspects of these embodiments, the aqueous solution undergoes hydrolysis conditions for about 15 minutes to 75 minutes, about 15 minutes to 60 minutes, or about 30 minutes to 60 minutes, the first reaction solution undergoes hydrolysis conditions for about 15 minutes to 75 minutes, about 15 minutes to 60 minutes, or about 30 minutes to 60 minutes, the second reaction solution undergoes hydrolysis conditions for about 5 minutes to 15 minutes, about 15 minutes to 75 minutes, about 15 minutes to 60 minutes, or about 30 minutes to 60 minutes, or any combination thereof. In aspects of these embodiments, the amount of sodium peroxide added to the purified water is about 0.04% (w/w) to about 0.2% (w/w), about 0.05% (w/w) to about 0.16% (w/w), or about 0.06% (w/w) to about 0.12% (w/w), the amount of sodium peroxide added to the first reaction solution is about 0.005% (w/w) to about 0.08% (w/w), about 0.008% (w/w) to about 0.06% (w/w), about 0.01% (w/w) to about 0.04% (w/w), the amount of sodium peroxide added to the second reaction solution is about 0.005% (w/w) to about 0.08% (w/w), about 0.008% (w/w) to about 0.06% (w/w), about 0.01% (w/w) to about 0.04% (w/w), or any combination thereof. In aspects of these embodiments, the amount of sodium chloride added to the composition is about 0.6% to about 0.9%, or about 0.7% to about 0.8%.

In a method of manufacture disclosed herein the amount of one or more free available chlorine species produced in the first reaction solution, second reaction solution, and third reaction solution can be controlled by the length of the period of time the aqueous solution, the first reaction solution, and the second reaction solution is maintained under hydrolysis conditions. The longer hydrolysis conditions are maintained, the more one or more free available chlorine species is produced in the first reaction solution, second reaction solution, and third reaction solution.

Empirically, given the amount of sodium peroxide used in a method of manufacture disclosed herein an equimolar amount of one or more peroxides and one or more free available chlorine species will be produced. In some embodiments, where a degassing step of the resulting composition is performed, there will be some loss of the one or more peroxides. In some embodiments, where the one or more peroxides are stabilized there can be a higher concentration of one or more peroxides relative to the one or more free available chlorine species. In some embodiments, the concentration of one or more peroxides present is in the first reaction solution, the second reaction solution, and/or the third reaction solution is less than the concentration of the one or more free available chlorine species present in the that reaction solution. In some embodiments, the concentration of one or more peroxides present is in the first reaction solution, the second reaction solution, and/or the third reaction solution is the same as the concentration of the one or more free available chlorine species present in the that reaction solution. In some embodiments, the concentration of one or more peroxides present is in the first reaction solution, the second reaction solution, and/or the third reaction solution is more than the concentration of the one or more free available chlorine species present in the that reaction solution.

In some embodiments, exogenous hydrogen peroxide can be added to a composition manufactured according to a method or use disclosed herein. Such exogenous hydrogen peroxide is not created using a method or use disclosed herein. In some embodiments, the amount of hydrogen peroxide added is an amount that provides a desired beneficial effect to a composition disclosed herein. In some embodiments, when a composition manufactured according to a method or use disclosed herein comprising hydrogen peroxide is formulated to be used in an undiluted state, the amount of hydrogen peroxide used is an amount effective to clean, disinfect, sanitize, and/or sterilize. In some embodiments, when a composition manufactured according to a method or use disclosed herein comprising hydrogen peroxide is formulated to be used in an undiluted state, the amount of hydrogen peroxide used is a microbiostatic effective amount. A microbiostatic effective amount of hydrogen peroxide is an amount that restrains, prohibits, or otherwise prevents microbial population growth without eradicating the microbes. In an aspect of this embodiment, a microbiostatic effective amount of hydrogen peroxide is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising hydrogen peroxide will be diluted, the amount of hydrogen peroxide used in such concentrated formulations can be any amount that does not cause precipitation of components in the composition disclosed herein or otherwise create unwanted effects that make the composition inoperable.

In aspects of this embodiment, a composition disclosed herein has hydrogen peroxide added to an amount of, e.g., 0.5%, 1%, 2%, 3%, 4%, 5%, 6%, 7%, 8%, 9% or 10% by weight of the composition. In other aspects of this embodiment, a composition disclosed herein has hydrogen peroxide added to an amount of, e.g., at least 0.5%, at least 1%, at least 2%, at least 3%, at least 4%, at least 5%, at least 6%, at least 7%, at least 8%, at least 9% or at least 10% by weight of the composition. In yet other aspects of this embodiment, a composition disclosed herein has hydrogen peroxide added to an amount of, e.g., at most 0.5%, at most 1%, at most 2%, at most 3%, at most 4%, at most 5%, at most 6%, at most 7%, at most 8%, at most 9% or at most 10% by weight of the composition. In yet other aspects of this embodiment, a composition disclosed herein has hydrogen peroxide added to an amount of, e.g., about 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% by weight of the composition.

In some embodiments, one or more metallic particles can be added to a composition manufactured according to a method or use disclosed herein. A metallic particle can be composed of a single element, such as, e.g., copper, iron, silver, titanium, or zinc or be a mixed metallic particle composed of various combinations of different elements, such as, e.g., various combinations of two or more of the following: copper, iron, silver, titanium, or zinc. Non-limiting examples of a metallic particle includes a copper particle, an iron particle, a potassium particle, a silver particle, a titanium particle, and a zinc particle. Other non-limiting examples of a metallic particle include a metal acetate particle, a metal chloride particle, a metal nitrate particle, or a metal oxide particle. A metal acetate particle includes, without limitation, copper acetate, iron acetate, e.g., iron (II) acetate and iron (III) acetate, silver acetate, titanium acetate, zinc acetate, or any combination thereof. A metal nitrate particle includes, without limitation, copper nitrate, iron nitrate, e.g., iron (II) nitrate, iron (III) nitrate, silver nitrate, titanium nitrate, zinc nitrate, or any combination thereof. A metal chloride particle includes, without limitation, copper chloride, iron chloride, e.g., iron (II) chloride or iron (III) chloride, silver chloride, titanium chloride, zinc chloride, or any combination thereof. A metal oxide particle includes, without limitation, copper oxide particle, iron oxide particle, e.g., iron (II) oxide particle, iron (III) oxide particle, silver oxide particle, titanium dioxide particle, zinc oxide particle, copper zinc iron oxide particle, or any combination thereof.

A metallic particle includes nanoparticles and microparticles. A nanoparticle has an average diameter of about 1 nm to about 1,000 nm. A microparticle has an average diameter of about 1 μm to about 1,000 μm.

In some embodiments, the amount of metallic particles added is an amount that provides a desired beneficial effect to a composition disclosed herein. In some embodiments, when a composition manufactured according to a method or use disclosed herein comprising metallic particles is formulated to be used in an undiluted state, the amount of metallic particles used is an amount effective to clean, disinfect, sanitize, and/or sterilize. In some embodiments, when a composition manufactured according to a method or use disclosed herein comprising metallic particles is formulated to be used in an undiluted state, the amount of metallic particles used is a microbiostatic effective amount. A microbiostatic effective amount of metallic particles is an amount that restrains, prohibits, or otherwise prevents microbial population growth without eradicating the microbes. In an aspect of this embodiment, a microbiostatic effective amount of metallic particles is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising metallic particles will be diluted, the amount of metallic particles used in such concentrated formulations can be any amount that does not cause precipitation of components in the composition disclosed herein or otherwise create unwanted effects that make the composition inoperable.

In aspects of this embodiment, a composition disclosed herein has metallic particles added to a final concentration of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein has metallic particles added to a final concentration of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein has metallic particles added to a final concentration of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein has metallic particles added to a final concentration of, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein has metallic particles added to a final concentration of, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 300 ppm to about 325 ppm, about 300 ppm to about 350 ppm, about 300 ppm to about 375 ppm, about 300 ppm to about 400 ppm, about 350 ppm to about 375 ppm, about 350 ppm to about 400 ppm, or about 375 ppm to about 400 ppm.

In other aspects of this embodiment, a composition disclosed herein has metallic particles added to a final concentration of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm.

In aspects of this embodiment, a composition disclosed herein has metallic particles added to a final concentration of, e.g., 0.05 mg/L, 0.10 mg/L, 0.15 mg/L, 0.20 mg/L, 0.25 mg/L, 0.30 mg/L, 0.35 mg/L, 0.40 mg/L, 0.45 mg/L, 0.50 mg/L, 0.55 mg/L, 0.60 mg/L, 0.65 mg/L, 0.70 mg/L, 0.75 mg/L, 0.80 mg/L, 0.85 mg/L, 0.90 mg/L, 0.95 mg/L, 1 mg/L, 5 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, 70 mg/L, 75 mg/L, 80 mg/L, 85 mg/L, 90 mg/L, 95 mg/L, 100 mg/L, 125 mg/L, 150 mg/L, 175 mg/L, 200 mg/L, 225 mg/L, 250 mg/L, 275 mg/L, 300 mg/L, 325 mg/L, 350 mg/L, 375 mg/L, 400 mg/L, 425 mg/L, 450 mg/L, 475 mg/L, 500 mg/L, 525 mg/L, 550 mg/L, 575 mg/L, 600 mg/L, 625 mg/L, 650 mg/L, 675 mg/L, 700 mg/L, 725 mg/L, 750 mg/L, 775 mg/L, 800 mg/L, 825 mg/L, 850 mg/L, 875 mg/L, 900 mg/L, 925 mg/L, 950 mg/L, 975 mg/L, 1,000 mg/L, 1,025 mg/L, 1,050 mg/L, 1075 mg/L, 1,100 mg/L, 1,125 mg/L, 1,150 mg/L, 1,175 mg/L, 1,200 mg/L, 1,225 mg/L, 1,250 mg/L, 1,275 mg/L, 1,300 mg/L, 1,325 mg/L, 1,350 mg/L, 1,375 mg/L, 1,400 mg/L, 1,425 mg/L, 1,450 mg/L, 1,475 mg/L, 1,500 mg/L, 1,600 mg/L, 1,700 mg/L, 1,800 mg/L, 1,900 mg/L, or 2,000 mg/L. In other aspects of this embodiment, a composition disclosed herein has metallic particles added to a final concentration of, e.g., at least 0.05 mg/L, at least 0.10 mg/L, at least 0.20 mg/L, at least 0.30 mg/L, at least 0.40 mg/L, at least 0.50 mg/L, at least 0.60 mg/L, at least 0.70 mg/L, at least 0.80 mg/L, at least 0.90 mg/L, at least 1 mg/L, at least 10 mg/L, at least 20 mg/L, at least 30 mg/L, at least 40 mg/L, at least 50 mg/L, at least 60 mg/L, at least 70 mg/L, at least 80 mg/L, at least 90 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 525 mg/L, at least 550 mg/L, at least 575 mg/L, at least 600 mg/L, at least 625 mg/L, at least 650 mg/L, at least 675 mg/L, at least 700 mg/L, at least 725 mg/L, at least 750 mg/L, at least 775 mg/L, at least 800 mg/L, at least 825 mg/L, at least 850 mg/L, at least 875 mg/L, at least 900 mg/L, at least 925 mg/L, at least 950 mg/L, at least 975 mg/L, at least 1,000 mg/L, at least 1,025 mg/L, at least 1,050 mg/L, at least 1075 mg/L, at least 1,100 mg/L, at least 1,125 mg/L, at least 1,150 mg/L, at least 1,175 mg/L, at least 1,200 mg/L, at least 1,225 mg/L, at least 1,250 mg/L, at least 1,275 mg/L, at least 1,300 mg/L, at least 1,325 mg/L, at least 1,350 mg/L, at least 1,375 mg/L, at least 1,400 mg/L, at least 1,425 mg/L, at least 1,450 mg/L, at least 1,475 mg/L, at least 1,500 mg/L, at least 1,600 mg/L, at least 1,700 mg/L, at least 1,800 mg/L, at least 1,900 mg/L, or at least 2,000 mg/L. In yet other aspects of this embodiment, a composition disclosed herein has metallic particles added to a final concentration of, e.g., at most 0.05 mg/L, at most 0.10 mg/L, at most 0.20 mg/L, at most 0.30 mg/L, at most 0.40 mg/L, at most 0.50 mg/L, at most 0.60 mg/L, at most 0.70 mg/L, at most 0.80 mg/L, at most 0.90 mg/L, at most 1 mg/L, at most 10 mg/L, at most 20 mg/L, at most 30 mg/L, at most 40 mg/L, at most 50 mg/L, at most 60 mg/L, at most 70 mg/L, at most 80 mg/L, at most 90 mg/L, at most 100 mg/L, at most 125 mg/L, at most 150 mg/L, at most 175 mg/L, at most 200 mg/L, at most 225 mg/L, at most 250 mg/L, at most 275 mg/L, at most 300 mg/L, at most 325 mg/L, at most 350 mg/L, at most 375 mg/L, at most 400 mg/L, at most 425 mg/L, at most 450 mg/L, at most 475 mg/L, at most 500 mg/L, at most 525 mg/L, at most 550 mg/L, at most 575 mg/L, at most 600 mg/L, at most 625 mg/L, at most 650 mg/L, at most 675 mg/L, at most 700 mg/L, at most 725 mg/L, at most 750 mg/L, at most 775 mg/L, at most 800 mg/L, at most 825 mg/L, at most 850 mg/L, at most 875 mg/L, at most 900 mg/L, at most 925 mg/L, at most 950 mg/L, at most 975 mg/L, at most 1,000 mg/L, at most 1,025 mg/L, at most 1,050 mg/L, at most 1075 mg/L, at most 1,100 mg/L, at most 1,125 mg/L, at most 1,150 mg/L, at most 1,175 mg/L, at most 1,200 mg/L, at most 1,225 mg/L, at most 1,250 mg/L, at most 1,275 mg/L, at most 1,300 mg/L, at most 1,325 mg/L, at most 1,350 mg/L, at most 1,375 mg/L, at most 1,400 mg/L, at most 1,425 mg/L, at most 1,450 mg/L, at most 1,475 mg/L, at most 1,500 mg/L, at most 1,600 mg/L, at most 1,700 mg/L, at most 1,800 mg/L, at most 1,900 mg/L, or at most 2,000 mg/L.

In still other aspects of this embodiment, a composition disclosed herein has metallic particles added to a final concentration of, e.g., about 0.5 mg/L to about 20 mg/L, about 0.5 mg/L to about 25 mg/L, about 0.5 mg/L to about 30 mg/L, about 0.5 mg/L to about 35 mg/L, about 0.5 mg/L to about 40 mg/L, about 0.5 mg/L to about 45 mg/L, about 0.5 mg/L to about 50 mg/L, about 0.5 mg/L to about 55 mg/L, about 0.5 mg/L to about 60 mg/L, about 0.5 mg/L to about 65 mg/L, about 0.5 mg/L to about 70 mg/L, about 0.5 mg/L to about 75 mg/L, about 0.5 mg/L to about 80 mg/L, about 0.5 mg/L to about 85 mg/L, about 0.5 mg/L to about 90 mg/L, about 0.5 mg/L to about 95 mg/L, about 0.5 mg/L to about 100 mg/L, about 0.75 mg/L to about 20 mg/L, about 0.75 mg/L to about 25 mg/L, about 0.75 mg/L to about 30 mg/L, about 0.75 mg/L to about 35 mg/L, about 0.75 mg/L to about 40 mg/L, about 0.75 mg/L to about 45 mg/L, about 0.75 mg/L to about 50 mg/L, about 0.75 mg/L to about 55 mg/L, about 0.75 mg/L to about 60 mg/L, about 0.75 mg/L to about 65 mg/L, about 0.75 mg/L to about 70 mg/L, about 0.75 mg/L to about 75 mg/L, about 0.75 mg/L to about 80 mg/L, about 0.75 mg/L to about 85 mg/L, about 0.75 mg/L to about 90 mg/L, about 0.75 mg/L to about 95 mg/L, about 0.75 mg/L to about 100 mg/L, about 1 mg/L to about 20 mg/L, about 1 mg/L to about 25 mg/L, about 1 mg/L to about 30 mg/L, about 1 mg/L to about 35 mg/L, about 1 mg/L to about 40 mg/L, about 1 mg/L to about 45 mg/L, about 1 mg/L to about 50 mg/L, about 1 mg/L to about 55 mg/L, about 1 mg/L to about 60 mg/L, about 1 mg/L to about 65 mg/L, about 1 mg/L to about 70 mg/L, about 1 mg/L to about 75 mg/L, about 1 mg/L to about 80 mg/L, about 1 mg/L to about 85 mg/L, about 1 mg/L to about 90 mg/L, about 1 mg/L to about 95 mg/L, about 1 mg/L to about 100 mg/L, about 5 mg/L to about 20 mg/L, about 5 mg/L to about 25 mg/L, about 5 mg/L to about 30 mg/L, about 5 mg/L to about 35 mg/L, about 5 mg/L to about 40 mg/L, about 5 mg/L to about 45 mg/L, about 5 mg/L to about 50 mg/L, about 5 mg/L to about 55 mg/L, about 5 mg/L to about 60 mg/L, about 5 mg/L to about 65 mg/L, about 5 mg/L to about 70 mg/L, about 5 mg/L to about 75 mg/L, about 5 mg/L to about 80 mg/L, about 5 mg/L to about 85 mg/L, about 5 mg/L to about 90 mg/L, about 5 mg/L to about 95 mg/L, about 5 mg/L to about 100 mg/L, about 10 mg/L to about 20 mg/L, about 10 mg/L to about 25 mg/L, about 10 mg/L to about 30 mg/L, about 10 mg/L to about 35 mg/L, about 10 mg/L to about 40 mg/L, about 10 mg/L to about 45 mg/L, about 10 mg/L to about 50 mg/L, about 10 mg/L to about 55 mg/L, about 10 mg/L to about 60 mg/L, about 10 mg/L to about 65 mg/L, about 10 mg/L to about 70 mg/L, about 10 mg/L to about 75 mg/L, about 10 mg/L to about 80 mg/L, about 10 mg/L to about 85 mg/L, about 10 mg/L to about 90 mg/L, about 10 mg/L to about 95 mg/L, or about 10 mg/L to about 100 mg/L.

In other aspects of this embodiment, a composition disclosed herein has metallic particles added to a final concentration of, e.g., about 1 mg/L to about 25 mg/L, about 1 mg/L to about 50 mg/L, about 1 mg/L to about 75 mg/L, about 1 mg/L to about 100 mg/L, about 1 mg/L to about 125 mg/L, about 1 mg/L to about 150 mg/L, about 1 mg/L to about 175 mg/L, about 1 mg/L to about 200 mg/L, about 1 mg/L to about 225 mg/L, about 1 mg/L to about 250 mg/L, about 1 mg/L to about 275 mg/L, about 1 mg/L to about 300 mg/L, about 1 mg/L to about 325 mg/L, about 1 mg/L to about 350 mg/L, about 1 mg/L to about 375 mg/L, about 1 mg/L to about 400 mg/L, about 10 mg/L to about 25 mg/L, about 10 mg/L to about 50 mg/L, about 10 mg/L to about 75 mg/L, about 10 mg/L to about 100 mg/L, about 10 mg/L to about 125 mg/L, about 10 mg/L to about 150 mg/L, about 10 mg/L to about 175 mg/L, about 10 mg/L to about 200 mg/L, about 10 mg/L to about 225 mg/L, about 10 mg/L to about 250 mg/L, about 10 mg/L to about 275 mg/L, about 10 mg/L to about 300 mg/L, about 10 mg/L to about 325 mg/L, about 10 mg/L to about 350 mg/L, about 10 mg/L to about 375 mg/L, about 10 mg/L to about 400 mg/L, about 25 mg/L to about 50 mg/L, about 25 mg/L to about 75 mg/L, about 25 mg/L to about 100 mg/L, about 25 mg/L to about 125 mg/L, about 25 mg/L to about 150 mg/L, about 25 mg/L to about 175 mg/L, about 25 mg/L to about 200 mg/L, about 25 mg/L to about 225 mg/L, about 25 mg/L to about 250 mg/L, about 25 mg/L to about 275 mg/L, about 25 mg/L to about 300 mg/L, about 25 mg/L to about 325 mg/L, about 25 mg/L to about 350 mg/L, about 25 mg/L to about 375 mg/L, about 25 mg/L to about 400 mg/L, about 50 mg/L to about 75 mg/L, about 50 mg/L to about 100 mg/L, about 50 mg/L to about 125 mg/L, about 50 mg/L to about 150 mg/L, about 50 mg/L to about 175 mg/L, about 50 mg/L to about 200 mg/L, about 50 mg/L to about 225 mg/L, about 50 mg/L to about 250 mg/L, about 50 mg/L to about 275 mg/L, about 50 mg/L to about 300 mg/L, about 50 mg/L to about 325 mg/L, about 50 mg/L to about 350 mg/L, about 50 mg/L to about 375 mg/L, about 50 mg/L to about 400 mg/L, about 75 mg/L to about 100 mg/L, about 75 mg/L to about 125 mg/L, about 75 mg/L to about 150 mg/L, about 75 mg/L to about 175 mg/L, about 75 mg/L to about 200 mg/L, about 75 mg/L to about 225 mg/L, about 75 mg/L to about 250 mg/L, about 75 mg/L to about 275 mg/L, about 75 mg/L to about 300 mg/L, about 75 mg/L to about 325 mg/L, about 75 mg/L to about 350 mg/L, about 75 mg/L to about 375 mg/L, about 75 mg/L to about 400 mg/L, about 100 mg/L to about 125 mg/L, about 100 mg/L to about 150 mg/L, about 100 mg/L to about 175 mg/L, about 100 mg/L to about 200 mg/L, about 100 mg/L to about 225 mg/L, about 100 mg/L to about 250 mg/L, about 100 mg/L to about 275 mg/L, about 100 mg/L to about 300 mg/L, about 100 mg/L to about 325 mg/L, about 100 mg/L to about 350 mg/L, about 100 mg/L to about 375 mg/L, about 100 mg/L to about 400 mg/L, about 150 mg/L to about 175 mg/L, about 150 mg/L to about 200 mg/L, about 150 mg/L to about 225 mg/L, about 150 mg/L to about 250 mg/L, about 150 mg/L to about 275 mg/L, about 150 mg/L to about 300 mg/L, about 150 mg/L to about 325 mg/L, about 150 mg/L to about 350 mg/L, about 150 mg/L to about 375 mg/L, about 150 mg/L to about 400 mg/L, about 200 mg/L to about 225 mg/L, about 200 mg/L to about 250 mg/L, about 200 mg/L to about 275 mg/L, about 200 mg/L to about 300 mg/L, about 200 mg/L to about 325 mg/L, about 200 mg/L to about 350 mg/L, about 200 mg/L to about 375 mg/L, about 200 mg/L to about 400 mg/L, about 250 mg/L to about 275 mg/L, about 250 mg/L to about 300 mg/L, about 250 mg/L to about 325 mg/L, about 250 mg/L to about 350 mg/L, about 250 mg/L to about 375 mg/L, about 250 mg/L to about 400 mg/L, about 300 mg/L to about 325 mg/L, about 300 mg/L to about 350 mg/L, about 300 mg/L to about 375 mg/L, about 300 mg/L to about 400 mg/L, about 350 mg/L to about 375 mg/L, about 350 mg/L to about 400 mg/L, or about 375 mg/L to about 400 mg/L.

In other aspects of this embodiment, a composition disclosed herein has metallic particles added to a final concentration of, e.g., about 400 mg/L to about 500 mg/L, about 400 mg/L to about 600 mg/L, about 400 mg/L to about 700 mg/L, about 400 mg/L to about 800 mg/L, about 400 mg/L to about 900 mg/L, about 400 mg/L to about 1,000 mg/L, about 400 mg/L to about 1,100 mg/L, about 400 mg/L to about 1,200 mg/L, about 400 mg/L to about 1,300 mg/L, about 400 mg/L to about 1,400 mg/L, about 400 mg/L to about 1,500 mg/L, about 400 mg/L to about 1,600 mg/L, about 400 mg/L to about 1,700 mg/L, about 400 mg/L to about 1,800 mg/L, about 400 mg/L to about 1,900 mg/L, about 400 mg/L to about 2,000 mg/L, about 500 mg/L to about 600 mg/L, about 500 mg/L to about 700 mg/L, about 500 mg/L to about 800 mg/L, about 500 mg/L to about 900 mg/L, about 500 mg/L to about 1,000 mg/L, about 500 mg/L to about 1,100 mg/L, about 500 mg/L to about 1,200 mg/L, about 500 mg/L to about 1,300 mg/L, about 500 mg/L to about 1,400 mg/L, about 500 mg/L to about 1,500 mg/L, about 500 mg/L to about 1,600 mg/L, about 500 mg/L to about 1,700 mg/L, about 500 mg/L to about 1,800 mg/L, about 500 mg/L to about 1,900 mg/L, about 500 mg/L to about 2,000 mg/L, about 600 mg/L to about 700 mg/L, about 600 mg/L to about 800 mg/L, about 600 mg/L to about 900 mg/L, about 600 mg/L to about 1,000 mg/L, about 600 mg/L to about 1,100 mg/L, about 600 mg/L to about 1,200 mg/L, about 600 mg/L to about 1,300 mg/L, about 600 mg/L to about 1,400 mg/L, about 600 mg/L to about 1,500 mg/L, about 600 mg/L to about 1,600 mg/L, about 600 mg/L to about 1,700 mg/L, about 600 mg/L to about 1,800 mg/L, about 600 mg/L to about 1,900 mg/L, about 600 mg/L to about 2,000 mg/L, about 700 mg/L to about 800 mg/L, about 700 mg/L to about 900 mg/L, about 700 mg/L to about 1,000 mg/L, about 700 mg/L to about 1,100 mg/L, about 700 mg/L to about 1,200 mg/L, about 700 mg/L to about 1,300 mg/L, about 700 mg/L to about 1,400 mg/L, about 700 mg/L to about 1,500 mg/L, about 700 mg/L to about 1,600 mg/L, about 700 mg/L to about 1,700 mg/L, about 700 mg/L to about 1,800 mg/L, about 700 mg/L to about 1,900 mg/L, about 700 mg/L to about 2,000 mg/L, about 800 mg/L to about 900 mg/L, about 800 mg/L to about 1,000 mg/L, about 800 mg/L to about 1,100 mg/L, about 800 mg/L to about 1,200 mg/L, about 800 mg/L to about 1,300 mg/L, about 800 mg/L to about 1,400 mg/L, about 800 mg/L to about 1,500 mg/L, about 800 mg/L to about 1,600 mg/L, about 800 mg/L to about 1,700 mg/L, about 800 mg/L to about 1,800 mg/L, about 800 mg/L to about 1,900 mg/L, about 800 mg/L to about 2,000 mg/L, about 900 mg/L to about 1,000 mg/L, about 900 mg/L to about 1,100 mg/L, about 900 mg/L to about 1,200 mg/L, about 900 mg/L to about 1,300 mg/L, about 900 mg/L to about 1,400 mg/L, about 900 mg/L to about 1,500 mg/L, about 900 mg/L to about 1,600 mg/L, about 900 mg/L to about 1,700 mg/L, about 900 mg/L to about 1,800 mg/L, about 900 mg/L to about 1,900 mg/L, about 900 mg/L to about 2,000 mg/L, about 1,000 mg/L to about 1,100 mg/L, about 1,000 mg/L to about 1,200 mg/L, about 1,000 mg/L to about 1,300 mg/L, about 1,000 mg/L to about 1,400 mg/L, about 1,000 mg/L to about 1,500 mg/L, about 1,000 mg/L to about 1,600 mg/L, about 1,000 mg/L to about 1,700 mg/L, about 1,000 mg/L to about 1,800 mg/L, about 1,000 mg/L to about 1,900 mg/L, about 1,000 mg/L to about 2,000 mg/L, about 1,100 mg/L to about 1,200 mg/L, about 1,100 mg/L to about 1,300 mg/L, about 1,100 mg/L to about 1,400 mg/L, about 1,100 mg/L to about 1,500 mg/L, about 1,100 mg/L to about 1,600 mg/L, about 1,100 mg/L to about 1,700 mg/L, about 1,100 mg/L to about 1,800 mg/L, about 1,100 mg/L to about 1,900 mg/L, about 1,100 mg/L to about 2,000 mg/L, about 1,200 mg/L to about 1,300 mg/L, about 1,200 mg/L to about 1,400 mg/L, about 1,200 mg/L to about 1,500 mg/L, about 1,200 mg/L to about 1,600 mg/L, about 1,200 mg/L to about 1,700 mg/L, about 1,200 mg/L to about 1,800 mg/L, about 1,200 mg/L to about 1,900 mg/L, about 1,200 mg/L to about 2,000 mg/L, about 1,300 mg/L to about 1,400 mg/L, about 1,300 mg/L to about 1,500 mg/L, about 1,300 mg/L to about 1,600 mg/L, about 1,300 mg/L to about 1,700 mg/L, about 1,300 mg/L to about 1,800 mg/L, about 1,300 mg/L to about 1,900 mg/L, about 1,300 mg/L to about 2,000 mg/L, about 1,400 mg/L to about 1,500 mg/L, about 1,400 mg/L to about 1,600 mg/L, about 1,400 mg/L to about 1,700 mg/L, about 1,400 mg/L to about 1,800 mg/L, about 1,400 mg/L to about 1,900 mg/L, about 1,400 mg/L to about 2,000 mg/L, about 1,500 mg/L to about 1,600 mg/L, about 1,500 mg/L to about 1,700 mg/L, about 1,500 mg/L to about 1,800 mg/L, about 1,500 mg/L to about 1,900 mg/L, about 1,500 mg/L to about 2,000 mg/L, about 1,600 mg/L to about 1,700 mg/L, about 1,600 mg/L to about 1,800 mg/L, about 1,600 mg/L to about 1,900 mg/L, about 1,600 mg/L to about 2,000 mg/L, about 1,700 mg/L to about 1,800 mg/L, about 1,700 mg/L to about 1,900 mg/L, about 1,700 mg/L to about 2,000 mg/L, about 1,800 mg/L to about 1,900 mg/L, about 1,800 mg/L to about 2,000 mg/L, or about 1,900 mg/L to about 2,000 mg/L.

In aspects of this embodiment, a composition disclosed herein has metallic particles added to an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein has metallic particles added to an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein has metallic particles added to an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein has metallic particles added to an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50% by weight of the composition.

In an embodiment, metallic particles can be of any size that provides a desired beneficial effect to a composition disclosed herein. In aspects of this embodiment, metallic particles disclosed herein have a mean diameter of, e.g., about 10 nm, about 20 nm, about 30 nm, about 40 nm, about 50 nm, about 60 nm, about 70 nm, about 80 nm, about 90 nm, about 100 nm. In other aspects of this embodiment, metallic particles disclosed herein have a mean diameter of, e.g., at least 10 nm, at least 20 nm, at least 30 nm, at least 40 nm, at least 50 nm, at least 60 nm, at least 70 nm, at least 80 nm, at least 90 nm, at least 100 nm. In yet other aspects of this embodiment, metallic particles disclosed herein have a mean diameter of, e.g., at most 10 nm, at most 20 nm, at most 30 nm, at most 40 nm, at most 50 nm, at most 60 nm, at most 70 nm, at most 80 nm, at most 90 nm, at most 100 nm. In yet other aspects of this embodiment, metallic particles disclosed herein have a mean diameter of, e.g., about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm, about 50 nm to about 80 nm, about 50 nm to about 90 nm, about 50 nm to about 100 nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm, or about 90 nm to about 100 nm.

In an embodiment, a composition disclosed herein does not comprise metallic particles.

Aspects of the present specification disclose a composition comprising added one or more metal salts to compositions comprising free available chlorine, or a composition comprising hydrogen peroxide and a stable free available chlorine. A metal salt disclosed herein may include one of more of sodium chloride, potassium chloride, calcium oxide, calcium chloride, sodium phosphate or any combination thereof.

In some embodiments, one or more metal salts can be added to a composition manufactured according to a method or use disclosed herein. A metal salt disclosed herein can be an alkali metal salt, an alkaline earth metal salt, a base metal salt, or a transition metal salt. An alkali metal salt includes a lithium salt, a sodium salt, a potassium salt, a rubidium salt, a cesium salt, and a francium salt. An alkaline earth metal salt includes a beryllium salt, a magnesium salt, a calcium salt, a strontium salt, a barium salt, and a radium salt. A basic metal salt includes an aluminum salt, a gallium salt, an indium salt, a tin salt, a thallium salt, a lead salt, and a bismuth salt. A transition metal salt includes a scandium salt, a titanium salt, a vanadium salt, a chromium salt, a manganese salt, an iron salt, a cobalt salt, a palladium salt, a silver salt, m a nickel salt, a copper salt, a zinc salt, a yttrium salt, Non-limiting examples of a metal salt includes a calcium salt, a copper salt, iron salt, a potassium salt, silver salt, titanium salt, and zinc salt.

Other non-limiting examples of a metal salt includes a metal acetate salt, a metal chloride salt, a metal nitrate salt, a metal oxide salt, and a metal sulfate salt. A metal acetate salt includes, without limitation, calcium acetate, copper acetate, iron acetate, e.g., iron (II) acetate and iron (III) acetate, potassium acetate, silver acetate, titanium acetate, and zinc acetate. A metal chloride salt includes, without limitation, calcium chloride salt, copper chloride, iron chloride, e.g., iron (II) chloride or iron (III) chloride, potassium chloride, silver chloride, titanium chloride, and zinc chloride. A metal nitrate salt includes, without limitation, calcium nitrate salt, copper nitrate, iron nitrate, e.g., iron (II) nitrate, iron (III) nitrate, potassium nitrate, silver nitrate, titanium nitrate, and zinc nitrate. A metal oxide salt includes, without limitation, calcium oxide salt, copper oxide salt, iron oxide salt, e.g., iron (II) oxide salt, iron (III) oxide salt, potassium oxide salt, silver oxide salt, titanium oxide salt, and zinc oxide salt. A metal sulfate salt includes, without limitation, calcium sulfate, copper sulfate, iron sulfate, e.g., iron (II) sulfate, iron (III) sulfate, potassium sulfate, silver sulfate, titanium sulfate, and zinc sulfate.

In some embodiments, a composition may include calcium oxide obtained from an organic source like shells from oceanic organisms. In some embodiment, a composition may include calcium oxide obtained from an inorganic source like by a synthetic chemical process.

In some embodiments, the amount of metal salts added is an amount that provides a desired beneficial effect to a composition disclosed herein. In some embodiments, when a composition manufactured according to a method or use disclosed herein comprising metal salts is formulated to be used in an undiluted state, the amount of metal salts used is an amount effective to clean, disinfect, sanitize, and/or sterilize. In some embodiments, when a composition manufactured according to a method or use disclosed herein comprising metal salts is formulated to be used in an undiluted state, the amount of metal salts used is a microbiostatic effective amount. A microbiostatic effective amount of metal salts is an amount that restrains, prohibits, or otherwise prevents microbial population growth without eradicating the microbes. In an aspect of this embodiment, a microbiostatic effective amount of metal salts is an amount that restrains, prohibits, or otherwise prevents microbial population growth without eradicating the microbes to a degree that achieves disinfection, sanitization, or sterilization. Alternatively, when a composition manufactured according to a method or use disclosed herein comprising metal salts will be diluted, the amount of metal salts used in such concentrated formulations can be any amount that does not cause precipitation of components in the composition disclosed herein or otherwise create unwanted effects that make the composition inoperable.

In aspects of this embodiment, a composition disclosed herein has one or more metal salts each added to a final concentration of, e.g., 0.01 ppm, 0.025 ppm, 0.05 ppm, 0.075 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, 2,000 ppm, 3,000 ppm, 4,000 ppm, 5,000 ppm, 6,000 ppm, 7,000 ppm, 8,000 ppm, 9,000 ppm, 10,000 ppm. In other aspects of this embodiment, a composition disclosed herein has one or more metal salts each added to a final concentration of, e.g., at least 0.01 ppm, at least 0.025 ppm, at least 0.05 ppm, at least 0.075 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein has one or more metal salts each added to a final concentration of, e.g., at most 0.01 ppm, at most 0.025 ppm, at most 0.05 ppm, at most 0.075 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm, or at most 3,000 ppm or at most 4,000 ppm or at most 5,000 ppm or at most 6,000 ppm or at most 7,000 ppm or at most 8,000 ppm or at most 9,000 ppm or at most 10,000 ppm.

In still other aspects of this embodiment, a composition disclosed herein has one or more metal salts each added to a final concentration of, e.g., about 0.01 ppm to about 0.025 ppm, about 0.01 ppm to about 0.05 ppm, about 0.01 ppm to about 0.075 ppm, about 0.01 ppm to about 0.1 ppm, about 0.01 ppm to about 0.25 ppm, about 0.01 ppm to about 0.5 ppm, about 0.01 ppm to about 0.75 ppm, about 0.01 ppm to about 1 ppm, about 0.01 ppm to about 2 ppm, about 0.01 ppm to about 3 ppm, about 0.01 ppm to about 4 ppm, about 0.01 ppm to about 5 ppm, about 0.025 ppm to about 0.075 ppm, about 0.025 ppm to about 0.1 ppm, about 0.025 ppm to about 0.25 ppm, about 0.025 ppm to about 0.5 ppm, about 0.025 ppm to about 0.75 ppm, about 0.025 ppm to about 1 ppm, about 0.025 ppm to about 2 ppm, about 0.025 ppm to about 3 ppm, about 0.025 ppm to about 4 ppm, about 0.025 ppm to about 5 ppm, about 0.05 ppm to about 0.075 ppm, about 0.05 ppm to about 0.1 ppm, about 0.05 ppm to about 0.25 ppm, about 0.05 ppm to about 0.5 ppm, about 0.05 ppm to about 0.75 ppm, about 0.05 ppm to about 1 ppm, about 0.05 ppm to about 2 ppm, about 0.05 ppm to about 3 ppm, about 0.05 ppm to about 4 ppm, about 0.05 ppm to about 5 ppm, about 0.075 ppm to about 0.1 ppm, about 0.075 ppm to about 0.25 ppm, about 0.075 ppm to about 0.5 ppm, about 0.075 ppm to about 0.75 ppm, about 0.075 ppm to about 1 ppm, about 0.075 ppm to about 2 ppm, about 0.075 ppm to about 3 ppm, about 0.075 ppm to about 4 ppm, about 0.075 ppm to about 5 ppm, about 0.1 ppm to about 0.25 ppm, about 0.1 ppm to about 0.5 ppm, about 0.1 ppm to about 0.75 ppm, about 0.1 ppm to about 1 ppm, about 0.1 ppm to about 2 ppm, about 0.1 ppm to about 3 ppm, about 0.1 ppm to about 4 ppm, or about 0.1 ppm to about 5 ppm.

In still other aspects of this embodiment, a composition disclosed herein has one or more metal salts each added to a final concentration of, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein has one or more metal salts each added to a final concentration of, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 5 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In other aspects of this embodiment, a composition disclosed herein has one or more metal salts each added to a final concentration of, e.g., about 400 ppm to about 500 ppm, about 400 ppm to about 600 ppm, about 400 ppm to about 700 ppm, about 400 ppm to about 800 ppm, about 400 ppm to about 900 ppm, about 400 ppm to about 1,000 ppm, about 400 ppm to about 1,100 ppm, about 400 ppm to about 1,200 ppm, about 400 ppm to about 1,300 ppm, about 400 ppm to about 1,400 ppm, about 400 ppm to about 1,500 ppm, about 400 ppm to about 1,600 ppm, about 400 ppm to about 1,700 ppm, about 400 ppm to about 1,800 ppm, about 400 ppm to about 1,900 ppm, about 400 ppm to about 2,000 ppm, about 500 ppm to about 600 ppm, about 500 ppm to about 700 ppm, about 500 ppm to about 800 ppm, about 500 ppm to about 900 ppm, about 500 ppm to about 1,000 ppm, about 500 ppm to about 1,100 ppm, about 500 ppm to about 1,200 ppm, about 500 ppm to about 1,300 ppm, about 500 ppm to about 1,400 ppm, about 500 ppm to about 1,500 ppm, about 500 ppm to about 1,600 ppm, about 500 ppm to about 1,700 ppm, about 500 ppm to about 1,800 ppm, about 500 ppm to about 1,900 ppm, about 500 ppm to about 2,000 ppm, about 600 ppm to about 700 ppm, about 600 ppm to about 800 ppm, about 600 ppm to about 900 ppm, about 600 ppm to about 1,000 ppm, about 600 ppm to about 1,100 ppm, about 600 ppm to about 1,200 ppm, about 600 ppm to about 1,300 ppm, about 600 ppm to about 1,400 ppm, about 600 ppm to about 1,500 ppm, about 600 ppm to about 1,600 ppm, about 600 ppm to about 1,700 ppm, about 600 ppm to about 1,800 ppm, about 600 ppm to about 1,900 ppm, about 600 ppm to about 2,000 ppm, about 700 ppm to about 800 ppm, about 700 ppm to about 900 ppm, about 700 ppm to about 1,000 ppm, about 700 ppm to about 1,100 ppm, about 700 ppm to about 1,200 ppm, about 700 ppm to about 1,300 ppm, about 700 ppm to about 1,400 ppm, about 700 ppm to about 1,500 ppm, about 700 ppm to about 1,600 ppm, about 700 ppm to about 1,700 ppm, about 700 ppm to about 1,800 ppm, about 700 ppm to about 1,900 ppm, about 700 ppm to about 2,000 ppm, about 800 ppm to about 900 ppm, about 800 ppm to about 1,000 ppm, about 800 ppm to about 1,100 ppm, about 800 ppm to about 1,200 ppm, about 800 ppm to about 1,300 ppm, about 800 ppm to about 1,400 ppm, about 800 ppm to about 1,500 ppm, about 800 ppm to about 1,600 ppm, about 800 ppm to about 1,700 ppm, about 800 ppm to about 1,800 ppm, about 800 ppm to about 1,900 ppm, about 800 ppm to about 2,000 ppm, about 900 ppm to about 1,000 ppm, about 900 ppm to about 1,100 ppm, about 900 ppm to about 1,200 ppm, about 900 ppm to about 1,300 ppm, about 900 ppm to about 1,400 ppm, about 900 ppm to about 1,500 ppm, about 900 ppm to about 1,600 ppm, about 900 ppm to about 1,700 ppm, about 900 ppm to about 1,800 ppm, about 900 ppm to about 1,900 ppm, about 900 ppm to about 2,000 ppm, about 1,000 ppm to about 1,100 ppm, about 1,000 ppm to about 1,200 ppm, about 1,000 ppm to about 1,300 ppm, about 1,000 ppm to about 1,400 ppm, about 1,000 ppm to about 1,500 ppm, about 1,000 ppm to about 1,600 ppm, about 1,000 ppm to about 1,700 ppm, about 1,000 ppm to about 1,800 ppm, about 1,000 ppm to about 1,900 ppm, about 1,000 ppm to about 2,000 ppm, about 1,100 ppm to about 1,200 ppm, about 1,100 ppm to about 1,300 ppm, about 1,100 ppm to about 1,400 ppm, about 1,100 ppm to about 1,500 ppm, about 1,100 ppm to about 1,600 ppm, about 1,100 ppm to about 1,700 ppm, about 1,100 ppm to about 1,800 ppm, about 1,100 ppm to about 1,900 ppm, about 1,100 ppm to about 2,000 ppm, about 1,200 ppm to about 1,300 ppm, about 1,200 ppm to about 1,400 ppm, about 1,200 ppm to about 1,500 ppm, about 1,200 ppm to about 1,600 ppm, about 1,200 ppm to about 1,700 ppm, about 1,200 ppm to about 1,800 ppm, about 1,200 ppm to about 1,900 ppm, about 1,200 ppm to about 2,000 ppm, about 1,300 ppm to about 1,400 ppm, about 1,300 ppm to about 1,500 ppm, about 1,300 ppm to about 1,600 ppm, about 1,300 ppm to about 1,700 ppm, about 1,300 ppm to about 1,800 ppm, about 1,300 ppm to about 1,900 ppm, about 1,300 ppm to about 2,000 ppm, about 1,400 ppm to about 1,500 ppm, about 1,400 ppm to about 1,600 ppm, about 1,400 ppm to about 1,700 ppm, about 1,400 ppm to about 1,800 ppm, about 1,400 ppm to about 1,900 ppm, about 1,400 ppm to about 2,000 ppm, about 1,500 ppm to about 1,600 ppm, about 1,500 ppm to about 1,700 ppm, about 1,500 ppm to about 1,800 ppm, about 1,500 ppm to about 1,900 ppm, about 1,500 ppm to about 2,000 ppm, about 1,600 ppm to about 1,700 ppm, about 1,600 ppm to about 1,800 ppm, about 1,600 ppm to about 1,900 ppm, about 1,600 ppm to about 2,000 ppm, about 1,700 ppm to about 1,800 ppm, about 1,700 ppm to about 1,900 ppm, about 1,700 ppm to about 2,000 ppm, about 1,800 ppm to about 1,900 ppm, about 1,800 ppm to about 2,000 ppm, or about 1,900 ppm to about 2,000 ppm, or about 2,000 ppm to about 3,000 ppm, or about 3,000 ppm to about 4,000 ppm, or about 4,000 ppm to about 5,000 ppm, or about 5,000 ppm to about 6,000 ppm, or about 6,000 ppm to about 7,000 ppm, or about 7,000 ppm to about 8,000 ppm, or about 8,000 ppm to about 9,000 ppm, or about 9,000 ppm to about 10,000 ppm.

In aspects of this embodiment, a composition disclosed herein has one or more metal salts each added to an amount of, e.g., about 0.001%, about 0.005%, about 0.01%, about 0.05%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, or about 0.5% by weight of the composition. In other aspects of this embodiment, a composition disclosed herein has one or more metal salts each added to an amount of, e.g., at least 0.001%, at least 0.005%, at least 0.01%, at least 0.05%, at least 0.10%, at least 0.11%, at least 0.12%, at least 0.13%, at least 0.14%, at least 0.15%, at least 0.16%, at least 0.17%, at least 0.18%, at least 0.19%, at least 0.2%, at least 0.25%, at least 0.3%, at least 0.35%, at least 0.4%, at least 0.45%, or at least 0.5% by weight of the composition. In yet other aspects of this embodiment, a composition disclosed herein has one or more metal salts each added to an amount of, e.g., at most 0.001%, at most 0.005%, at most 0.01%, at most 0.05%, at most 0.10%, at most 0.11%, at most 0.12%, at most 0.13%, at most 0.14%, at most 0.15%, at most 0.16%, at most 0.17%, at most 0.18%, at most 0.19%, at most 0.2%, at most 0.25%, at most 0.3%, at most 0.35%, at most 0.4%, at most 0.45%, or at most 0.5% by weight of the composition. In still other aspects of this embodiment, a composition disclosed herein has one or more metal salts each added to an amount of, e.g., about 0.0010% to about 0.005%, about 0.0010% to about 0.010%, about 0.0010% to about 0.050%, about 0.0010% to about 0.10%, about 0.0010% to about 0.15%, about 0.0010% to about 0.20%, about 0.0010% to about 0.25%, about 0.0010% to about 0.30%, about 0.0010% to about 0.35%, about 0.0010% to about 0.40%, about 0.0010% to about 0.45%, about 0.0010% to about 0.50%, about 0.010% to about 0.050%, about 0.010% to about 0.10%, about 0.010% to about 0.15%, about 0.010% to about 0.20%, about 0.010% to about 0.25%, about 0.010% to about 0.30%, about 0.010% to about 0.35%, about 0.010% to about 0.40%, about 0.010% to about 0.45%, about 0.010% to about 0.50%, about 0.10% to about 0.15%, about 0.10% to about 0.20%, about 0.10% to about 0.25%, about 0.10% to about 0.30%, about 0.10% to about 0.35%, about 0.10% to about 0.40%, about 0.10% to about 0.45%, about 0.10% to about 0.50%, about 0.20% to about 0.25%, about 0.20% to about 0.30%, about 0.20% to about 0.35%, about 0.20% to about 0.40%, about 0.20% to about 0.45%, about 0.20% to about 0.50%, about 0.30% to about 0.35%, about 0.30% to about 0.40%, about 0.30% to about 0.45%, about 0.30% to about 0.50%, about 0.40% to about 0.45%, about 0.40% to about 0.50%, or about 0.40% to about 0.50%, or about 0.50% to about 0.60%, or about 0.60% to about 0.70%, or about 0.70% to about 0.80% or about 0.80% to about 0.90% or about 0.90% to about 1.0% by weight of the composition.

In aspects of this embodiment, a composition disclosed herein has one or more metal salts each added to an amount of, e.g., about 0.5%, about 0.55%, about 0.6%, about 0.65%, about 0.7%, about 0.75%, about 0.8%, about 0.85%, about 0.9%, about 0.95%, about 1.0% by weight of the composition. In other aspects of this embodiment, a composition disclosed herein has one or more metal salts each added to an amount of, e.g., at least 0.5%, at least 0.55%, at least 0.6%, at least 0.65%, at least 0.7%, at least 0.75%, at least 0.8%, at least 0.85%, at least 0.9%, at least 0.95%, at least 1.0% by weight of the composition. In yet other aspects of this embodiment, a composition disclosed herein has one or more metal salts each added to an amount of, e.g., at most 0.5%, at most 0.55%, at most 0.6%, at most 0.65%, at most 0.7%, at most 0.75%, at most 0.8%, at most 0.85%, at most 0.9%, at most 0.95%, at most 1.0% by weight of the composition. In still other aspects of this embodiment, a composition disclosed herein has one or more metal salts each added to an amount of, e.g., about 0.5% to about 0.6%, about 0.5% to about 0.7%, about 0.5% to about 0.8%, about 0.5% to about 0.9%, about 0.5% to about 1.0%, about 0.6% to about 0.7%, about 0.6% to about 0.8%, about 0.6% to about 0.9%, about 0.6% to about 1.0%, about 0.7% to about 0.8%, about 0.7% to about 0.9%, about 0.7% to about 1.0%, about 0.8% to about 0.9%, about 0.8% to about 1.0%, or about 0.9% to about 1.0% by weight of the composition.

In aspects of this embodiment, a composition disclosed herein has sodium chloride added to an amount of, e.g., about 0.5%, about 0.55%, about 0.6%, about 0.65%, about 0.7%, about 0.75%, about 0.8%, about 0.85%, about 0.9%, about 0.95%, about 1.0% by weight of the composition. In other aspects of this embodiment, a composition disclosed herein has sodium chloride added to an amount of, e.g., at least 0.5%, at least 0.55%, at least 0.6%, at least 0.65%, at least 0.7%, at least 0.75%, at least 0.8%, at least 0.85%, at least 0.9%, at least 0.95%, at least 1.0% by weight of the composition. In yet other aspects of this embodiment, a composition disclosed herein has sodium chloride added to an amount of, e.g., at most 0.5%, at most 0.55%, at most 0.6%, at most 0.65%, at most 0.7%, at most 0.75%, at most 0.8%, at most 0.85%, at most 0.9%, at most 0.95%, at most 1.0% by weight of the composition. In still other aspects of this embodiment, a composition disclosed herein has sodium chloride added to an amount of, e.g., about 0.5% to about 0.6%, about 0.5% to about 0.7%, about 0.5% to about 0.8%, about 0.5% to about 0.9%, about 0.5% to about 1.0%, about 0.6% to about 0.7%, about 0.6% to about 0.8%, about 0.6% to about 0.9%, about 0.6% to about 1.0%, about 0.7% to about 0.8%, about 0.7% to about 0.9%, about 0.7% to about 1.0%, about 0.8% to about 0.9%, about 0.8% to about 1.0%, or about 0.9% to about 1.0% by weight of the composition.

In some embodiments, the amount of metal salts added is an amount that provides a desired osmolality to a composition disclosed herein. Osmolality is a unit of concentration based on osmoles and measures the number of dissolved particles (osmoles) per kilogram of solution and is expressed as mOsm/kg of solution. Normal plasma osmolality in large animals, including humans, ranges from about 275 mOsm/kg to about 295 mOsm/kg, and plasma osmolality is aggressively defended by increasing water intake (osmolality >295 mOsm/kg) or promoting free water excretion (osmolality <275 mOsm/kg).

Typically, a composition disclosed herein comprising free available chlorines and/or peroxide agents is manufactured without consideration of osmolality. However, in certain medical applications the tonicity is a critical quality of the product to ensure biological compatibility with the treatment target on or within the human or animal body. An example of the osmolality effect is the induction of coughing to an individual that may inhale a composition with a low mOsmol/kg composition. By raising the osmolality of the solution to a normal physiological range, the coughing effect may be reduced in certain individuals. Furthermore, as disclosed herein when osmolality of a composition disclosed herein was adjusted using one or more metal to be within a normal physiological range, a surprising result was achieved in that such composition shown significant improvement in the stability and efficacy of the composition.

In aspects of these embodiments, a composition disclosed herein has metal salts added to provide a final osmolality of, e.g., about 275 mOsm/kg, about 276 mOsm/kg, about 278 mOsm/kg, about 280 mOsm/kg, about 282 mOsm/kg, about 284 mOsm/kg, about 285 mOsm/kg, about 286 mOsm/kg, about 288 mOsm/kg, about 290 mOsm/kg, about 292 mOsm/kg, about 294 mOsm/kg, or about 295 mOsm/kg. In other aspects of these embodiments, a composition disclosed herein has metal salts added to provide a final osmolality of, e.g., at least 275 mOsm/kg, at least 276 mOsm/kg, at least 278 mOsm/kg, at least 280 mOsm/kg, at least 282 mOsm/kg, at least 284 mOsm/kg, at least 285 mOsm/kg, at least 286 mOsm/kg, at least 288 mOsm/kg, at least 290 mOsm/kg, at least 292 mOsm/kg, at least 294 mOsm/kg, or at least 295 mOsm/kg. In yet other aspects of these embodiments, a composition disclosed herein has metal salts added to provide a final osmolality of, e.g., at most 275 mOsm/kg, at most 276 mOsm/kg, at most 278 mOsm/kg, at most 280 mOsm/kg, at most 282 mOsm/kg, at most 284 mOsm/kg, at most 285 mOsm/kg, at most 286 mOsm/kg, at most 288 mOsm/kg, at most 290 mOsm/kg, at most 292 mOsm/kg, at most 294 mOsm/kg, or at most 295 mOsm/kg.

In still other aspects of these embodiments, a composition disclosed herein has metal salts added to provide a final osmolality of, e.g., about 275 mOsm/kg to about 280 mOsm/kg, about 275 mOsm/kg to about 282 mOsm/kg, about 275 mOsm/kg to about 284 mOsm/kg, about 275 mOsm/kg to about 285 mOsm/kg, about 275 mOsm/kg to about 286 mOsm/kg, about 275 mOsm/kg to about 288 mOsm/kg, about 275 mOsm/kg to about 290 mOsm/kg, about 275 mOsm/kg to about 292 mOsm/kg, about 275 mOsm/kg to about 294 mOsm/kg, about 275 mOsm/kg to about 295 mOsm/kg, about 280 mOsm/kg to about 282 mOsm/kg, about 280 mOsm/kg to about 284 mOsm/kg, about 280 mOsm/kg to about 285 mOsm/kg, about 280 mOsm/kg to about 286 mOsm/kg, about 280 mOsm/kg to about 288 mOsm/kg, about 280 mOsm/kg to about 290 mOsm/kg, about 280 mOsm/kg to about 292 mOsm/kg, about 280 mOsm/kg to about 294 mOsm/kg, about 280 mOsm/kg to about 295 mOsm/kg, about 282 mOsm/kg to about 284 mOsm/kg, about 282 mOsm/kg to about 285 mOsm/kg, about 282 mOsm/kg to about 286 mOsm/kg, about 282 mOsm/kg to about 288 mOsm/kg, about 282 mOsm/kg to about 290 mOsm/kg, about 282 mOsm/kg to about 292 mOsm/kg, about 282 mOsm/kg to about 294 mOsm/kg, about 282 mOsm/kg to about 295 mOsm/kg, about 284 mOsm/kg to about 285 mOsm/kg, about 284 mOsm/kg to about 286 mOsm/kg, about 284 mOsm/kg to about 288 mOsm/kg, about 284 mOsm/kg to about 290 mOsm/kg, about 284 mOsm/kg to about 292 mOsm/kg, about 284 mOsm/kg to about 294 mOsm/kg, about 284 mOsm/kg to about 295 mOsm/kg, about 286 mOsm/kg to about 288 mOsm/kg, about 286 mOsm/kg to about 290 mOsm/kg, about 286 mOsm/kg to about 292 mOsm/kg, about 286 mOsm/kg to about 294 mOsm/kg, about 286 mOsm/kg to about 295 mOsm/kg, about 285 mOsm/kg to about 290 mOsm/kg, about 285 mOsm/kg to about 292 mOsm/kg, about 285 mOsm/kg to about 294 mOsm/kg, about 285 mOsm/kg to about 295 mOsm/kg, or about 290 mOsm/kg to about 295 mOsm/kg.

In some embodiments, the amount of metal salts added is an amount that provides a desired osmolarity to a composition disclosed herein. Osmolarity is the same as osmotic concentration and measures the number of dissolved particles (osmoles) per liter of solution and is expressed as mOsm/L of solution. A composition disclosed herein is typically physiologically isotonic when the osmolarity is in the range of about 300 mOsm/L to about 316 mOsm/L, physiologically hypertonic when the osmolarity is above 316 mOsm/L), or physiologically hypotonic when the osmolarity is below 300 mOsm/L). One kilogram (1 L) of plasma from an adult large animal, including humans, has two components, 70 g of protein and 930 g of plasma water. Accordingly, the osmolality of normal plasma of about 285 mOsm/kg is equivalent to a plasma water osmolarity of about 306 mOsm/L.

In some embodiments, the amount of metal salts added is an amount that provides a desired osmolarity to a composition disclosed herein that is physiologically isotonic. In aspects of these embodiments, a composition disclosed herein has metal salts added to a final osmolarity of, e.g., about 300 mOsm/L, about 302 mOsm/L, about 304 mOsm/L, about 306 mOsm/L, about 308 mOsm/L, about 310 mOsm/L, about 312 mOsm/L, about 314 mOsm/L, or about 316 mOsm/L. In other aspects of these embodiments, a composition disclosed herein has metal salts added to a final osmolarity of, e.g., at least 300 mOsm/L, at least 302 mOsm/L, at least 304 mOsm/L, at least 306 mOsm/L, at least 308 mOsm/L, at least 310 mOsm/L, at least 312 mOsm/L, at least 314 mOsm/L, or at least 316 mOsm/L. In yet other aspects of these embodiments, a composition disclosed herein has metal salts added to a final osmolarity of, e.g., at most 300 mOsm/L, at most 302 mOsm/L, at most 304 mOsm/L, at most 306 mOsm/L, at most 308 mOsm/L, at most 310 mOsm/L, at most 312 mOsm/L, at most 314 mOsm/L, or at most 316 mOsm/L. In still other aspects of these embodiments, a composition disclosed herein has metal salts added to a final osmolarity of, e.g., about 300 mOsm/L to about 302 mOsm/L, about 300 mOsm/L to about 304 mOsm/L, about 300 mOsm/L to about 306 mOsm/L, about 300 mOsm/L to about 308 mOsm/L, about 300 mOsm/L to about 310 mOsm/L, about 300 mOsm/L to about 312 mOsm/L, about 300 mOsm/L to about 314 mOsm/L, about 300 mOsm/L to about 316 mOsm/L, about 302 mOsm/L to about 304 mOsm/L, about 302 mOsm/L to about 306 mOsm/L, about 302 mOsm/L to about 308 mOsm/L, about 302 mOsm/L to about 310 mOsm/L, about 302 mOsm/L to about 312 mOsm/L, about 302 mOsm/L to about 314 mOsm/L, about 302 mOsm/L to about 316 mOsm/L, about 304 mOsm/L to about 306 mOsm/L, about 304 mOsm/L to about 308 mOsm/L, about 304 mOsm/L to about 310 mOsm/L, about 304 mOsm/L to about 312 mOsm/L, about 304 mOsm/L to about 314 mOsm/L, about 304 mOsm/L to about 316 mOsm/L, about 306 mOsm/L to about 308 mOsm/L, about 306 mOsm/L to about 310 mOsm/L, about 306 mOsm/L to about 312 mOsm/L, about 306 mOsm/L to about 314 mOsm/L, about 306 mOsm/L to about 316 mOsm/L, about 308 mOsm/L to about 310 mOsm/L, about 308 mOsm/L to about 312 mOsm/L, about 308 mOsm/L to about 314 mOsm/L, about 308 mOsm/L to about 316 mOsm/L, about 310 mOsm/L to about 312 mOsm/L, about 310 mOsm/L to about 314 mOsm/L, about 310 mOsm/L to about 316 mOsm/L, about 312 mOsm/L to about 314 mOsm/L, about 312 mOsm/L to about 316 mOsm/L, or about 314 mOsm/L to about 316 mOsm/L.

In some embodiments, the amount of metal salts added is an amount that provides a desired osmolarity to a composition disclosed herein that is physiologically hypotonic. In aspects of these embodiments, a composition disclosed herein has metal salts added to a final osmolarity of, e.g., about 298 mOsm/L, about 296 mOsm/L, about 294 mOsm/L, about 292 mOsm/L, about 290 mOsm/L, about 288 mOsm/L, about 286 mOsm/L, about 284 mOsm/L, about 282 mOsm/L, or about 280 mOsm/L. In other aspects of these embodiments, a composition disclosed herein has metal salts added to a final osmolarity of, e.g., at least 298 mOsm/L, at least 296 mOsm/L, at least 294 mOsm/L, at least 292 mOsm/L, at least 290 mOsm/L, at least 288 mOsm/L, at least 286 mOsm/L, at least 284 mOsm/L, at least 282 mOsm/L, or at least 280 mOsm/L. In yet other aspects of these embodiments, a composition disclosed herein has metal salts added to a final osmolarity of, e.g., at most 298 mOsm/L, at most 296 mOsm/L, at most 294 mOsm/L, at most 292 mOsm/L, at most 290 mOsm/L, at most 288 mOsm/L, at most 286 mOsm/L, at most 284 mOsm/L, at most 282 mOsm/L, or at most 280 mOsm/L. In yet other aspects of these embodiments, a composition disclosed herein has metal salts added to a final osmolarity of, e.g., about 280 mOsm/L to about 284 mOsm/L, about 280 mOsm/L to about 288 mOsm/L, about 280 mOsm/L to about 292 mOsm/L, about 280 mOsm/L to about 296 mOsm/L, about 280 mOsm/L to about 299 mOsm/L, about 284 mOsm/L to about 288 mOsm/L, about 284 mOsm/L to about 292 mOsm/L, about 284 mOsm/L to about 296 mOsm/L, about 284 mOsm/L to about 299 mOsm/L, about 288 mOsm/L to about 292 mOsm/L, about 288 mOsm/L to about 296 mOsm/L, about 288 mOsm/L to about 299 mOsm/L, about 292 mOsm/L to about 296 mOsm/L, about 292 mOsm/L to about 299 mOsm/L, or about 296 mOsm/L to about 299 mOsm/L.

In some embodiments, the amount of metal salts added is an amount that provides a desired osmolarity to a composition disclosed herein that is physiologically hypertonic. In aspects of these embodiments, a composition disclosed herein has metal salts added to a final osmolarity of, e.g., about 318 mOsm/L, about 320 mOsm/L, about 322 mOsm/L, about 324 mOsm/L, about 326 mOsm/L, about 328 mOsm/L, about 330 mOsm/L, about 332 mOsm/L, about 334 mOsm/L, or about 336 mOsm/L. In other aspects of these embodiments, a composition disclosed herein has metal salts added to a final osmolarity of, e.g., at least 318 mOsm/L, at least 320 mOsm/L, at least 322 mOsm/L, at least 324 mOsm/L, at least 326 mOsm/L, at least 328 mOsm/L, at least 330 mOsm/L, at least 332 mOsm/L, at least 334 mOsm/L, or at least 336 mOsm/L. In yet other aspects of these embodiments, a composition disclosed herein has metal salts added to a final osmolarity of, e.g., at most 318 mOsm/L, at most 320 mOsm/L, at most 322 mOsm/L, at most 324 mOsm/L, at most 326 mOsm/L, at most 328 mOsm/L, at most 330 mOsm/L, at most 332 mOsm/L, at most 334 mOsm/L, or at most 336 mOsm/L. In still other aspects of these embodiments, a composition disclosed herein has metal salts added to a final osmolarity of, e.g., about 317 mOsm/L to at most 322 mOsm/L, about 317 mOsm/L to at most 326 mOsm/L, about 317 mOsm/L to at most 330 mOsm/L, about 317 mOsm/L to at most 334 mOsm/L, about 317 mOsm/L to at most 338 mOsm/L, about 322 mOsm/L to at most 326 mOsm/L, about 322 mOsm/L to at most 330 mOsm/L, about 322 mOsm/L to at most 334 mOsm/L, about 322 mOsm/L to at most 338 mOsm/L, about 326 mOsm/L to at most 330 mOsm/L, about 326 mOsm/L to at most 334 mOsm/L, about 326 mOsm/L to at most 338 mOsm/L, about 330 mOsm/L to at most 334 mOsm/L, about 330 mOsm/L to at most 338 mOsm/L, or about 334 mOsm/L to at most 338 mOsm/L.

In an embodiment, a composition disclosed herein does not comprise metal salts.

In some embodiments, a phosphate buffer can be added to a composition manufactured according to a method or use disclosed herein. A phosphate buffer or phosphate buffer solution) is especially sensitive to pH changes, and thus is useful for biological applications. A phosphate buffer can be adapted to a variety of pH levels, including isotonic. This wide range is due to phosphoric acid having 3 dissociation constants (pKa) allowing for formulation of buffers near each of the pH levels of 2.15, 7.21, or 12.32. Monosodium phosphate and its conjugate base, disodium phosphate, are usually used to generate buffers of pH values around 7. Non-limiting examples of a phosphate buffer include a calcium phosphate buffer and a sodium phosphate buffer.

The amount of phosphate buffer added is an amount that provides a desired beneficial effect to a composition disclosed herein. In aspects of this embodiment, a composition disclosed herein has a phosphate buffer added to a final concentration of, e.g., 0.05 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 825 ppm, 850 ppm, 875 ppm, 900 ppm, 925 ppm, 950 ppm, 975 ppm, 1,000 ppm, 1,025 ppm, 1,050 ppm, 1075 ppm, 1,100 ppm, 1,125 ppm, 1,150 ppm, 1,175 ppm, 1,200 ppm, 1,225 ppm, 1,250 ppm, 1,275 ppm, 1,300 ppm, 1,325 ppm, 1,350 ppm, 1,375 ppm, 1,400 ppm, 1,425 ppm, 1,450 ppm, 1,475 ppm, 1,500 ppm, 1,600 ppm, 1,700 ppm, 1,800 ppm, 1,900 ppm, or 2,000 ppm. In other aspects of this embodiment, a composition disclosed herein has a phosphate buffer added to a final concentration of, e.g., at least 0.05 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 10 ppm, at least 20 ppm, at least 30 ppm, at least 40 ppm, at least 50 ppm, at least 60 ppm, at least 70 ppm, at least 80 ppm, at least 90 ppm, at least 100 ppm, at least 125 ppm, at least 150 ppm, at least 175 ppm, at least 200 ppm, at least 225 ppm, at least 250 ppm, at least 275 ppm, at least 300 ppm, at least 325 ppm, at least 350 ppm, at least 375 ppm, at least 400 ppm, at least 425 ppm, at least 450 ppm, at least 475 ppm, at least 500 ppm, at least 525 ppm, at least 550 ppm, at least 575 ppm, at least 600 ppm, at least 625 ppm, at least 650 ppm, at least 675 ppm, at least 700 ppm, at least 725 ppm, at least 750 ppm, at least 775 ppm, at least 800 ppm, at least 825 ppm, at least 850 ppm, at least 875 ppm, at least 900 ppm, at least 925 ppm, at least 950 ppm, at least 975 ppm, at least 1,000 ppm, at least 1,025 ppm, at least 1,050 ppm, at least 1075 ppm, at least 1,100 ppm, at least 1,125 ppm, at least 1,150 ppm, at least 1,175 ppm, at least 1,200 ppm, at least 1,225 ppm, at least 1,250 ppm, at least 1,275 ppm, at least 1,300 ppm, at least 1,325 ppm, at least 1,350 ppm, at least 1,375 ppm, at least 1,400 ppm, at least 1,425 ppm, at least 1,450 ppm, at least 1,475 ppm, at least 1,500 ppm, at least 1,600 ppm, at least 1,700 ppm, at least 1,800 ppm, at least 1,900 ppm, or at least 2,000 ppm. In yet other aspects of this embodiment, a composition disclosed herein has a phosphate buffer added to a final concentration of, e.g., at most 0.05 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 125 ppm, at most 150 ppm, at most 175 ppm, at most 200 ppm, at most 225 ppm, at most 250 ppm, at most 275 ppm, at most 300 ppm, at most 325 ppm, at most 350 ppm, at most 375 ppm, at most 400 ppm, at most 425 ppm, at most 450 ppm, at most 475 ppm, at most 500 ppm, at most 525 ppm, at most 550 ppm, at most 575 ppm, at most 600 ppm, at most 625 ppm, at most 650 ppm, at most 675 ppm, at most 700 ppm, at most 725 ppm, at most 750 ppm, at most 775 ppm, at most 800 ppm, at most 825 ppm, at most 850 ppm, at most 875 ppm, at most 900 ppm, at most 925 ppm, at most 950 ppm, at most 975 ppm, at most 1,000 ppm, at most 1,025 ppm, at most 1,050 ppm, at most 1075 ppm, at most 1,100 ppm, at most 1,125 ppm, at most 1,150 ppm, at most 1,175 ppm, at most 1,200 ppm, at most 1,225 ppm, at most 1,250 ppm, at most 1,275 ppm, at most 1,300 ppm, at most 1,325 ppm, at most 1,350 ppm, at most 1,375 ppm, at most 1,400 ppm, at most 1,425 ppm, at most 1,450 ppm, at most 1,475 ppm, at most 1,500 ppm, at most 1,600 ppm, at most 1,700 ppm, at most 1,800 ppm, at most 1,900 ppm, or at most 2,000 ppm.

In still other aspects of this embodiment, a composition disclosed herein has a phosphate buffer added to a final concentration of, e.g., about 0.5 ppm to about 20 ppm, about 0.5 ppm to about 25 ppm, about 0.5 ppm to about 30 ppm, about 0.5 ppm to about 35 ppm, about 0.5 ppm to about 40 ppm, about 0.5 ppm to about 45 ppm, about 0.5 ppm to about 50 ppm, about 0.5 ppm to about 55 ppm, about 0.5 ppm to about 60 ppm, about 0.5 ppm to about 65 ppm, about 0.5 ppm to about 70 ppm, about 0.5 ppm to about 75 ppm, about 0.5 ppm to about 80 ppm, about 0.5 ppm to about 85 ppm, about 0.5 ppm to about 90 ppm, about 0.5 ppm to about 95 ppm, about 0.5 ppm to about 100 ppm, about 0.75 ppm to about 20 ppm, about 0.75 ppm to about 25 ppm, about 0.75 ppm to about 30 ppm, about 0.75 ppm to about 35 ppm, about 0.75 ppm to about 40 ppm, about 0.75 ppm to about 45 ppm, about 0.75 ppm to about 50 ppm, about 0.75 ppm to about 55 ppm, about 0.75 ppm to about 60 ppm, about 0.75 ppm to about 65 ppm, about 0.75 ppm to about 70 ppm, about 0.75 ppm to about 75 ppm, about 0.75 ppm to about 80 ppm, about 0.75 ppm to about 85 ppm, about 0.75 ppm to about 90 ppm, about 0.75 ppm to about 95 ppm, about 0.75 ppm to about 100 ppm, about 1 ppm to about 20 ppm, about 1 ppm to about 25 ppm, about 1 ppm to about 30 ppm, about 1 ppm to about 35 ppm, about 1 ppm to about 40 ppm, about 1 ppm to about 45 ppm, about 1 ppm to about 50 ppm, about 1 ppm to about 55 ppm, about 1 ppm to about 60 ppm, about 1 ppm to about 65 ppm, about 1 ppm to about 70 ppm, about 1 ppm to about 75 ppm, about 1 ppm to about 80 ppm, about 1 ppm to about 85 ppm, about 1 ppm to about 90 ppm, about 1 ppm to about 95 ppm, about 1 ppm to about 100 ppm, about 5 ppm to about 20 ppm, about 5 ppm to about 25 ppm, about 5 ppm to about 30 ppm, about 5 ppm to about 35 ppm, about 5 ppm to about 40 ppm, about 5 ppm to about 45 ppm, about 5 ppm to about 50 ppm, about 5 ppm to about 55 ppm, about 5 ppm to about 60 ppm, about 5 ppm to about 65 ppm, about 5 ppm to about 70 ppm, about 5 ppm to about 75 ppm, about 5 ppm to about 80 ppm, about 5 ppm to about 85 ppm, about 5 ppm to about 90 ppm, about 5 ppm to about 95 ppm, about 5 ppm to about 100 ppm, about 10 ppm to about 20 ppm, about 10 ppm to about 25 ppm, about 10 ppm to about 30 ppm, about 10 ppm to about 35 ppm, about 10 ppm to about 40 ppm, about 10 ppm to about 45 ppm, about 10 ppm to about 50 ppm, about 10 ppm to about 55 ppm, about 10 ppm to about 60 ppm, about 10 ppm to about 65 ppm, about 10 ppm to about 70 ppm, about 10 ppm to about 75 ppm, about 10 ppm to about 80 ppm, about 10 ppm to about 85 ppm, about 10 ppm to about 90 ppm, about 10 ppm to about 95 ppm, or about 10 ppm to about 100 ppm.

In other aspects of this embodiment, a composition disclosed herein has a phosphate buffer added to a final concentration of, e.g., about 1 ppm to about 25 ppm, about 1 ppm to about 50 ppm, about 1 ppm to about 75 ppm, about 1 ppm to about 100 ppm, about 1 ppm to about 125 ppm, about 1 ppm to about 150 ppm, about 1 ppm to about 175 ppm, about 1 ppm to about 200 ppm, about 1 ppm to about 225 ppm, about 1 ppm to about 250 ppm, about 1 ppm to about 275 ppm, about 1 ppm to about 300 ppm, about 1 ppm to about 325 ppm, about 1 ppm to about 350 ppm, about 1 ppm to about 375 ppm, about 1 ppm to about 400 ppm, about 1 ppm to about 425 ppm, about 1 ppm to about 450 ppm, about 1 ppm to about 475 ppm, about 1 ppm to about 500 ppm, about 5 ppm to about 25 ppm, about 5 ppm to about 50 ppm, about 5 ppm to about 75 ppm, about 5 ppm to about 100 ppm, about 5 ppm to about 125 ppm, about 5 ppm to about 150 ppm, about 5 ppm to about 175 ppm, about 5 ppm to about 200 ppm, about 5 ppm to about 225 ppm, about 5 ppm to about 250 ppm, about 5 ppm to about 275 ppm, about 1 ppm to about 300 ppm, about 5 ppm to about 325 ppm, about 5 ppm to about 350 ppm, about 5 ppm to about 375 ppm, about 5 ppm to about 400 ppm, about 5 ppm to about 425 ppm, about 5 ppm to about 450 ppm, about 5 ppm to about 475 ppm, about 5 ppm to about 500 ppm, about 10 ppm to about 25 ppm, about 10 ppm to about 50 ppm, about 10 ppm to about 75 ppm, about 10 ppm to about 100 ppm, about 10 ppm to about 125 ppm, about 10 ppm to about 150 ppm, about 10 ppm to about 175 ppm, about 10 ppm to about 200 ppm, about 10 ppm to about 225 ppm, about 10 ppm to about 250 ppm, about 10 ppm to about 275 ppm, about 10 ppm to about 300 ppm, about 10 ppm to about 325 ppm, about 10 ppm to about 350 ppm, about 10 ppm to about 375 ppm, about 10 ppm to about 400 ppm, about 10 ppm to about 425 ppm, about 10 ppm to about 450 ppm, about 10 ppm to about 475 ppm, about 10 ppm to about 500 ppm, about 25 ppm to about 50 ppm, about 25 ppm to about 75 ppm, about 25 ppm to about 100 ppm, about 25 ppm to about 125 ppm, about 25 ppm to about 150 ppm, about 25 ppm to about 175 ppm, about 25 ppm to about 200 ppm, about 25 ppm to about 225 ppm, about 25 ppm to about 250 ppm, about 25 ppm to about 275 ppm, about 25 ppm to about 300 ppm, about 25 ppm to about 325 ppm, about 25 ppm to about 350 ppm, about 25 ppm to about 375 ppm, about 25 ppm to about 400 ppm, about 25 ppm to about 425 ppm, about 25 ppm to about 450 ppm, about 25 ppm to about 475 ppm, about 25 ppm to about 500 ppm, about 50 ppm to about 75 ppm, about 50 ppm to about 100 ppm, about 50 ppm to about 125 ppm, about 50 ppm to about 150 ppm, about 50 ppm to about 175 ppm, about 50 ppm to about 200 ppm, about 50 ppm to about 225 ppm, about 50 ppm to about 250 ppm, about 50 ppm to about 275 ppm, about 50 ppm to about 300 ppm, about 50 ppm to about 325 ppm, about 50 ppm to about 350 ppm, about 50 ppm to about 375 ppm, about 50 ppm to about 400 ppm, about 50 ppm to about 425 ppm, about 50 ppm to about 450 ppm, about 50 ppm to about 475 ppm, about 50 ppm to about 500 ppm, about 75 ppm to about 100 ppm, about 75 ppm to about 125 ppm, about 75 ppm to about 150 ppm, about 75 ppm to about 175 ppm, about 75 ppm to about 200 ppm, about 75 ppm to about 225 ppm, about 75 ppm to about 250 ppm, about 75 ppm to about 275 ppm, about 75 ppm to about 300 ppm, about 75 ppm to about 325 ppm, about 75 ppm to about 350 ppm, about 75 ppm to about 375 ppm, about 75 ppm to about 400 ppm, about 75 ppm to about 425 ppm, about 75 ppm to about 450 ppm, about 75 ppm to about 475 ppm, about 75 ppm to about 500 ppm, about 100 ppm to about 125 ppm, about 100 ppm to about 150 ppm, about 100 ppm to about 175 ppm, about 100 ppm to about 200 ppm, about 100 ppm to about 225 ppm, about 100 ppm to about 250 ppm, about 100 ppm to about 275 ppm, about 100 ppm to about 300 ppm, about 100 ppm to about 325 ppm, about 100 ppm to about 350 ppm, about 100 ppm to about 375 ppm, about 100 ppm to about 400 ppm, about 100 ppm to about 425 ppm, about 100 ppm to about 450 ppm, about 100 ppm to about 475 ppm, about 100 ppm to about 500 ppm, about 150 ppm to about 175 ppm, about 150 ppm to about 200 ppm, about 150 ppm to about 225 ppm, about 150 ppm to about 250 ppm, about 150 ppm to about 275 ppm, about 150 ppm to about 300 ppm, about 150 ppm to about 325 ppm, about 150 ppm to about 350 ppm, about 150 ppm to about 375 ppm, about 150 ppm to about 400 ppm, about 150 ppm to about 425 ppm, about 150 ppm to about 450 ppm, about 150 ppm to about 475 ppm, about 150 ppm to about 500 ppm, about 200 ppm to about 225 ppm, about 200 ppm to about 250 ppm, about 200 ppm to about 275 ppm, about 200 ppm to about 300 ppm, about 200 ppm to about 325 ppm, about 200 ppm to about 350 ppm, about 200 ppm to about 375 ppm, about 200 ppm to about 400 ppm, about 200 ppm to about 425 ppm, about 200 ppm to about 450 ppm, about 200 ppm to about 475 ppm, about 200 ppm to about 500 ppm, about 250 ppm to about 275 ppm, about 250 ppm to about 300 ppm, about 250 ppm to about 325 ppm, about 250 ppm to about 350 ppm, about 250 ppm to about 375 ppm, about 250 ppm to about 400 ppm, about 250 ppm to about 425 ppm, about 250 ppm to about 450 ppm, about 250 ppm to about 475 ppm, about 250 ppm to about 500 ppm, about 300 ppm to about 325 ppm, about 300 ppm to about 350 ppm, about 300 ppm to about 375 ppm, about 300 ppm to about 400 ppm, about 300 ppm to about 425 ppm, about 300 ppm to about 450 ppm, about 300 ppm to about 475 ppm, about 300 ppm to about 500 ppm, about 350 ppm to about 375 ppm, about 350 ppm to about 400 ppm, about 350 ppm to about 425 ppm, about 350 ppm to about 450 ppm, about 350 ppm to about 475 ppm, about 350 ppm to about 500 ppm, about 400 ppm to about 425 ppm, about 400 ppm to about 450 ppm, about 400 ppm to about 475 ppm, about 400 ppm to about 500 ppm, about 450 ppm to about 475 ppm, or about 450 ppm to about 500 ppm.

In an embodiment, a composition disclosed herein does not comprise a phosphate buffer. In an embodiment, a composition disclosed herein does not comprise a calcium phosphate buffer. In an embodiment, a composition disclosed herein does not comprise a sodium phosphate buffer.

In one or more embodiments, a microbiostatic effective amount of a compound disclosed herein, such as, e.g., a peroxide, a hydrogen peroxide, a sodium peroxide, a stable free available chlorine, a stable hypochlorous acid, a stable hypochlorite, a metallic particle, and a metal salt, is present in a composition disclosed herein and/or applied in a method or use disclosed herein.

In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is an amount that 1) restrains, prohibits or otherwise prevents growth of a microbial population; 2) results in no more than a 1×10^{0.5}, a 1×10¹ or a 1×10² reduction in the microbial population within the first seven days of continuous exposure to the microbiostatic agent; and/or 3) results in a minimum of a 1×10^{0.5}, a 1×10¹ or a 1×10² reduction in the microbial population after at least 14 days of continuous exposure to the microbiostatic agent without achieving full kill of the microbial population. In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is an amount that 1) restrains, prohibits or otherwise prevents growth of a microbial population; 2) results in no more than a 1×10^{0.5}, a 1×10¹ or a 1×10² reduction in the microbial population within the first seven days of continuous exposure to the microbiostatic agent; and/or 3) results in a minimum of a 1×10^{0.5}, a 1×10¹ or 1×10² reduction in the microbial population after at least 14 days of continuous exposure to the microbiostatic agent but no more than 1×10^{0.5} reduction of the microbial population, no more than 1×10¹ reduction of the microbial population, no more than 1×10² reduction of the microbial population, no more than 1×10² reduction of the microbial population, no more than 1×10⁴ reduction of the microbial population, no more than 1×10⁵ reduction of the microbial population, no more than 1×10⁶ reduction of the microbial population, or no more than 1×10⁷ reduction of the microbial population.

In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is an amount that 1) restrains, prohibits or otherwise prevents growth of a microbial population; 2) results in about 1×10° to about 1×10², about 1×10^{0.5} to about 1×10², or about 1×10¹ to about 1×10² reduction in the microbial population within the first seven days of continuous exposure to the microbiostatic agent; and/or 3) results in a minimum of a 1×10° to 1×10², a 1×10^{0.5} to 1×10², or a 1×10¹ to 1×10² reduction in the microbial population after at least 14 days of continuous exposure to the microbiostatic agent without achieving full kill of the microbial population. In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is an amount that 1) restrains, prohibits or otherwise prevents growth of a microbial population; 2) results in about 1×10° to about 1×10², about 1×10^{0.5} to about 1×10², or about 1×10¹ to about 1×10² reduction in the microbial population within the first seven days of continuous exposure to the microbiostatic agent; and/or 3) results in a minimum of a 1×10² reduction in the microbial population after at least 14 days of continuous exposure to the microbiostatic agent but no more than 1×10^{0.5} reduction of the microbial population, no more than 1×10¹ reduction of the microbial population, no more than 1×10² reduction of the microbial population, no more than 1×10² reduction of the microbial population, no more than 1×10⁴ reduction of the microbial population, no more than 1×10⁵ reduction of the microbial population, no more than 1×10⁶ reduction of the microbial population, or no more than 1×10⁷ reduction of the microbial population.

In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is in a final concentration of, e.g., 0.01 ppm, 0.025 ppm, 0.05 ppm, 0.075 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 2 ppm, 3 ppm, 4 ppm, 5 ppm. In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is in a final concentration of, e.g., at least 0.01 ppm, at least 0.025 ppm, at least 0.05 ppm, at least 0.075 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 2 ppm, at least 3 ppm, at least 4 ppm, at least 5 ppm. In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is in a final concentration of, e.g., at most 0.01 ppm, at most 0.025 ppm, at most 0.05 ppm, at most 0.075 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 2 ppm, at most 3 ppm, at most 4 ppm, at most 5 ppm.

In some embodiments, a microbiostatic effective amount of one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, is in a final concentration of between, e.g., about 0.01 ppm to about 0.025 ppm, about 0.01 ppm to about 0.05 ppm, about 0.01 ppm to about 0.075 ppm, about 0.01 ppm to about 0.1 ppm, about 0.01 ppm to about 0.25 ppm, about 0.01 ppm to about 0.5 ppm, about 0.01 ppm to about 0.75 ppm, about 0.01 ppm to about 1 ppm, about 0.01 ppm to about 2 ppm, about 0.01 ppm to about 3 ppm, about 0.01 ppm to about 4 ppm, about 0.01 ppm to about 5 ppm, about 0.025 ppm to about 0.075 ppm, about 0.025 ppm to about 0.1 ppm, about 0.025 ppm to about 0.25 ppm, about 0.025 ppm to about 0.5 ppm, about 0.025 ppm to about 0.75 ppm, about 0.025 ppm to about 1 ppm, about 0.025 ppm to about 2 ppm, about 0.025 ppm to about 3 ppm, about 0.025 ppm to about 4 ppm, about 0.025 ppm to about 5 ppm, about 0.05 ppm to about 0.075 ppm, about 0.05 ppm to about 0.1 ppm, about 0.05 ppm to about 0.25 ppm, about 0.05 ppm to about 0.5 ppm, about 0.05 ppm to about 0.75 ppm, about 0.05 ppm to about 1 ppm, about 0.05 ppm to about 2 ppm, about 0.05 ppm to about 3 ppm, about 0.05 ppm to about 4 ppm, about 0.05 ppm to about 5 ppm, about 0.075 ppm to about 0.1 ppm, about 0.075 ppm to about 0.25 ppm, about 0.075 ppm to about 0.5 ppm, about 0.075 ppm to about 0.75 ppm, about 0.075 ppm to about 1 ppm, about 0.075 ppm to about 2 ppm, about 0.075 ppm to about 3 ppm, about 0.075 ppm to about 4 ppm, about 0.075 ppm to about 5 ppm, about 0.1 ppm to about 0.25 ppm, about 0.1 ppm to about 0.5 ppm, about 0.1 ppm to about 0.75 ppm, about 0.1 ppm to about 1 ppm, about 0.1 ppm to about 2 ppm, about 0.1 ppm to about 3 ppm, about 0.1 ppm to about 4 ppm, or about 0.1 ppm to about 5 ppm.

In some embodiments, a final concentration of a microbiostatic effective amount is the concentration of all of the one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, that are present. In some embodiments, a final concentration of all of the one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, present as a microbiostatic effective amount is, e.g., 0.01 ppm, 0.025 ppm, 0.05 ppm, 0.075 ppm, 0.10 ppm, 0.15 ppm, 0.20 ppm, 0.25 ppm, 0.30 ppm, 0.35 ppm, 0.40 ppm, 0.45 ppm, 0.50 ppm, 0.55 ppm, 0.60 ppm, 0.65 ppm, 0.70 ppm, 0.75 ppm, 0.80 ppm, 0.85 ppm, 0.90 ppm, 0.95 ppm, 1 ppm, 2 ppm, 3 ppm, 4 ppm, 5 ppm. In some embodiments, a final concentration of all of the one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, present as a microbiostatic effective amount is, e.g., at least 0.01 ppm, at least 0.025 ppm, at least 0.05 ppm, at least 0.075 ppm, at least 0.10 ppm, at least 0.20 ppm, at least 0.30 ppm, at least 0.40 ppm, at least 0.50 ppm, at least 0.60 ppm, at least 0.70 ppm, at least 0.80 ppm, at least 0.90 ppm, at least 1 ppm, at least 2 ppm, at least 3 ppm, at least 4 ppm, at least 5 ppm. In some embodiments, a final concentration of all of the one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, present as a microbiostatic effective amount is, e.g., at most 0.01 ppm, at most 0.025 ppm, at most 0.05 ppm, at most 0.075 ppm, at most 0.10 ppm, at most 0.20 ppm, at most 0.30 ppm, at most 0.40 ppm, at most 0.50 ppm, at most 0.60 ppm, at most 0.70 ppm, at most 0.80 ppm, at most 0.90 ppm, at most 1 ppm, at most 2 ppm, at most 3 ppm, at most 4 ppm, at most 5 ppm.

In some embodiments, a final concentration of all of the one or more compounds disclosed herein, such as, e.g., a peroxide, a hydrogen peroxide, a sodium peroxide, a stable free available chlorine, a stable hypochlorous acid, a stable hypochlorite, a metallic particle, and a metal salt, present as a microbiostatic effective amount is between, e.g., about 0.01 ppm to about 0.025 ppm, about 0.01 ppm to about 0.05 ppm, about 0.01 ppm to about 0.075 ppm, about 0.01 ppm to about 0.1 ppm, about 0.01 ppm to about 0.25 ppm, about 0.01 ppm to about 0.5 ppm, about 0.01 ppm to about 0.75 ppm, about 0.01 ppm to about 1 ppm, about 0.01 ppm to about 2 ppm, about 0.01 ppm to about 3 ppm, about 0.01 ppm to about 4 ppm, about 0.01 ppm to about 5 ppm, about 0.025 ppm to about 0.075 ppm, about 0.025 ppm to about 0.1 ppm, about 0.025 ppm to about 0.25 ppm, about 0.025 ppm to about 0.5 ppm, about 0.025 ppm to about 0.75 ppm, about 0.025 ppm to about 1 ppm, about 0.025 ppm to about 2 ppm, about 0.025 ppm to about 3 ppm, about 0.025 ppm to about 4 ppm, about 0.025 ppm to about 5 ppm, about 0.05 ppm to about 0.075 ppm, about 0.05 ppm to about 0.1 ppm, about 0.05 ppm to about 0.25 ppm, about 0.05 ppm to about 0.5 ppm, about 0.05 ppm to about 0.75 ppm, about 0.05 ppm to about 1 ppm, about 0.05 ppm to about 2 ppm, about 0.05 ppm to about 3 ppm, about 0.05 ppm to about 4 ppm, about 0.05 ppm to about 5 ppm, about 0.075 ppm to about 0.1 ppm, about 0.075 ppm to about 0.25 ppm, about 0.075 ppm to about 0.5 ppm, about 0.075 ppm to about 0.75 ppm, about 0.075 ppm to about 1 ppm, about 0.075 ppm to about 2 ppm, about 0.075 ppm to about 3 ppm, about 0.075 ppm to about 4 ppm, about 0.075 ppm to about 5 ppm, about 0.1 ppm to about 0.25 ppm, about 0.1 ppm to about 0.5 ppm, about 0.1 ppm to about 0.75 ppm, about 0.1 ppm to about 1 ppm, about 0.1 ppm to about 2 ppm, about 0.1 ppm to about 3 ppm, about 0.1 ppm to about 4 ppm, or about 0.1 ppm to about 5 ppm.

A composition disclosed herein may be adjusted to any pH that enables a composition disclosed herein to achieve a desired beneficial effect. A composition disclosed herein can be pH using a strong acid like hydrochloric acid or base like sodium hydroxide, or a weak acid like acetic acid, citric acid, or phosphoric acid, or a weak base, depending on the desired pH. In aspects of this embodiment, the pH of a composition disclosed herein is, e.g., at least 2, at least 3, at least 4, at least 5, at least 6, at least 7, at least 8, at least 9, at least 10, or at least 11. In other aspects of this embodiment, the pH of a composition disclosed herein is, e.g., at most 2, at most 3, at most 4, at most 5, at most 6, at most 7, at most 8, at most 9, at most 10, or at most 11.

In yet other aspects of this embodiment, the pH of a composition disclosed herein is between, e.g., about 2 to about 5, about 2 to about 5.5, about 2 to about 6, about 2 to about 6.5, about 2 to about 7, about 2 to about 7.5, about 2 to about 8, about 2 to about 8.5, about 2 to about 9, about 2.5 to about 5, about 2.5 to about 5.5, about 2.5 to about 6, about 2.5 to about 6.5, about 2.5 to about 7, about 2.5 to about 7.5, about 2.5 to about 8, about 2.5 to about 8.5, about 2.5 to about 9, about 3 to about 5, about 3 to about 5.5, about 3 to about 6, about 3 to about 6.5, about 3 to about 7, about 3 to about 7.5, about 3 to about 8, about 3 to about 8.5, about 3 to about 9, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 8.5, about 3.5 to about 9, about 4 to about 5, about 4 to about 5.5, about 4 to about 6, about 4 to about 6.5, about 4 to about 7, about 4 to about 7.5, about 4 to about 8, about 4 to about 8.5, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 8.5, about 4.5 to about 9, about 5 to about 5.5, about 5 to about 6, about 5 to about 6.5, about 5 to about 7, about 5 to about 7.5, about 5 to about 8, about 5 to about 8.5, about 5 to about 9, about 5.5 to about 6, about 5.5 to about 6.5, about 5.5 to about 7, about 5.5 to about 7.5, about 5.5 to about 8, about 5.5 to about 8.5, about 5.5 to about 9, about 6 to about 6.5, about 6 to about 7, about 6 to about 7.5, about 6 to about 8, about 6 to about 8.5, about 6 to about 9, about 6.5 to about 7, about 6.5 to about 7.5, about 6.5 to about 8, about 6.5 to about 8.5, about 6.5 to about 9, about 7 to about 7.5, about 7 to about 8, about 7 to about 8.5, about 7 to about 9, about 7.5 to about 8, about 7.5 to about 8.5, about 7.5 to about 9, about 8 to about 8.5, about 8 to about 9, or about 8.5 to about 9.

Aspects of the present specification disclose a kit. In one embodiment, the kit can comprise a container that includes a composition disclosed herein. In another embodiment, a kit can comprise a plurality of containers, with each such container including a composition disclosed herein. For example, a kit can comprise 1, 2, 3, 4, 5, 6, 7, 8, 9, 10, 11, 12, 13, 14, 15, 16, 17, 18, 19 or 20 or more containers, with each such container including a composition disclosed herein. Each of the disclosed containers can comprise a single dose of a composition disclosed herein, multiple doses of a composition disclosed herein, or a combination thereof. In addition, each of the disclosed containers can contain a composition disclosed herein in liquid form or in dried form.

A kit disclosed herein can comprise a delivery or application system. The delivery or application system of the kit are useful for applying one or more compositions disclosed herein, and/or individual components disclosed herein to a site of interest. A delivery or application system disclosed herein includes, without limitation, one or more of an applicator brushes, porous foam swab or pad, hollow tube, dipstick and the like or a combination thereof. In an embodiment, a kit comprises a single delivery or application system. In another embodiment, a kit comprises a plurality delivery or application systems. For example, in a 30-day supply kit, there can be 30 delivery or application systems, such that there is one delivery or application system per day for 30 days. Alternately, there can be 2, 10, 20, 30, 40, 50, 60, 90, 120, etc. delivery or application systems per kit. Within the kit, the delivery or application system may be packaged individually, or in sets of 2 or more. The delivery or application system can be packaged such that it remains sterile until use. In certain embodiments, a delivery or application system disclosed herein can be packaged in plastic sheaths. Further, to prevent contamination, delivery or application system disclosed herein is preferably single-use, disposable delivery, or application system.

The kit can also comprise a set of instructions. The instructions may include information useful to the end user such as, e.g., how to use a delivery system to apply a composition disclosed herein and/or how often to apply a composition disclosed herein. In addition, such instructions may include information regarding how to mix the individual components disclosed herein to form a composition disclosed herein, if provided as individual components to be combined before use. Such instructions can indicate that mixing should be done at a certain time before application, such as, e.g., just prior to use. Instructions disclosed herein may also include information regarding how to apply the individual components disclosed herein directly to a site of interest, and in what order the individual components should be applied to such sites of interest if multiple compositions are utilized as part of a regimen, for example.

A kit disclosed herein can comprise other components. For example, a kit disclosed herein can further include containers comprising a solvent, such as, e.g., water or a buffered solution, e.g., saline. A solvent disclosed herein is useful to reconstitute a dried form of a composition disclosed herein.

The contents of the kit, including the container including one or more compositions or components disclosed herein, the delivery or application system, and instructions, are enclosed in an outer casing. The outer casing can be a box, a sealed bag, a foil pouch, etc. In certain embodiments, the delivery system, container, and instructions are enclosed in a box. In other embodiments of the kit, the container and instructions are contained in a first box, the delivery system is contained in a second box, and the first and second box are contained together in a third box

Aspects of the present specification disclose a method to clean, disinfect, sanitize, and/or sterilize a device. In one embodiment, a method disclosed herein comprises applying a composition disclosed herein to a device for a specified amount of time, wherein application results in the cleaning, disinfecting, sanitizing, and/or sterilizing of the device. In another aspect of this embodiment, a method disclosed herein further comprises rinsing the cleaned, disinfected, sanitized, and/or sterilized device with a rinse solution disclosed herein. Other aspects of the present specification disclose a composition disclosed herein for use to clean, disinfect, sanitize, and/or sterilize a device, including a medical device. Yet other aspects of the present specification disclose a use of a disclosed composition to clean, disinfect, sanitize, and/or sterilize a device, including a medical device.

Aspects of the present specification disclose a method to clean, disinfect, sanitize, and/or sterilize an endoscope. In an aspect of this embodiment, a method disclosed herein comprises applying a composition disclosed herein to an endoscope for a specified amount of time, wherein application results in the cleaning, disinfecting, sanitizing, and/or sterilizing of the endoscope. In another aspect of this embodiment, a method disclosed herein further comprises rinsing the cleaned, disinfected, sanitized, and/or sterilized endoscope with a rinse solution disclosed herein. Other aspects of the present specification disclose a composition disclosed herein for use to clean, disinfect, sanitize, and/or sterilize an endoscope. Yet other aspects of the present specification disclose a use of a disclosed composition to clean, disinfect, sanitize, and/or sterilize an endoscope.

A device, including a medical device, is cleaned by removal of visible soil, such as, e.g., organic and inorganic material, from objects and surfaces and normally is accomplished manually or mechanically. Thorough cleaning is essential before disinfection, sanitization, and sterilization because inorganic and organic materials that remain on the surfaces of a medical device interfere with the effectiveness of these processes.

A device, including a medical device is disinfected by eliminating many or all pathogenic microorganisms, except bacterial spores. Disinfection is less lethal than sterilization because it destroys most recognized pathogenic microorganisms but not necessarily all microbial forms (e.g., bacterial spores). A medical device is sterilized by destroying or eliminating all forms of microorganisms. Decontamination of a medical device removes pathogenic microorganisms from a medical device so that it is safe to handle, use, or discard.

A medical device is an instrument, apparatus, material, or other article, whether used alone or in combination, including software necessary for its application, intended by the manufacturer to be used for human beings for diagnosis, prevention, monitoring treatment, or alleviation of disease; diagnosis, monitoring, treatment, or alleviation of or compensation for an injury or handicap; investigation, replacement, or modification of the anatomy or of a physiologic process; or control of conception, and that does not achieve its primary intended action in or on the human body by pharmacologic, immunologic, or metabolic means but might be assisted in its function by such means. A medical device includes, without limitation, a surgical instrument, a respiratory therapy instrument, an anesthesia instrument, a catheter, an implant, a probe, an endoscope, an arthroscope, a laparoscope, a blade, a cystoscope, a spirometer, a CPAP mask and tubing, dialysis instrument and accessories, a heart-lung machine and accessories, a heart-lung bypass machine and accessories, and a diaphragm fitting ring. Non-limiting examples of a probe includes an ultrasound probe and an esophageal manometry probe. Non-limiting examples of a catheter includes a cardiac catheter, a urinary catheter, an anorectal manometry catheter. Non-limiting examples of an endoscope includes a gastrointestinal endoscope, a bronchoscope, and a nasopharyngoscope. Non-limiting examples of a blade includes a laryngoscope blade.

Aspects of the present specification disclose a method to clean, disinfect, sanitize, and/or sterilize a hard surface area. In an aspect of this embodiment, a method disclosed herein comprises applying a composition disclosed herein to a hard surface area for a specified amount of time, wherein application results in the cleaning, disinfecting, sanitizing, and/or sterilizing of the hard surface area. In another aspect of this embodiment, a method disclosed herein further comprises rinsing the cleaned, disinfected, sanitized, and/or sterilized surface with a rinse solution disclosed herein. Other aspects of the present specification disclose a composition disclosed herein for use to clean, disinfect, sanitize, and/or sterilize a hard surface area. Yet other aspects of the present specification disclose a use of a disclosed composition to clean, disinfect, sanitize, and/or sterilize a hard surface area. A hard surface area can be a porous surface area or a non-porous surface area.

A hard surface area can include any items present in a residence or a commercial, industrial and/or agricultural facility, such as, e.g., a hospital, a laboratory, a restaurant, an educational center, a food-processing facility, a dairy-processing facility, an airport, an oil field system, a sport facility, a shipping dock, a freight transport center, or any other commercial or industrial setting. A surface area can include any type of transportation carrier, such as, e.g., a water vessel like a boat, barge or ship, an aircraft like an airplane or helicopter, a ground vehicle like a motorcycle, car, truck or train, A surface area may be made of any material including brass, copper, aluminum, stainless steel, carbon steel, rubber, plastic, glass, wood, painted surface, or any combination thereof. A surface area includes, without limitation, a tabletop, countertop, floor, wall, ceiling, window, bed, gurney, door, door handle, shower, bath, sink, faucet, toilet, toilet seat, drain, equipment, machinery, personal protective gear, personal biohazard gear, and the like. A surface area may comprise a medical, dental, pharmaceutical, veterinary or mortuary device. A surface area may comprise human skin.

A composition disclosed herein can be applied according to a method or use disclosed herein to a hard surface area. Application of a composition disclosed herein can be by rubbing, pouring, sprinkling, misting, or spraying on, or otherwise applied to a hard surface area. A composition disclosed herein can be applied by introducing the composition into or onto a solid support such as, e.g., a wipe, a towelette, a towel, a mitt, a glove, or a mask and then applying the composition to a hard surface area. A composition disclosed herein can be applied by using a delivery device, such as, e.g., an aerosol dispenser, a pump spray, a trigger spray, or a squeeze bottle to apply the composition to a hard surface area.

A composition or component disclosed herein can be applied to a hard surface area according to a method or use disclosed herein as often as needed and/or desired. A composition disclosed herein can be applied to a hard surface area daily, every other day, every third of day, once a week, multiple times per week, once a month, multiple times per month, once a year or multiple times per year, as desired. A composition disclosed herein can be applied to a hard surface area multiple times per day, e.g., twice a day, three times a day, four time a day, five times a day, six times a day or as often as desired.

The presently disclosed compositions are useful in any application involving treating an individual. The presently disclosed compositions are useful in any application involving medical use, veterinarian use, or both. In one embodiment, a composition disclosed herein is useful in any application where hypochlorous acid is applied or administered.

In one embodiment, the presently disclosed compositions are useful in any application involving treating a tissue in an individual. For example, the compositions disclosed herein can be used to treat a wound by enhancing angiogenesis in an area in and around a wound or promoting rapid healing of a wound.

In one embodiment, the presently disclosed compositions are useful in any application involving treating a wound in an individual. For example, the compositions disclosed herein can be used to treat a wound by enhancing angiogenesis in an area in and around a wound or promoting rapid healing of a wound. A wound can be an open wound, a closed wound, or a burn. Non-limiting examples of an open wound include a laceration, an abrasion, an incision, a puncture, an avulsion, an ulcer, and a tear. Non-limiting examples of a closed wound includes a bruise, a contusion, and a hematoma.

Aspects of the present specification disclose a method of cleaning, disinfecting, sanitizing, and/or sterilizing a wound in an individual. In an aspect of this embodiment, a method disclosed herein comprises applying a composition disclosed herein to a wound for a specified amount of time, wherein application results in the cleaning, disinfecting, sanitizing, and/or sterilizing of the wound. In another aspect of this embodiment, a method disclosed herein further comprises rinsing the cleaned, disinfected, sanitized, and/or sterilized wound with a rinse solution disclosed herein. Other aspects of the present specification disclose a composition disclosed herein for use to clean, disinfect, sanitize, and/or sterilize a wound. Yet other aspects of the present specification disclose a use of a disclosed composition to clean, disinfect, sanitize, and/or sterilize a wound.

Aspects of the present specification disclose a method of treating a wound in an individual. In an aspect of this embodiment, a method disclosed herein comprises applying a composition disclosed herein to a wound for a specified amount of time, wherein application promotes healing of the wound. In another aspect of this embodiment, a method disclosed herein further comprises rinsing the treated wound with a rinse solution disclosed herein. Other aspects of the present specification disclose a composition disclosed herein for use to treat a wound. Yet other aspects of the present specification disclose a use of a disclosed composition to treat a wound. A wound can be an external wound on, e.g., a surface area of an individual or an internal wound located in the body or body cavity of an individual. A body cavity can be a space, compartment, or lumen, or potential space, compartment, or lumen in the body. A body cavity may include any space, compartment or lumen that contains organs, limbs, tissues, and surfaces within the body, or any space, compartment or lumen within organs, limbs, or tissues. A body cavity may include areas with the organs, for example an airway within the lungs, a nasal cavity, an oral cavity, a lumen of the gastrointestinal or excretory system including urethra and ureter, and spaces within the reproductive organs. A body cavity may be exposed to the outside of the body or may not be exposed to the outside space of the body. A body cavity any space created in the body. A body cavity may also include a space created in the body from injury or surgery or other artificial means.

In one embodiment, the presently disclosed compositions are useful in any application involving a microbial infection in an individual. A microbial infection includes a viral infection, a bacterial infection, and a fungal infection. A microbial infection can be an external infection on, e.g., a surface area of an individual or an internal infection, e.g., a mercer infection or sepsis, located in the body or body cavity of an individual.

Aspects of the present specification disclose a method of cleaning, disinfecting, sanitizing, sterilizing, and/or prevent a microbial infection in an individual. In an aspect of this embodiment, a method disclosed herein comprises applying a composition disclosed herein to a microbial infection for a specified amount of time, wherein application results in a cleaning, disinfecting, sanitizing, and/or sterilizing of the microbial infection. In another aspect of this embodiment, a method disclosed herein further comprises rinsing the cleaned, disinfected, sanitized, and/or sterilized microbial infection with a rinse solution disclosed herein. Other aspects of the present specification disclose a composition disclosed herein for use to clean, disinfect, sanitize, and/or sterilize a microbial infection. Yet other aspects of the present specification disclose a use of a disclosed composition to clean, disinfect, sanitize, and/or sterilize a microbial infection.

In one embodiment, a composition disclosed herein is used to clean, disinfect, sanitize, and/or treat a body part of an individual. Non-limiting examples of uses for a composition disclosed herein include cleaning/disinfecting tissue in wound care, pre-operative preparation, and surgery or other invasive procedure, cleaning/disinfecting a skin region in dermatological applications, cleaning/disinfecting the nasal or oral cavity, cleaning/disinfecting the lungs and respiratory tissues in respiratory applications, and cleaning/disinfecting the eye in ophthalmological applications.

Aspects of the present specification disclose a method of treating a microbial infection in an individual. In an aspect of this embodiment, a method disclosed herein comprises applying a composition disclosed herein to a microbial infection for a specified amount of time, wherein application results in a reduction, elimination and/or killing microbes causing the microbial infection. In another aspect of this embodiment, a method disclosed herein further comprises rinsing the treated microbial infection with a rinse solution disclosed herein. Other aspects of the present specification disclose a composition disclosed herein for use to treat a microbial infection. Yet other aspects of the present specification disclose a use of a disclosed composition to treat a microbial infection.

A dermatological application refers to cleaning/disinfecting a skin region of an individual of a microbial infection, such as, e.g., a viral, bacterial, or fungal infection. Non-limiting examples of a microbial infection of a skin region include urinary tract infection, eye lid wash, cataract treatment, warts, cutaneous leishmaniasis, candidal vulvovagititis, derrmatophytoses, bromhidrosis, pityriasis versicolor, acne vulgarish, rosasea, hydradenitis suppurativa, psoriasis, eczema, alopecia areata, oral lichen planus, xeroderma pigmentosum, actinic keratoses, melasma, keloids, and anti-aging.

A respiratory application refers to cleaning, disinfecting, treating the lungs, oral, nasal, and airways of an individual of a microbial infection, such as, e.g., a viral, bacterial, or fungal infection. A Respiratory application also refers to cleaning, disinfecting, treating the lungs, oral, nasal, and airways of an individual to prevent a microbial infection, such as, e.g., a viral, bacterial, or fungal infection. Non-limiting examples of a microbial infection include a respiratory infection, an oral infection, a nasal infection, and a sinus infection.

An ophthalmological application refers to cleaning, disinfecting, treating an eye of an individual of a microbial infection, such as, e.g., a viral, bacterial, or fungal infection. Non-limiting examples of a microbial infection of an eye include a bacterial conjunctivitis, a viral conjunctivitis, an epidemic keratoconjunctivitis, a pharyngoconjunctival fever, a stye, a blephartis, an episcleritis, a keratitis, a trachoma, and a corneal ulcer.

The presently disclosed compositions are useful in any application involving treating and/or providing relief of an inflammation, an ache, and/or a pain in an individual.

Aspects of the present specification disclose a method of treating an inflammation, an ache, and/or a pain in an individual. In an aspect of this embodiment, a method disclosed herein comprises applying a composition disclosed herein to an area of inflammation, an ache and/or a pain for a specified amount of time, wherein application results in a reduction and/or elimination of the pain. In another aspect of this embodiment, a method disclosed herein further comprises rinsing the treated area with a rinse solution disclosed herein. Other aspects of the present specification disclose a composition disclosed herein for use to treat an inflammation, an ache and/or a pain. Yet other aspects of the present specification disclose a use of a disclosed composition to treat an inflammation, an ache, and/or a pain.

The presently disclosed compositions are useful in enteral and parenteral applications, including oral, injectable, and topical applications. For example, the compositions disclosed herein can be used to clean, disinfect, sanitize, or sterilize a body region injured by a wound or infected with a microbe.

In an aspect of this embodiment, a method or use disclosed herein comprises applying a composition disclosed herein to an individual for a specified amount of time, wherein application results in the cleaning, disinfecting, sanitizing and/or sterilizing of a microbial infection in the individual. Other aspects of the present specification disclose a composition disclosed herein for use to clean, disinfect, sanitize, and/or sterilize a microbial infection in an individual. Yet other aspects of the present specification disclose a use of a disclosed composition to clean, disinfect, sanitize, and/or sterilize a microbial infection in an individual. Still other aspects of the present specification disclose a composition disclosed herein for the manufacture of a medicament to clean, disinfect, sanitize, and/or sterilize a microbial infection in an individual.

A method or use disclosed herein applies a composition disclosed herein to an individual. An individual refers to any animal including, without limitation, a fish, an amphibian, a bird, and a mammal. A mammal includes a human, a horse, a cow, a sheep, a dog, and a cat. As such, a method or use disclosed herein is for human use as well as veterinarian use.

In applications to an individual, a composition disclosed herein can be applied to a skin surface or can be internally administered. In one embodiment, a composition disclosed herein is applied topically to a skin region of an individual in order to clean, disinfect, sanitize, and/or sterilize the skin region. A skin region includes, without limitation, the face, forehead, lips, scalp, neck, shoulder, arms, hands, thighs, legs, knees, feet, chest, breast, back, groin, buttocks, and the like.

In one embodiment, a composition disclosed herein is internally administered to an individual. Such routes of administration include enteral routes of administration and parenteral routes of administration.

A composition disclosed herein can be applied according to a method or use disclosed herein to a skin region or body cavity, or body tissue. Application of a composition disclosed herein can be by rubbing, pouring, sprinkling, misting, nebulizing, or spraying on, or otherwise applied to the human body. A composition disclosed herein can be applied by introducing the composition into or onto a solid support such as, e.g., a wipe, a towelette, a towel, a mitt, a glove, or a mask and then applying the composition to a skin region. A composition disclosed herein can be applied by using a delivery device, such as, e.g., an aerosol dispenser, a misting dispenser, a nebulizing dispenser, a pump spray, a trigger spray, a squeeze bottle, a topical patch, a transdermal patch, or a dermal implant to apply the composition to a skin region.

A composition disclosed herein can be applied to an individual according to a method or use disclosed herein as often as needed and/or desired. A composition disclosed herein can be applied to an individual multiple times per day. A composition disclosed herein can be applied to an individual daily, every other day, every third of day, once a week, multiple times per week, once a month, multiple times per month, once a year or multiple times per year, as desired. A composition disclosed herein can be applied to an individual multiple times per day, e.g., twice a day, three times a day, four time a day, five times a day, six times a day or as often as desired.

The presently disclosed compositions are useful in food products, poultry, meat, fruit, vegetable production. For example, the compositions disclosed herein can be used to clean, disinfect, sanitize, or sterilize machinery, instruments, tables, rooms, including floors, ceilings and walls and any other hard surface from microbial contamination.

The presently disclosed compositions are useful in formulations on plants as a preservative or pesticide. For example, the compositions disclosed herein can be used to clean, disinfect, sanitize, or sterilize a plant injured by a wound or infected with a microbe.

The presently disclosed compositions are useful in formulations on cut flowers to prolong freshness and health. For example, the compositions disclosed herein can be used to clean, disinfect, sanitize, or sterilize a cut flower at a wound or to prevent microbial infection.

A disclosed method or use applies a composition disclosed herein for specified amount of time. In one embodiment, a specified amount of time is a time sufficient to clean a medical device, a surface, or an individual. In another embodiment, a specified amount of time is a time sufficient to disinfect a medical device, a surface, or an individual. In yet another embodiment, a specified amount of time is a time sufficient to sterilize a medical device, a surface, or an individual.

In aspects of this embodiment, a composition disclosed herein is applied to a device, like a medical device, a surface, or an individual for, e.g., about 15 seconds, about 30 seconds, about 45 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes. In other aspects of this embodiment, a composition disclosed herein is applied to a device, like a medical device, a surface, or an individual for, e.g., at least 15 seconds, at lease 30 seconds, at least 45 seconds, at least 1 minute, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, or at least 120 minutes. In yet other aspects of this embodiment, a composition disclosed herein is applied to device, like a medical device, a surface, or an individual for, e.g., at most 15 seconds, at most 30 seconds, at most 45 seconds, at most 1 minute, at most 5 minutes, at most 10 minutes, at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 70 minutes, at most 80 minutes, at most 90 minutes, at most 100 minutes, at most 110 minutes, or at most 120 minutes.

In still other aspects of this embodiment, a composition disclosed herein is applied to a device, like a medical device, a surface, or an individual for, e.g., about 15 seconds to 30 seconds, about 15 seconds to 45 seconds, about 15 seconds to 60 seconds, about 30 seconds to 60 seconds, about 45 seconds to 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 5 minutes to about 55 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 10 minutes to about 55 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 15 minutes to about 55 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 20 minutes to about 25 minutes, about 20 minutes to about 30 minutes, about 20 minutes to about 35 minutes, about 20 minutes to about 40 minutes, about 20 minutes to about 45 minutes, about 20 minutes to about 50 minutes, about 20 minutes to about 55 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 25 minutes to about 30 minutes, about 25 minutes to about 35 minutes, about 25 minutes to about 40 minutes, about 25 minutes to about 45 minutes, about 25 minutes to about 50 minutes, about 25 minutes to about 55 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 40 minutes to about 45 minutes, about 40 minutes to about 50 minutes, about 40 minutes to about 55 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 45 minutes to about 50 minutes, about 45 minutes to about 55 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, about 50 minutes to about 55 minutes, about 50 minutes to about 60 minutes, about 50 minutes to about 70 minutes, about 50 minutes to about 80 minutes, about 50 minutes to about 90 minutes, about 50 minutes to about 100 minutes, about 50 minutes to about 110 minutes, about 50 minutes to about 120 minutes, about 55 minutes to about 60 minutes, about 55 minutes to about 70 minutes, about 55 minutes to about 80 minutes, about 55 minutes to about 90 minutes, about 55 minutes to about 100 minutes, about 55 minutes to about 110 minutes, about 55 minutes to about 120 minutes, about 60 minutes to about 70 minutes, about 60 minutes to about 80 minutes, about 60 minutes to about 90 minutes, about 60 minutes to about 100 minutes, about 60 minutes to about 110 minutes, about 60 minutes to about 120 minutes, about 70 minutes to about 80 minutes, about 70 minutes to about 90 minutes, about 70 minutes to about 100 minutes, about 70 minutes to about 110 minutes, about 70 minutes to about 120 minutes, about 80 minutes to about 90 minutes, about 80 minutes to about 100 minutes, about 80 minutes to about 110 minutes, about 80 minutes to about 120 minutes, about 90 minutes to about 100 minutes, about 90 minutes to about 110 minutes, about 90 minutes to about 120 minutes, about 100 minutes to about 110 minutes, about 100 minutes to about 120 minutes, or about 110 minutes to about 120 minutes.

A disclosed composition, method or use disclosed herein are less harsh on a medical device resulting in a longer lifetime use of a medical device. In aspects of this embodiment, a medical device can be cleaned, disinfected, sanitized, and/or sterilized, e.g., about 50 times, about 60 times, about 70 times, about 80 times, about 90 times, about 100 times, about 110 times, about 120 times, about 130 times, about 140 times, about 150 times, about 160 times, about 170 times, about 180 times, about 190 times, about 200 times, about 210 times, about 220 times, about 230 times, about 240 times, about 250 times, about 260 times, about 270 times, about 280 times, about 290 times, or about 300 times. In other aspects of this embodiment, a medical device can be cleaned, disinfected, sanitized, and/or sterilized, e.g., at least 50 times, at least 60 times, at least 70 times, at least 80 times, at least 90 times, at least 100 times, at least 110 times, at least 120 times, at least 130 times, at least 140 times, at least 150 times, at least 160 times, at least 170 times, at least 180 times, at least 190 times, at least 200 times, at least 210 times, at least 220 times, at least 230 times, at least 240 times, at least 250 times, at least 260 times, at least 270 times, at least 280 times, at least 290 times, or at least 300 times. In yet other aspects of this embodiment, a medical device can be cleaned, disinfected, sanitized, and/or sterilized, e.g., at most 50 times, at most 60 times, at most 70 times, at most 80 times, at most 90 times, at most 100 times, at most 110 times, at most 120 times, at most 130 times, at most 140 times, at most 150 times, at most 160 times, at most 170 times, at most 180 times, at most 190 times, at most 200 times, at most 210 times, at most 220 times, at most 230 times, at most 240 times, at most 250 times, at most 260 times, at most 270 times, at most 280 times, at most 290 times, or at most 300 times.

In still other aspects of this embodiment, a medical device can be cleaned, disinfected, sanitized, and/or sterilized, e.g., about 50 times to about 60 times, about 50 times to about 70 times, about 50 times to about 80 times, about 50 times to about 90 times, about 50 times to about 100 times, about 50 times to about 110 times, about 50 times to about 120 times, about 50 times to about 130 times, about 50 times to about 140 times, about 50 times to about 150 times, about 50 times to about 175 times, about 50 times to about 200 times, about 50 times to about 225 times, about 50 times to about 250 times, about 50 times to about 275 times, about 50 times to about 300 times, about 75 times to about 90 times, about 75 times to about 100 times, about 75 times to about 110 times, about 75 times to about 120 times, about 75 times to about 130 times, about 75 times to about 140 times, about 75 times to about 150 times, about 75 times to about 175 times, about 75 times to about 200 times, about 75 times to about 225 times, about 75 times to about 250 times, about 75 times to about 275 times, about 75 times to about 300 times, about 100 times to about 110 times, about 100 times to about 120 times, about 100 times to about 130 times, about 100 times to about 140 times, about 100 times to about 150 times, about 100 times to about 175 times, about 100 times to about 200 times, about 100 times to about 225 times, about 100 times to about 250 times, about 100 times to about 275 times, about 100 times to about 300 times, about 125 times to about 150 times, about 125 times to about 175 times, about 125 times to about 200 times, about 125 times to about 225 times, about 125 times to about 250 times, about 125 times to about 275 times, about 125 times to about 300 times, about 150 times to about 175 times, about 150 times to about 200 times, about 150 times to about 225 times, about 150 times to about 250 times, about 150 times to about 275 times, about 150 times to about 300 times, about 175 times to about 200 times, about 175 times to about 225 times, about 175 times to about 250 times, about 175 times to about 275 times, about 175 times to about 300 times, about 200 times to about 225 times, about 200 times to about 250 times, about 200 times to about 275 times, about 200 times to about 300 times, about 225 times to about 250 times, about 225 times to about 275 times, about 225 times to about 300 times, about 250 times to about 275 times, about 250 times to about 300 times, or about 275 times to about 300 times.

A method or use disclosed herein may further comprise a rinsing step using a rinse solution. Typically, the rinse solution is used to rinse a cleaned, disinfected, sanitized, and/or sterilized medical device or surface. The rinse solution is preferably a sterile solution. In one embodiment, a rinse solution disclosed herein comprises water. In another embodiment, a rinse solution disclosed herein comprises hypochlorous acid or free available chlorine and water. In another embodiment, a rinse solution disclosed herein does not comprises hypochlorous acid and/or free available chlorine. When present in a kit disclosed herein, the rinse solution is present in a separate container.

The amount of hypochlorous acid or free available chlorine present in a rinse solution disclosed herein is any amount that provides an antimicrobial effect, with the proviso that the total amount of hypochlorous acid or free available chlorine present is an amount below the threshold level that results in oxidation of a medical device of surface as disclosed herein. In aspects of this embodiment, the amount of hypochlorous acid or free available chlorine present in a rinse solution may be, e.g., about 5 ppm, about 10 ppm, about 20 ppm, about 30 ppm, about 40 ppm, about 50 ppm, about 60 ppm, about 70 ppm, about 80 ppm, about 90 ppm, about 100 ppm, about 110 ppm, or about 120 ppm. In other aspects of this embodiment, the amount of hypochlorous acid or free available chlorine present in a rinse solution may be, e.g., at most 5 ppm, at most 10 ppm, at most 20 ppm, at most 30 ppm, at most 40 ppm, at most 50 ppm, at most 60 ppm, at most 70 ppm, at most 80 ppm, at most 90 ppm, at most 100 ppm, at most 110 ppm, or at most 120 ppm. In yet other aspects of this embodiment, the amount of hypochlorous acid or free available chlorine present in a rinse solution may be, e.g., about 5 ppm to about 10 ppm, about 5 ppm to about 20 ppm, about 5 ppm to about 30 ppm, about 5 ppm to about 40 ppm, about 5 ppm to about 50 ppm, about 5 ppm to about 60 ppm, about 5 ppm to about 70 ppm, about 5 ppm to about 80 ppm, about 5 ppm to about 90 ppm, about 5 ppm to about 100 ppm, about 5 ppm to about 110 ppm, about 5 ppm to about 120 ppm, about 10 ppm to about 20 ppm, about 10 ppm to about 30 ppm, about 10 ppm to about 40 ppm, about 10 ppm to about 50 ppm, about 10 ppm to about 60 ppm, about 10 ppm to about 70 ppm, about 10 ppm to about 80 ppm, about 10 ppm to about 90 ppm, about 10 ppm to about 100 ppm, about 10 ppm to about 110 ppm, about 10 ppm to about 120 ppm, about 20 ppm to about 30 ppm, about 20 ppm to about 40 ppm, about 20 ppm to about 50 ppm, about 20 ppm to about 60 ppm, about 20 ppm to about 70 ppm, about 20 ppm to about 80 ppm, about 20 ppm to about 90 ppm, about 20 ppm to about 100 ppm, about 20 ppm to about 110 ppm, about 20 ppm to about 120 ppm, about 30 ppm to about 40 ppm, about 30 ppm to about 50 ppm, about 30 ppm to about 60 ppm, about 30 ppm to about 70 ppm, about 30 ppm to about 80 ppm, about 30 ppm to about 90 ppm, about 30 ppm to about 100 ppm, about 30 ppm to about 110 ppm, about 30 ppm to about 120 ppm, about 40 ppm to about 50 ppm, about 40 ppm to about 60 ppm, about 40 ppm to about 70 ppm, about 40 ppm to about 80 ppm, about 40 ppm to about 90 ppm, about 40 ppm to about 100 ppm, about 40 ppm to about 110 ppm, about 40 ppm to about 120 ppm, about 50 ppm to about 60 ppm, about 50 ppm to about 70 ppm, about 50 ppm to about 80 ppm, about 50 ppm to about 90 ppm, about 50 ppm to about 100 ppm, about 50 ppm to about 110 ppm, about 50 ppm to about 120 ppm, about 60 ppm to about 70 ppm, about 60 ppm to about 80 ppm, about 60 ppm to about 90 ppm, about 60 ppm to about 100 ppm, about 60 ppm to about 110 ppm, about 60 ppm to about 120 ppm, about 70 ppm to about 80 ppm, about 70 ppm to about 90 ppm, about 70 ppm to about 100 ppm, about 70 ppm to about 110 ppm, about 70 ppm to about 120 ppm, about 80 ppm to about 90 ppm, about 80 ppm to about 100 ppm, about 80 ppm to about 110 ppm, about 80 ppm to about 120 ppm, about 90 ppm to about 100 ppm, about 90 ppm to about 110 ppm, about 90 ppm to about 120 ppm, about 100 ppm to about 110 ppm, about 100 ppm to about 120 ppm, or about 110 ppm to about 120 ppm.

In some embodiments a composition disclosed herein comprises about 30 ppm to about 1,000 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 1,000 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 700 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.5% to about 1.0% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.6% to about 0.9% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 425 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments a composition disclosed herein comprises about 30 ppm to about 400 ppm of stable free available chlorine, one or more peroxides, and about 0.7% to about 0.8% sodium chloride and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.5% to about 1.0% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.6% to about 0.9% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 425 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments a composition disclosed herein comprises about 30 ppm to about 400 ppm of stable free available chlorine, one or more peroxides, and about 0.7% to about 0.8% sodium chloride and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 500 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 475 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 30 ppm to about 450 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 10 ppm to 300 ppm of copper chloride, 10 ppm to 300 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 25 ppm to 275 ppm of copper chloride, 25 ppm to 275 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 30 ppm to 250 ppm of copper chloride, 30 ppm to 250 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 40 ppm to 200 ppm of copper chloride, 40 ppm to 200 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 50 ppm to 150 ppm of copper chloride, 50 ppm to 150 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 60 ppm to 125 ppm of copper chloride, 60 ppm to 125 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 60 ppm to 100 ppm of copper chloride, 60 ppm to 100 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 275 mOsm/kg to about 295 mOsm/kg or about 300 mOsm/L to about 316 mOsm/L. In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 275 mOsm/kg to about 295 mOsm/kg or about 300 mOsm/L to about 316 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.5% to about 1.0% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.6% to about 0.9% sodium chloride, and has an osmolality of about 280 mOsm/kg to about 290 mOsm/kg or about 300 mOsm/L to about 310 mOsm/L. In some embodiments a composition disclosed herein comprises about 100 ppm to about 425 ppm of stable free available chlorine, one or more peroxides, 70 ppm to 90 ppm of copper chloride, 70 ppm to 90 ppm of zinc chloride, and about 0.7% to about 0.8% sodium chloride, and has an osmolality of about 280 mOsm/kg to about 290 mOsm/kg or about 300 mOsm/L to about 310 mOsm/L. In some embodiments, the concentration of one or more peroxides is the same as the concentration of the one or more free available chlorine species present in a composition disclosed herein. In some embodiments, the concentration of one or more peroxides present is less than the concentration of the one or more free available chlorine species present in a composition disclosed herein. In some embodiments, the concentration of one or more peroxides present is more than the concentration of the one or more free available chlorine species present in a composition disclosed herein.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions, or methods and uses disclosed herein.

### Example 1

### Preparation of Stable Free Available Chlorine Solution

This example illustrates how to prepare a composition disclosed herein of a solution stably comprising together one or more stable free available chlorine species and one or more peroxides.

A solution comprising one or more stable free available chlorine species and one or more peroxides was produced by hydrolysis procedure 1. Initially, an aqueous solution is prepared by adding about 0.04% (w/w) to about 0.2% (w/w) sodium peroxide to purified water. Optionally, sodium phosphate and/or sodium chloride is added in an amount sufficient to achieve up to 200 ppm to 600 ppm of each salt in the produced composition. The pH of this solution is then adjusted to a pH range of about 5.0 to about 5.8, preferably to a pH of about 5.2 to about 5.6, using sodium hydroxide or any other suitable base. Chlorine gas (Cl₂) was then introduced to the solution at a rate of 240 g/hour to 320 g/hour for about 30 minutes to 60 minutes to produce a first reaction solution. Analysis of this first reaction solution revealed that it comprised about 10 ppm to about 40 ppm of one or more free available chlorine species. Once this reaction step was completed, an additional 0.001% to 0.03% sodium peroxide was added to the first reaction solution. Analysis indicated that this peroxide addition had the effect of increasing the pH to above 9 and eliminating the detection of the one or more free available chlorine species previously produced. Chlorine gas (Cl₂) was then introduced to the first reaction solution at a rate of 240 g/hour to 320 g/hour for about 10 minutes to about 20 minutes to produce a second reaction solution having the desired concentration of the one or more free available chlorine species. Analysis of this second reaction solution revealed that it comprised about 120 ppm to about 240 ppm of one or more free available chlorine species. In addition, given the starting amount of sodium peroxide, empirically the concentration of one or more peroxides present is in the second reaction solution is at most equivalent to the concentration of the one or more free available chlorine species present in the second reaction solution, although some loss of the one or more peroxides does occurs by degassing, and as such the concentration of one or more peroxides present is in the second reaction solution is less than the concentration of the one or more free available chlorine species present in the second reaction solution. The resulting reactions produced a solution comprising stably containing free available chlorine species and one or more peroxides.

In further experiments, the peroxide addition and chlorine gassing process were conducted a third time. In this step, once this second reaction step was completed, an additional 0.001% to 0.02% sodium peroxide was added to the second reaction solution. Analysis indicated that this peroxide addition had the effect of increasing the pH to above 9 and eliminating the detection of the one or more free available chlorine species previously produced. Chlorine gas (Cl₂) was then introduced to the second reaction solution at a rate of 240 g/hour to 320 g/hour for about 2 minutes to about 10 minutes to produce a third reaction solution having the desired concentration of the one or more free available chlorine species. Analysis of this third reaction solution revealed that it comprised about 160 ppm to about 300 ppm of one or more free available chlorine species. In addition, given the starting amount of sodium peroxide, empirically the concentration of one or more peroxides present is in the third reaction solution is at most equivalent to the concentration of the one or more free available chlorine species present in the third reaction solution, although some loss of the one or more peroxides does occurs by degassing, and as such the concentration of one or more peroxides present is in the third reaction solution is less than the concentration of the one or more free available chlorine species present in the third reaction solution. The resulting reaction produced a solution stably comprising together one or more stable free available chlorine species and one or more peroxides.

In an alternative method a solution comprising one or more stable free available chlorine species and one or more peroxides was produced by hydrolysis procedure 2. Initially, an aqueous solution is prepared by adding about 0.03% to 0.1% sodium chloride (NaCl) to purified water. Both chlorine (Cl₂) and oxygen (O₂) gases are then introduced to the solution at a rate of 240 g/hour to 320 g/hour for about 30 minutes to 60 minutes to produce a first reaction solution. Analysis of this first reaction solution reveals that it comprises about 10 ppm to about 40 ppm of one or more free available chlorine species and had a pH of about 3.3. Once this reaction step is completed, an additional 0.01% to 0.03% sodium chloride (NaCl) is added to the first reaction solution. Both chlorine (Cl₂) and oxygen (O₂) gases are then introduced to the first reaction solution at a rate of 240 g/hour to 320 g/hour for about 10 minutes to 20 minutes to produce a second reaction solution having the desired concentration of the one or more free available chlorine species. Analysis of this second reaction solution reveals that it comprises about 120 ppm to about 240 ppm of one or more free available chlorine species. In addition, given the starting amount of sodium peroxide, empirically the concentration of one or more peroxides present is in the second reaction solution is at most equivalent to the concentration of the one or more free available chlorine species present in the second reaction solution, although some loss of the one or more peroxides does occurs by degassing, and as such the concentration of one or more peroxides present is in the second reaction solution is less than the concentration of the one or more free available chlorine species present in the second reaction solution. The resulting reactions will produce a solution stably comprising together one or more stable free available chlorine species and one or more peroxides.

In another alternative method, a solution comprising one or more stable free available chlorine species and one or more peroxides was produced by hydrolysis procedure 3. Initially, an aqueous solution is prepared by adding about 0.03% to about 0.2% of one or more peroxides and about 0.5% to about 1.0% of sodium chloride to purified water. Optionally, sodium phosphate is added in an amount sufficient to achieve up to 200 ppm to 600 ppm of the salt in the produced composition. The pH of this solution in then adjusted to a pH range of about 5.0 to about 5.8, preferably to a pH of about 5.2 to about 5.6, using sodium hydroxide or any other suitable base. Chlorine (Cl₂) gas is then introduced to the solution at a rate of 240 g/hour to 320 g/hour for about 30 minutes to 60 minutes to produce a first reaction solution. Analysis of this first reaction solution reveals that it comprises about 10 ppm to about 40 ppm of one or more free available chlorine species and had a pH of about 3.3. Once this reaction step is completed, an additional 0.01% to 0.03% of one or more peroxides and sodium hydroxide is added to the first reaction solution. Chlorine (Cl₂) gas is then introduced to the first reaction solution at a rate of 240 g/hour to 320 g/hour for about 10 minutes to 20 minutes to produce a second reaction solution. Analysis of this second reaction solution reveals that it comprises about 120 ppm to about 240 ppm of one or more free available chlorine species having the desired concentration of the one or more free available chlorine species. In addition, given the starting amount of sodium peroxide, empirically the concentration of one or more peroxides present is in the second reaction solution is at most equivalent to the concentration of the one or more free available chlorine species present in the second reaction solution, although some loss of the one or more peroxides does occurs by degassing, and as such the concentration of one or more peroxides present is in the second reaction solution is less than the concentration of the one or more free available chlorine species present in the second reaction solution. The resulting reactions will produce a solution stably comprising together one or more stable free available chlorine species and one or more peroxides.

Optionally, a solution stably comprising together one or more stable free available chlorine species and one or more peroxides produced by any one of hydrolysis procedures 1-3 can further comprise a step of adding additional sodium chloride to adjust the osmolality of the solution. In this step, a solution stably comprising together one or more stable free available chlorine species and one or more peroxides was degassed for 24 hours to 72 hours. After degassing, sodium chloride was added in an amount of about 0.5% to about 1.0% to the resulting composition to adjust the osmolality to a range of about 275 mOsm/kg to about 295 mOsm/kg.

### Example 2

### Preparation of Stable Free Available Chlorine Solution

This example illustrates how to prepare a composition disclosed herein of a solution stably comprising together one or more stable free available chlorine species and one or more peroxides.

A solution comprising one or more stable free available chlorine species and one or more peroxides was produced by hydrolysis procedure 4. Initially, an aqueous solution is prepared by adding about 0.08% (w/w) sodium peroxide to purified water. Optionally, sodium phosphate and/or sodium chloride is added in an amount sufficient to achieve up to 200 ppm to 600 ppm of each salt in the produced composition. Chlorine gas (Cl₂) was then introduced to the solution at a rate of 280 g/hour for about 30 minutes to 60 minutes to produce a first reaction solution. Analysis of this first reaction solution revealed that it comprised about 10 ppm to about 40 ppm of one or more free available chlorine species and had a pH of about 8 to 10. Once this reaction step was completed, an additional 0.013% sodium peroxide was added to the first reaction solution. Analysis indicated that this peroxide addition had the effect of increasing the pH to above 11 and eliminated the detection of the one or more free available chlorine species previously produced. Chlorine gas (Cl₂) was then introduced to the solution at a rate of 280 g/hour for about 30 minutes to 60 minutes to produce a second reaction solution of the one or more free available chlorine species. Analysis of this second reaction solution revealed that it comprised about 500 ppm to about 700 ppm of one or more free available chlorine species. In addition, given the starting amount of sodium peroxide, empirically the concentration of one or more peroxides present is in the second reaction solution is at most equivalent to the concentration of the one or more free available chlorine species present in the second reaction solution, although some loss of the one or more peroxides does occurs by degassing, and as such the concentration of one or more peroxides present is in the second reaction solution is less than the concentration of the one or more free available chlorine species present in the second reaction solution. The resulting reactions produced a solution stably comprising together one or more stable free available chlorine species and one or more peroxides.

Optionally, a solution stably comprising together one or more stable free available chlorine species and one or more peroxides produced by any one of hydrolysis procedure 4 can further comprise a step of adding additional sodium chloride to adjust the osmolality of the solution. In this step, a solution stably comprising together one or more stable free available chlorine species and one or more peroxides was degassed for 48 hours. After degassing, sodium chloride was added in an amount of about 0.76% to the resulting composition to adjust the osmolality to a range of about 286 mOsm/kg.

### Example 3

### Bacterial Viability Assays

This example illustrates how to conduct a bacterial viability assay using a composition disclosed herein.

Fresh 24-hour culture of Staphylococcus aureus (ATCC 29213) was prepared on Tryptic Soy Agar (TSA) at 36±1° C. A bacterial inoculum was prepared using a colorimeter to confirm a yield of 1×10⁷ CFU/mL.

Compositions MC-A35 (stored for 6 months), MC-A35 (stored 1 week), and MCN-A100 (stored 1 week) were tested against S. aureus in presence of 2% bovine serum for a time-point of 5 minutes. Composition MC-A35 comprises 160 ppm stable free available chlorine and at most 160 ppm of one or more peroxides while MCN-A100 comprises 160 ppm stable free available chlorine, at most 160 ppm of one or more peroxide, and 0.75% sodium chloride and has an osmolality of 286 mOsm/kg.

Samples were tested as follows: Each assay contained 50 μL of pathogenic bacteria suspension, 100 μL of bovine serum and 4,850 μL of a composition formulation disclosed herein. Samples were exposed for 5 minutes prior to adding a neutralizing agent used to inhibit the activity of the microbial agent; plates were incubated at 30° C. to 35° C. for up to 2 days. The presence of viable bacteria was then measured by counting the number of colonies observed on the agar medium and bacterial log reduction in comparison to growth control was calculated and results are shown in Table 20.

TABLE 1
Bacterial Viability Assay

| | Log Growth Reduction^{a, b} |
|---|---|
| Formulation | 2% Serum |
| MC-A35 | 1.2 × 10⁴ |
| MC-A35 | 4.5 × 10⁴ |
| MCN-A100 | 9.5 × 10⁷ |

^{a}A minimum of 3 log reduction in bacterial growth represents ≥99.9% kill of bacteria.
^{b}Positive controls for all assays conducted exhibited 0 log reduction in bacterial growth.

These experiments demonstrate two significant characteristics. First, when comparing the MC-A35 composition stored for 6 months versus the newly prepared MC-A35 composition there was no significant loss in efficacy. For example, both compositions exhibited a 4-log reduction in bacterial growth. This result indicates that Composition MC-A35 shows significant stability over time. Second, the addition of sodium chloride to adjust the composition to a physiological osmolality (286 mOsm/kg) significantly improved the efficacy of the composition. For example, Composition MCN-A100 exhibited a 7-log reduction in bacterial growth, which is a three log increase relative to both MC-A35 compositions tested. The dramatic increase in efficacy by simply adjusting MC-A35 to a physiological osmolality to produce Composition MCN-A100) is a surprising and unexpected result.

### Example 4

### Bacterial Viability Assays

This example illustrates how to conduct a bacterial viability assay using a composition disclosed herein of a solution stably comprising together one or more stable free available chlorine species and one or more peroxides.

To prepare fresh cultures of pathogenic bacteria, 10 mL nutrient broth was inoculated using a stock culture of Staphylococcus epidermidis (ATCC BAA-1707) and incubated on an orbital shaker for 24±2 hours at approximately 150 rpm at 36±1° C. This culture was used to inoculate Tryptic Soy Agar (TSA) plates. Each plate was inoculated with the culture. Each plate was incubated for up to 5 days at 30° C. to 35° C. Following incubation, suspensions were prepared by adding growth from TSA plates into a tube containing phosphate buffer. Each tube was then vortexed, and an aliquot was serially diluted and plated onto TSA plate for further numeration. The final numeration count uses aliquot volume, count obtained on TSA plate and the total original volume of culture. The remaining amount in the tube was used to inoculate actual test samples.

The following compositions were tested against S. aureus in presence of 5% serum. Test Solution 1 comprised 125 ppm of one or more stable free available chlorine species and at most 125 ppm of one or more peroxides. Test Solution 2 comprised 125 ppm of one or more stable free available chlorine species, at most 125 ppm of one or more peroxides, and 0.76% sodium chloride having an osmolarity of 286 mOsm/kg.

Assay samples were prepared as follows: Each test solution contained 100 μL of a pathogenic bacteria suspension, 500 μL of serum, and 9,400 μL of either Test Solution 1 or Test Solution 2. A control solution was prepared by substituting the test solution for TSA media. Assay samples were incubated for 15 minutes at which point a neutralizing agent was used to inhibit the activity of the composition being tested. A 10-fold serial dilution of the neutralized solution was then prepared in six test tubes to produce a 6-log range of the assay sample. An aliquot of each dilution was plated on petri dishes containing TSA medium and the cultures incubated for 48±2 hours at 36±1° C. Bacterial titers were determined by counting the number of colonies observed on the medium and bacterial log reduction was calculated in comparison to growth controls. The results are shown in Table 2.

TABLE 2
Bacterial Viability Assay

| | Log Growth Reduction^{a, b} |
|---|---|
| Formulation | 5% Serum |
| Control Solution | 1 × 10⁰ |
| Test Solution 1 | 1 × 10⁴ |
| Test Solution 1 | 1 × 10⁷ |

^{a}A minimum of 3 log reduction in bacterial growth represents ≥99.9% kill of bacteria.
^{b}Positive controls for all assays conducted exhibited 0 log reduction in bacterial growth.

These experiments demonstrate that the addition of sodium chloride to adjust the composition to a physiological osmolality (286 mOsm/kg) significantly improved the efficacy of the composition. For example, Test Solution 2 exhibited a 7-log reduction in bacterial growth, which is a three log increase relative to Test Solution 1, which demonstrated a 4-log reduction in bacterial growth. The dramatic increase in efficacy by simply adjusting a composition disclosed herein to a physiological osmolality to produce Test Solution 2 is a surprising and unexpected result.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention and such examples are not limiting thereto. As such, the specific embodiments are not intended to be exhaustive or to limit the invention to the precise forms disclosed. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims. In addition, all methods described herein can be performed in any suitable order unless otherwise indicated herein or otherwise clearly contradicted by context. Therefore, it should be understood that embodiments of the disclosed subject matter are in no way limited to a particular element, compound, composition, component, article, apparatus, methodology, use, protocol, step, and/or limitation described herein, unless expressly stated as such.

While aspects of the invention have been described with reference to at least one exemplary embodiment, it is to be clearly understood by those skilled in the art that the invention is not limited thereto. Furthermore, those of ordinary skill in the art will recognize that certain changes, modifications, permutations, alterations, additions, subtractions, and sub-combinations thereof can be made in accordance with the teachings herein without departing from the spirit of the present invention. It is intended that the following appended claims and claims hereafter introduced are interpreted to include all such changes, modifications, permutations, alterations, additions, subtractions, and sub-combinations as are within their true spirit and scope. Accordingly, the scope of the present invention is not to be limited to that precisely as shown and described by this specification. Rather, the scope of the invention is to be interpreted only in conjunction with the appended claims and it is made clear, here, that the inventor(s) believe that the claimed subject matter is the invention.

Certain embodiments of the present invention are described herein, including the best mode known to the inventors for conducting the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step, or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more, or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, all lists of values should be considered as inclusive of intermediate values unless the context indicates the contrary.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a comparable manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the methods and the order in which the respective elements of each method are performed are purely exemplary. Depending on the implementation, they may be performed in any order or in parallel, unless indicated otherwise in the present disclosure.

Finally, all patents, patent publications, and other references cited and identified in the present specification are individually and expressly incorporated herein by reference in their entirety to the same extent as if each individual publication or patent application were specifically and individually indicated to be incorporated by reference. These publications are provided solely for their disclosure prior to the filing date of the present application. The reference to any prior art in this specification is not, and should not be taken as, an acknowledgement or any form of suggestion that the prior art forms part of the common general knowledge from any country. In addition, where a definition or use of a term in an incorporated reference is inconsistent or contrary to the definition of that term provided herein, the definition of that term provided herein applies and the definition of that term in the reference does not apply. Lastly, nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A composition comprising:
70 ppm to 1,000 ppm of one or more stable free available chlorine species, the one or more stable free available chlorine species including at least 70 ppm hypochlorous acid,
one or more peroxides, and
sodium chloride,
wherein the one or more peroxides and the one or more stable free available chlorine coexist, and
wherein the composition has an osmolality of about 275 mOsm/kg to about 295 mOsm/kg.

2. (canceled)

3. The composition according to claim 1, wherein the one or more stable free available chlorine species is present in an amount of 70 ppm to 700 ppm.

4. The composition according to claim 3, wherein the one or more stable free available chlorine species is present in an amount of 100 ppm to 500 ppm.

5-6. (canceled)

7. The composition according to claim 1, wherein the one or more stable free available chlorine species comprises hypochlorous acid, hypochlorite (OCl⁻), chlorine gas (Cl₂), superchloride (Cl₂⁻), chlorine oxide radical (ClO), peroxyhypochlorous acid (HOOCl), another reactive chlorine species (RCS), or a combination thereof.

8. The composition according to claim 1, wherein the one or more peroxides is present in an amount equivalent to the amount of the one or more stable free available chlorine species, or
wherein the one or more peroxides is present in an amount that is less than the amount as the one or more stable free available chlorine species, or
wherein the one or more peroxides is present in an amount that is more than the amount as the one or more stable free available chlorine species.

9-11. (canceled)

12. The composition according to claim 1, wherein the one or more peroxides is present in an amount of 30 ppm to 700 ppm.

13-14. (canceled)

15. The composition according to claim 12, wherein the one or more peroxides is present in an amount of 100 ppm to 425 ppm.

16. The composition according to claim 1, wherein the one or more peroxides comprise hydrogen peroxide, sodium peroxide, calcium peroxide, lithium peroxide, potassium peroxide, other alkali metal peroxides, other alkali earth metal peroxides, or a combination thereof.

17. The composition according to claim 1, wherein the sodium chloride is present in an amount of about 0.5% to about 1.0% by weight of the composition.

18. The composition according to claim 17, wherein the sodium chloride is present in an amount of about 0.7% to about 1.0% by weight of the composition.

19-20. (canceled)

21. The composition according to claim 1, wherein the osmolality is from about 280 mOsm/kg to about 290 mOsm/kg.

22. (canceled)

23. The composition according to claim 1, further comprising one or more reactive oxygen species.

24. The composition according to claim 23, wherein the reactive oxygen species comprise oxygen gas (O₂), superoxide (O₂⁻), singlet oxygen (¹O₂), α-oxygen (α-O), protonated oxygen (HO₂⁺), hydroperoxyl radical (HO₂), hydroxyl radical (OH), peroxide (O₂⁻²), ozone (O₃), ozonide (O₃⁻), or a combination thereof.

25-38. (canceled)

39. The composition according to claim 1, comprising about 100 ppm to about 425 ppm of stable free available chlorine species, one or more peroxides, and about 0.7% to about 0.9% by weight of sodium chloride, the composition having an osmolality of about 280 mOsm/kg to about 290 mOsm/kg.

40. The composition according to claim 1, comprising about 100 ppm to about 425 ppm of stable free available chlorine species, one or more peroxides, and about 0.7% to about 0.9% by weight of sodium chloride, the composition having an osmolality of about 285 mOsm/kg to about 287 mOsm/kg.

41. A method of manufacture a composition, the method comprising
adding sodium peroxide to purified water contained in a vessel to prepare an aqueous solution,
introducing chlorine gas to the aqueous solution under hydrolysis conditions for a first period of time to produce a first reaction solution comprising one or more stable free available chlorine species and one or more peroxides.

42-43. (canceled)

44. The method according to claim 41, further comprising adding sodium chloride to the first reaction solution to adjust the osmolality to a physiological compatible osmolality.

45-46. (canceled)

47. A method according to claim 41, further comprising
adding sodium peroxide to the first reaction solution, and
introducing chlorine gas to the first reaction solution under hydrolysis conditions for a second period of time to produce a second reaction solution comprising one or more stable free available chlorine species and one or more peroxides.

50-52. (canceled)

53. A method according to claim 47, further comprising
adding sodium peroxide to the second reaction solution, and
introducing chlorine gas to the second reaction solution under hydrolysis conditions for a third period of time to produce a third reaction solution comprising one or more stable free available chlorine species and one or more peroxides.

54-68. (canceled)

69. A method of treating, preventing, and/or providing relief to an inflammatory response in a respiratory system of an individual, the method comprising administering to the individual a composition according to claim 1.

70-72. (canceled)
